(19) United States
(12) Patent Application Publication
(10) Pub. No.: US 2024/0315992 A1
(43) Pub. Date: Sep. 26, 2024

(54) AMINOXY ACID-BASED ANTIBACTERIAL COMPOUNDS AND METHODS THEREOF

(71) Applicant: The University of Hong Kong, Hong Kong (CN)

(72) Inventors: Dan YANG, Hangzhou (CN); Pak Ming FONG, Hong Kong (CN); Richard Yi Tsun KAO, Hong Kong (CN)

(21) Appl. No.: 18/179,840

(22) Filed: Mar. 7, 2023

(51) Int. Cl. A61K 31/166; A61K 31/36; A61K 31/404; A61K 31/405; A61K 31/426; A61K 31/505; A61K 31/5375; A61K 31/7036; A61P 31/04; C07C 259/10; C07D 209/18; C07D 239/42; C07D 277/46; C07D 295/135; C07D 295/16; C07D 317/66

(52) U.S. Cl. CPC A61K 31/166; A61K 31/36; A61K 31/404; A61K 31/405; A61K 31/426; A61K 31/505; A61K 31/5375; A61K 31/7036; A61P 31/04; C07C 259/10; C07D 209/18; C07D 239/42; C07D 277/46; C07D 295/135; C07D 295/16; C07D 317/66

(57) ABSTRACT

The subject invention pertains to novel compounds, compositions containing the novel compounds, and methods for targeting gram-positive bacteria with the compounds or compositions of the subject invention. The subject invention further pertains to novel compounds, novel compositions, and methods for targeting gram-positive bacteria persister cells and gram-positive bacterial biofilms. The subject compounds and compositions can target gram-positive bacteria by modulating the proton motive force, calcium influx, autolysis, or any combination thereof. The novel compounds are synthetic, small molecules that can induce the death of gram-positive bacteria.

The Sequence Listing for this application is labeled “UHK.318.xml” which was created on Feb. 2, 2023 and is 6,937 bytes. The entire content of the sequence listing is incorporated herein by reference in its entirety.

### BACKGROUND OF THE INVENTION

Among all the bacteria species, infections from the gram-positive bacteria, such as Staphylococcus aureus, remain as the major threat to humans due to their severe virulence. New drugs are needed to combat these bacterial infections, especially drugs effect against multidrug-resistant pathogens¹. Apart from the crisis of antibiotics resistance, adding to the challenge is antibiotics tolerance, including difficult-to-treat persisters and biofilms. Such antibiotics tolerance plays a major role in chronic or recurrent bacterial infections².

Persisters have a distinct phenotype compared to the normal bacteria. Persisters are often slow growing or even growth arrested under the treatment of antibiotics; however, they are highly tolerant to the antibiotics. Once the antibiotics are removed, the persisters will resume their growth. The presence of the persisters gives rise to the recalcitrance and relapse of persistent bacterial infections, which also lead to the overuse of the antibiotics and enhance antibiotic resistance in bacteria. It is expected that the combination of anti-persister drugs and antibiotics could greatly improve the therapeutic effects of current anti-chronic bacterial infection treatments³.

Instead of growing in liquid medium, bacteria can also attach to solid surfaces to develop a hydrated polymeric network called a biofilm. In a biofilm, the environments are highly diverse in different regions, and there is high proportion of bacteria that are in a persister state. This accounts for the immense tolerance of biofilm bacteria to different classes of antibiotics⁴. Prolonged antibiotic treatment is often prescribed in anti-biofilm therapy, which leads to the deterioration of the antibiotics crisis⁵.

In bacteria, energy is generated in the form of ATP. Across the cell membrane, there is an electrochemical proton gradient called proton motive force (PMF), which is composed of a pH gradient (ΔpH) and a membrane potential gradient (Δψ). This allows for the transportation of other nutrients across the cell membrane and for the generation of ATP with a proton influx by ATP synthase⁶. In Streptococcus mutans, the PMF was dissipated when the RgpF gene was deleted. The mutant became less tolerant to stress⁷. Diarylquinolines, which target ATP synthase, were also able to eradicate dormant mycobacteria⁸. Protonophore CCCP was reported to inhibit biofilm formation in Pseudomonas aeruginosa⁹. Recently, it was reported that proton motive force is essential in Escherichia coli to mediate bacterial antibiotics tolerance¹⁰.

However, there remains a need for compounds effective against bacterial persister. Therefore, there is a need for development of the new compounds that target bacteria persister.

### BRIEF SUMMARY OF THE INVENTION

The subject invention pertains to novel compounds, compositions containing the novel compounds, and methods for targeting gram-positive bacteria with the compounds or compositions of the subject invention. Also disclosed are novel compounds, novel compositions, and methods for targeting gram-positive bacteria persister and/or for targeting gram-positive bacterial biofilms. The subject compounds and compositions can target gram-positive bacteria by modulating the proton motive force, calcium influx, autolysis, or any combination thereof. The novel compounds are synthetic, small molecules that can induce the death of gram-positive bacteria. In certain embodiments, the compounds are selective for one or more types of gram-positive bacteria, such as, for example, Staphylococcus aureus, Bacillus subtilis, Streptococcus pneumoniae, Listeria monocytogenes, Enterococcus faecalis or Mycobacterium smegmatis.

In certain embodiments, the compound is used for the treatment of infection of gram-positive bacteria. In certain embodiments, the compound is used for the treatment of an infection by, for example, Staphylococcus aureus, Bacillus subtilis, Streptococcus pneumoniae, Listeria monocytogenes, Enterococcus faecalis or Mycobacterium smegmatis. In certain embodiments, the compound is used for the treatment of a persister of, for example, Staphylococcus aureus, Bacillus subtilis, Streptococcus pneumoniae, Listeria monocytogenes, Enterococcus faecalis or Mycobacterium smegmatis. In certain embodiments, the compound is used for the treatment of a biofilm of, for example, Staphylococcus aureus, Bacillus subtilis, Streptococcus pneumoniae, Listeria monocytogenes, Enterococcus faecalis or Mycobacterium smegmatis.

In certain embodiments, the compound is used for the treatment of a gram-positive bacterial infection by inhibiting or altering the proton motive force in bacteria. In certain embodiments, the gram-positive bacteria can be targeted through various mechanisms, including, for example, membrane potential hyperpolarization, calcium ion influx, and accelerated autolysis.

Provided herein is a pharmaceutical composition comprising at least a compound of Formula (LVIII), a pharmaceutically acceptable salt, solvate or hydrate thereof, and at least one pharmaceutically acceptable excipient:

A-L₁-(B)ₙ-L₂-D. Formula (LVIII)

In certain embodiment, the pharmaceutical composition is used for the treatment of gram-positive bacteria. In certain embodiment, the bacterium is Staphylococcus aureus, Bacillus subtilis, Streptococcus pneumoniae, Listeria monocytogenes, Enterococcus faecalis or Mycobacterium smegmatis.

In certain embodiments, the pharmaceutical composition is used in combination with gentamicin.

In certain embodiments, the pharmaceutical product comprises the compounds of the subject invention and a distinct antibiotic in at least one composition for simultaneous administration or in at least two compositions for separate or staggered use as a medicament. In one embodiment, the pharmaceutical product is used for the treatment of gram-positive bacteria. In certain embodiment, the bacterium is Staphylococcus aureus, Bacillus subtilis, Streptococcus pneumoniae, Listeria monocytogenes, Enterococcus faecalis or Mycobacterium smegmatis.

In certain embodiments, the compounds and/or compositions of subject invention can be used in methods of treating a bacterial infection comprising the step of administering therapeutically effect amounts of the compounds disclosed herein to the subject in need thereof. The gram-positive bacteria include, for example, Staphylococcus aureus, Bacillus subtilis, Streptococcus pneumoniae, Listeria monocytogenes, Enterococcus faecalis or Mycobacterium smegmatis.

In certain embodiments, the gram-positive bacteria can be inhibited by, for example, inducing membrane hyperpolarization, Calcium influx, accelerated autolysis, or any combination thereof. In certain embodiments, the bacteria undergo accelerated autolysis. In certain embodiments, the compounds can, for example, suppress the virulence proteins secretion, eradicate bacterial persister, reduce established biofilms, inhibit biofilm formation, or any combination thereof. In certain embodiments, the compounds induce cell death of gram-positive bacteria over mammalian cells with up to 30-fold selectivity.

In certain embodiments, the subject invention further pertains to a kit comprising the compounds of the subject invention, compositions comprising the subject compounds and, optionally, other compounds, including, for example, antibiotics.

### BRIEF DESCRIPTION OF THE DRAWINGS

FIGS. 1A-1D are diagrams showing the chemical structure of synthetic compounds described in the example section.

FIGS. 2A-2B are the bar graphs showing the cytotoxicity of compound 8 (FIG. 2A) and compound 35 (FIG. 2B) towards mammalian cell NIH3T3, MDCK and MIHA. % survival is mean±S.D normalized to DMSO as the negative control of a representative experiment. Biological triplicates were conducted.

FIGS. 3A-3B are diagrams showing the hemolysis ability of compound 8 (FIG. 3A) and compound 35 (FIG. 3B) towards human red blood cell. % Hemolysis were mean±S.D of biological triplicates normalized to 0.1% triton X-100 as positive control.

FIG. 4 is a line graph showing the killing kinetics of the compound 8 and compound 35 towards Staphylococcus aureus persister compared to the conventional antibiotics. Staphylococcus aureus persister cells were challenged with 10×MIC of compound 8, compound 35, rifampin (RIF) (0.078 μg/mL), linezolid (LZD) (40 μg/mL), meropenem trihydrate (MEM) (2.5 μg/mL), daptomycin (DAP) (10 μg/mL), vancomycin hydrochloride (VAN) (10 μg/mL), gentamicin sulphate (GEN) (5 μg/mL) or negative control 1% DMSO. Data were representative of 3 independent experiments±S.D.

FIGS. 5A-5B are bar graphs showing the established Staphylococcus aureus biofilm treating with compound 8 (FIG. 5A) and compound 35 (FIG. 5B) in a concentration dependent manner are significantly reduced. 24 hours pre-established Staphylococcus. aureus ATCC 35556 biofilm was treated with various concentrations of compound 8, compound 35 or 1% DMSO as negative control for 24 hours. Biofilm was quantified using crystal violet. The data were normalized to DMSO (100% biofilm) and represent average values±S.D. (n=9 per group). Statistical analysis was performed by ANOVA with Dunnett's post-test to calculate p-values adjusted for multiple comparisons with DMSO as control condition (*p<0.05; **p<0.01; ***p<0.001).

FIGS. 6A-6B are bar graphs showing compound 8 (FIG. 6A) and compound 35 (FIG. 6B) can significantly inhibited the formation of the Staphylococcus aureus biofilm in a concentration dependent manner. Staphylococcus aureus ATCC 35556 was treated with various concentrations of compound 8, compound 35 or 1% DMSO as negative control for 24 hours. Biofilm was quantified using crystal violet. The data were normalized to DMSO (100% biofilm) and represent average values±S.D. (n=9 per group). Statistical analysis was performed by ANOVA with Dunnett's post-test to calculate p-values adjusted for multiple comparisons with DMSO as control condition (*p<0.05; **p<0.01; ***p<0.001).

FIG. 7 is a volcano plot showing compound 8 can reduce the secretion of the virulence proteins of Staphylococcus aureus. The volcano plots showed a log 2-fold change of protein levels in supernatant after treatment of Staphylococcus aureus ATCC 29213 cells with compound 8 (1.56 μM, 0.5-fold MIC) compared with DMSO treatment. The red dots represented proteins which were annotated as “Virulence” and “Secrete/extracellular” as subcellular location by UniprotKB. The curve is derived at FDR=0.05 and s0=0.1.

FIG. 8 is a line graph showing compound 8 and compound 35 rescue mice from Staphylococcus aureus bacteremia infection model. Statistical analysis was performed with a log rank test (Mantel Cox test). (*p<0.05).

FIG. 9 are the bar graphs showing compound 8 and compound 35 reduce bacteria loads in different organs of the mice infected by Staphylococcus aureus. Grubb's test was performed to test for outliers. Statistical analysis was performed with two-sided paired t test. (*p<0.05).

FIG. 10 is a bar graph showing compound 8 can acidify Staphylococcus aureus cytoplasm. Cytoplasm pH was monitored by pH sensitive fluorescent protein over time (s). 2 μM nigericin (Nig) and 2 μM valinomycin (Val) were used as positive control and 1% DMSO was used as negative control. Data represented the mean±S.D. of biological triplicates. Statistical analysis was performed by one-way ANOVA with Dunnett's post-test to calculate p-values adjusted for multiple comparisons with DMSO negative control (*p<0.05; **p<0.01; ***p<0.001).

FIG. 11 is a line graph showing compound 8 can depolarize Staphylococcus aureus cell membrane under sub-inhibitory concentration and hyperpolarize Staphylococcus aureus cell membrane under inhibitory concentration. Membrane potential was monitored by DiSc₃(5) over time (s). Extracellular buffer contained 10 mM HEPEs buffer, 5 mM glucose and 100 mM NaCl, pH 7.1. At t=100 s, 6 μL different concentration of compound 8 and 10 mM valinomycin (Vln) were added to 594 μL MRSA Mu3 pre-incubated with 0.4 μM DiSC₃(5). The fluorescence signal was further monitored for 500 s. Data represented the mean±S.D. of technical triplicates.

FIG. 12 is a line graph showing compound 8 can induce calcium influx into cytoplasm. The fluorescence of the Fluo-4-AM loaded in Staphylococcus aureus Mu3 was monitored for 10 minutes. The average of fluorescence in this period was assigned as F₀. At t=10 minutes, different concentration of compound 8 was added to bacteria medium and then the fluorescence was further monitored for 40 minutes. Calcium ion transporter ionomycin was used as the positive control. The fluorescence Fₜ was normalized to initial fluorescence F₀. Data represented the mean of biological triplicates.

FIG. 13 is a bar graph showing calcium channel blockers can antagonize the antibacterial effect of compound 8 on Staphylococcus aureus. Exponential phase Staphylococcus aureus Mu3 was treated with compound 8 (4×MIC) or combination with calcium channel blocker. Data were representative of mean±S.D. of four individual experiments with error bars. Statistical analysis was performed by one-way ANOVA with Dunnett's post-test to calculate p-values adjusted for multiple comparisons with initial bacteria inoculum as control condition (*p<0.05; **p<0.01; ***p<0.001).

FIGS. 14A-14B are the bar graphs showing the reduction of the optical density of the culture of Staphylococcus aureus treated with compound 8 (FIG. 14A) or compound 35 (FIG. 14B), indicating accelerated autolysis. Staphylococcus aureus ATCC 29213 in CAMH was challenged with 4×MIC compounds 8 or compound 35. OD₅₉₅ was measured after 24 hours. Data represented the mean±S.D. of biological triplicates. Statistical analysis was performed by unpaired t test in 95% confidence interval (*=p<0.05; **p<0.01; ***p<0.001; ****p<0.001).

FIG. 15 is the bar graph showing the reduction of bactericidal effect of compound 8 in autolysin deletion mutants of Staphylococcus aureus. Staphylococcus aureus ATCC29213 WT, Staphylococcus aureus ATCC 29213, Δatl, ΔSAOUHSC_02855 and ΔSAOUHSC_01895 was incubated with 4×MIC compounds 8 for 24 hours. 10 μL bacteria were serial diluted and spotted on Mueller Hinton Agar. Colonies were enumerated next day. Data represented the mean±S.D. of biological triplicates. Statistical analysis was performed by unpaired t test compared to wild type in 95% confidence interval (*p<0.05; **p<0.01).

FIGS. 16A-16B are the images showing the compound 8 and compound 35 can induced the cell burst of Staphylococcus aureus. (FIG. 16A) Cells are imaged under scanning electron microscope. (FIG. 16B) Cells are imaged under transmission electron microscope.

### BRIEF DESCRIPTION OF THE SEQUENCES

SEQ ID NO: 1: primer pGL-f
SEQ ID NO: 2: primer pGL-r
SEQ ID NO: 3: primer pHluorin-f
SEQ ID NO: 4: primer pHluorin-r
SEQ ID NO: 5: primer pHluorin-f2
SEQ ID NO: 6: primer pHluorin-r2
SEQ ID NO: 7: primer RKC0482

### DETAILED DISCLOSURE OF THE INVENTION

Gram-positive bacteria, especially Staphylococcus aureus are the difficult-to-treat pathogens threating the human mankind. Apart from the multi-drug resistant mutants, adding to the challenge is the presence of the antibiotics tolerance including persisters and biofilms, which lead to the chronic and recurrent bacterial infections. Provided herein is a method of treatment of gram-positive bacteria by, for example, inducing the death of the bacteria. Provided herein is a method of treatment of Staphylococcus aureus persister and biofilm. Provided herein are compounds bearing α-aminoxy acid units that induced the death of gram-positive bacteria, multidrug resistant bacteria, including, for example, Staphylococcus aureus, Bacillus subtilis, Streptococcus pneumoniae, Listeria monocytogenes, Enterococcus faecalis or Mycobacterium smegmatis a persister form of gram-positive bacteria, and/or a biofilm of gram-positive bacteria. α-Aminoxy acids are analogous to amino acids with an extra oxygen inserted into the amine and α-carbon unit; the compounds have extraordinary biostability.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues¹¹⁻¹³. The term nucleic acid is used interchangeably with gene, cDNA, and mRNA encoded by a gene.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the term “subject” refers to an animal, particularly a human. The term “subject” as used herein encompasses both human and non-human animals. The term “non-human animals” includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In some embodiments, the term “subject” refers to a mammal, including, but not limited to, murines, simians, humans, felines, canines, equines, bovines, mammalian farm animals, mammalian sport animals, and mammalian pets. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition thereof that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the terms “reducing”, “inhibiting”, “blocking”, “preventing”, “alleviating”, or “relieving” when referring to a compound, mean that the compound brings down the occurrence, severity, size, volume or associated symptoms of an infection by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the infection would normally exist without application of the compound or a composition comprising the compound.

“Pharmaceutically acceptable salt” refers to a salt of a compound of the invention that is pharmaceutically acceptable and that possesses the desired pharmacological activity of the parent compound. In particular, such salts that are non-toxic may be inorganic or organic acid addition salts and base addition salts.

“Pharmaceutically acceptable vehicle” refers to a diluent, adjuvant, excipient or carrier with which a compound of the invention is administered. A “pharmaceutically acceptable vehicle” refers to a substance that is non-toxic, biologically tolerable, and otherwise biologically suitable for administration to a subject, such as an inert substance, added to a pharmacological composition or otherwise used to facilitate administration of an agent and that is compatible therewith. Examples of vehicles include but are not limited to calcium carbonate, calcium phosphate, various sugars and types of starch, cellulose derivatives, gelatin, vegetable oils, and polyethylene glycols.

In some embodiments of the invention, the method comprises administration of multiple doses of the compositions of the subject invention. The method may comprise administration of therapeutically effective doses of a composition comprising the compound or composition thereof of the subject invention as described herein twice a day, once a day, every other day, three times a week, once a week, or a lower frequency. In some embodiments, doses are administered over the course of 1 week, 2 weeks, or more than 3 weeks. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays for detecting bacteria, which are known in the art. In some embodiments of the invention, the method comprises administration of the compositions at several time per day, including but not limiting to 2 times per day, 3 times per day, and 4 times per day.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Antibacterial Compounds

Provided herein are compounds that can induce the death of gram-positive bacteria. The compounds provided can rescue a subject from Staphylococcus aureus bacteremia. The compounds can selectively induce the death of bacteria by greater than about 1 to about 10, about 10 to about 20, about 20 to about 30, about 30 to about 40, about 40 to about 50, about 50 to about 60, about 60 to about 70, about 70 to about 80, about 80 to about 90, about 90 to about 100 fold selectivity over mammalian cell. In certain embodiments, the compounds can kill drug-resistant bacteria. In certain embodiments, the compounds induce bacteria death by the mechanism including, for example, the hyperpolarization of bacterial membrane potential, calcium influx, or acceleration of the bacteria autolysis. In certain embodiments, the compounds can disrupt cytoplasmic pH. In certain embodiments, the compound is a small synthetic molecule containing α-aminoxy acid scaffold.

In certain embodiments, the compound is formula (LVIII):

A-L₁-(B)₁-L₂-D Formula (LVIII)
wherein A is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, substituted or unsubstituted alkyl, substituted or unsubstituted alkenyl, substituted or unsubstituted alkynyl, substituted or unsubstituted C₃-C₂₀ cycloalkyl, or substituted or unsubstituted C₃-C₂₀ heterocyclyl; wherein each B independently has the structure:

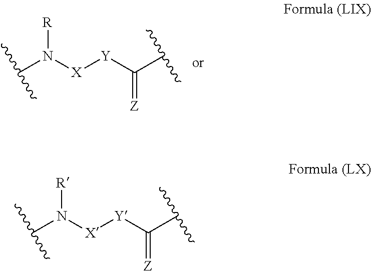

wherein X and X′ are independently O, absent, S, NR₂, substituted or unsubstituted alkyl, substituted or unsubstituted alkylene, or substituted or unsubstituted C₃-C₂₀ heterocycyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted aminoalkyl, substituted or unsubstituted aryl, substituted or unsubstituted alkaryl, substituted or unsubstituted aralkyl; wherein Y and Y′ are independently substituted or unsubstituted alkyl, absent, O, S, NR₃, substituted or unsubstituted alkylene, substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted heteroaralkyl, substituted or unsubstituted polyaralkyl, substituted or unsubstituted polyheteroaralkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocycyl, or substituted or unsubstituted aminoalkyl; wherein Z is O, S, or NR₄; wherein L₁ and L₂ are independently —OC(O)—, —C(O)—, —S(O)₂—, —O, absent, —C(O)O—, —S(O)—, —C(O)NH—, —C(O)NR^{iv}—, —NR^{iv}C(O)—, —C(O)OCH₂—, —SO₂NR^{iv}—, —CH₂R^{iv}—, —NR^{iv}H—, —NR^{iv}—, —OCONH—, —NHCOO—, —OCONR^{iv}—, —NR^{iv}COO—, —NHCONH—, —NR^{iv}CONH—, —NHCONR^{iv}—, —NRivCONR^{iv}—, —CHOH—, —CR^{iv}OH—, substituted or unsubstituted alkyl, substituted or unsubstituted alkylene, substituted or unsubstituted alkenyl, substituted or unsubstituted alkylamino, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl; wherein R₁, R₂, R₃, R₄, R₅, R, R′, R″, R′″, and R^{iv} are independently hydrogen, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted aralkyl, or substituted or unsubstituted acyl; wherein D is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, substituted or unsubstituted alkyl, substituted or unsubstituted alkenyl, substituted or unsubstituted alkynyl, substituted or unsubstituted C₃-C₂₀ cycloalkyl, substituted or unsubstituted C₃-C₂₀ cycloalkenyl, substituted or unsubstituted C3-C₂₀ cycloalkynyl, unsubstituted or substituted C₃-C₂₀ heterocyclyl, substituted or unsubstituted aminoalkyl, substituted or unsubstituted alkaryl, substituted or unsubstituted carboxyalkyl, substituted or unsubstituted alkoxycarbonyl, substituted or unsubstituted acyl, or substituted or unsubstituted aminocarbonyl, or H; and wherein n is an integer from 1 to 5.

In certain embodiments, A is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, or substituted or unsubstituted alkyl; and L₁ is —OC(O)—, —C(O)—, or —S(O)₂—.

In certain embodiments, A is a substituted or unsubstituted aryl, substituted or unsubstituted aralkyl, or substituted or unsubstituted alkyl, having the formula (R₇-)ₐ(aryl), (R₇-)ₐ(aralkyl), or (R₇-)ₐ(alkyl), respectively, wherein each R₇ is independently F₃C—, F—, Cl—, O₂N—, NC—, MeO—, HO—, HC(O)—, (R₈)₂N—, wherein each R₈ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted aralkyl, or substituted or unsubstituted acyl, and wherein a is 0, 1, 2, or 3.

In certain embodiments, A comprises an electron withdrawing group. In certain embodiments, the electron withdrawing group is CF₃, NO₂, F, Cl, Br, I, CN, CHO, or substituted or unsubstituted carbonyl, sulfonyl, trifluoroacetyl, or trifluoromethyl sulfonyl.

In certain embodiments, A is unsubstituted or substituted C₁₋₁₈ alkyl, substituted or unsubstituted aryl, or substituted or unsubstituted aralkyl, wherein the substituted C₁₋₁₈ alkyl, substituted aryl, or substituted aralkyl comprises an oxygen-, nitrogen-, or sulfur-containing moiety.

In certain embodiments, L₁ and L₂ are selected such that at least one of the atoms adjacent to B is oxygen, nitrogen, or sulfur.

In certain embodiments, X is substituted or unsubstituted heterocyclyl, wherein the heterocyclyl is optionally substituted with an oxygen-, nitrogen-, or sulfur-containing moiety, or an electron withdrawing group. In certain embodiments, the electron withdrawing group is CF₃, NO₂, F, Cl, Br, I, CN, or CHO.

In certain embodiments, each Y, Y′, or Y″ is independently substituted or unsubstituted C₁₋₁₀ alkyl, substituted or unsubstituted aralkyl, wherein the substituted alkyl or substituted aralkyl comprises an oxygen-, nitrogen-, or sulfur-containing moiety, or an electron withdrawing group. In certain embodiments, the electron withdrawing group is CF₃, NO₂, F, Cl, Br, I, CN, or CHO.

In certain embodiments, L₂ is NR₁₀, O, S, or absent, wherein R₁₀ is H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl, wherein D is H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C3-C₂₀ cycloalkynyl, substituted or unsubstituted C₃-C₂₀ heterocyclyl, aminoalkyl, substituted or unsubstituted aryl, substituted or unsubstituted alkaryl, substituted or unsubstituted C₃-C₂₀ aralkyl, substituted or unsubstituted carboxyalkyl, substituted or unsubstituted C₃-C₂₀ alkoxycarbonyl, substituted or unsubstituted C₃-C₂₀ acyl, or substituted or unsubstituted C₃-C₂₀ aminocarbonyl.

[symbol]

In certain embodiments, D is a substituted or unsubstituted aryl having the formula -(aryl)(-R₁₁)_{b}, —R₁₁, or —O—R₁₁, wherein each R₁₁ is independently —CF₃, —F, C—Cl, —NO₂, —CN, —O-Me, —OH, —NR₁₂, wherein each R₁₂ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl, wherein b is 0, 1, 2, or 3.

In certain embodiments, B is

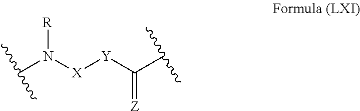

wherein X is O or absent, wherein Y is —CR₁₃R₁₄—, wherein R₁₃ is —CH₂—C₆H₅, —CH₂—O—C₆H₅, —C₁₋₄ alkyl, or —O—C₁₋₄ alkyl, wherein R₁₄ is H, wherein Z is O; wherein n is 1; wherein A has the formula (R₇-)ₐ(phenyl)-, L₁ is —C(O)—, wherein a is 2, wherein each R₇ is F₃C—; wherein D is —CH₂—C₆H₅, —CH₂—O—C₆H₅, —C₁₋₄ alkyl, or —O—C₁₋₄ alkyl; and wherein L₂ is NH, O, or S.

In certain embodiments, B is

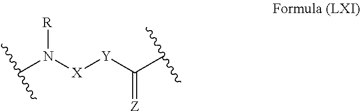

wherein X is O or absent, wherein Y is —CR₁₃R₁₄—, wherein R₁₃ is —CH₂—C₆H₅, —CH₂—O—C₆H₅, —C₁₋₄ alkyl, —O—C₁₋₄ alkyl, —CH₂-(phenyl)(-R₁₅)ₑ, —CH—O-(phenyl)(-R₁₅)ₑ, —C₁₋₄ alkyl-R₁₅, or —O—C₁₋₄ alkyl-R₁₅, wherein R₁₄ is H, wherein each R₁₅ is independently —CF₃, —F, C—Cl, —NO₂, —CN, —O-Me, —OH, —NH, wherein e is 1 or 2, wherein Z is O; wherein n is 1; wherein A is (R₇-)ₐ(phenyl)-CO—, C₆H₅—CH₂—, C₆H₅—O—CH₂—, C₁₋₄ alkyl-, or C₁₋₄ alkyl-O—, wherein a is 2, wherein each R₇ is independently F₃C—, F—, Cl—, O₂N—, NC—, MeO—, HO—, HN—; L₁ is —C(O)— or absent; wherein D is —CH₂—C₆H₅, —CH₂—O—C₆H₅, —C₁₋₄ alkyl, —O—C₁₋₄ alkyl, —CH₂-(phenyl)(-R₁₆)_{d}, —CH₂—O-(phenyl)(-R₁₆)_{d}, —C₁₋₄ alkyl-R₁₆, or —O—C₁₋₄ alkyl-R₁₆, wherein each R₁₆ is independently —CF₃, —F, C—Cl, —NO₂, —CN, —O-Me, —OH, —NH, wherein d is 1 or 2; and wherein L₂ is NH, O, or S.

In certain embodiments, the subject compounds can have the following structure, according to Formula (LXII), Formula (LXIII), or Formula (LXIV):

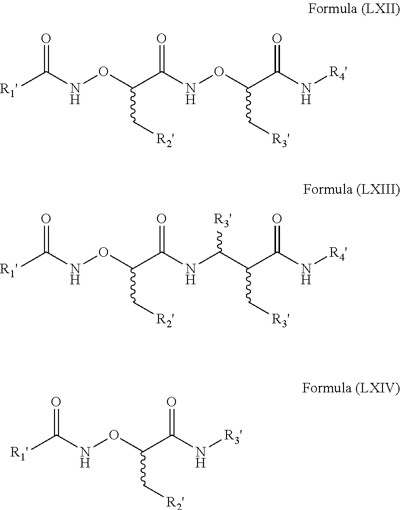

wherein each R₁′-R₅′ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl.

In certain embodiments, the compound that can target a gram-positive bacteria or a biofilm or persister thereof is according to Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XVII), Formula (XVIII), Formula (XXIX), Formula (XXX), Formula (XXXI), Formula (XXXII), Formula (XXXIII), Formula (XXXIV), Formula (XXXV), Formula (XXXVI), Formula (XXXVII), Formula (XXXVIII), Formula (XXXIX), Formula (XL), Formula (XLI), Formula (XLII), Formula (XLIII), Formula (XLIV), Formula (XLV), Formula (XLVI), Formula (XLVII), Formula (XLVIII), Formula (XLIX), Formula (L), Formula (LI), Formula (LII), Formula (XLIII), Formula (LIV), Formula (LV), Formula (LVI), or Formula (LVII):

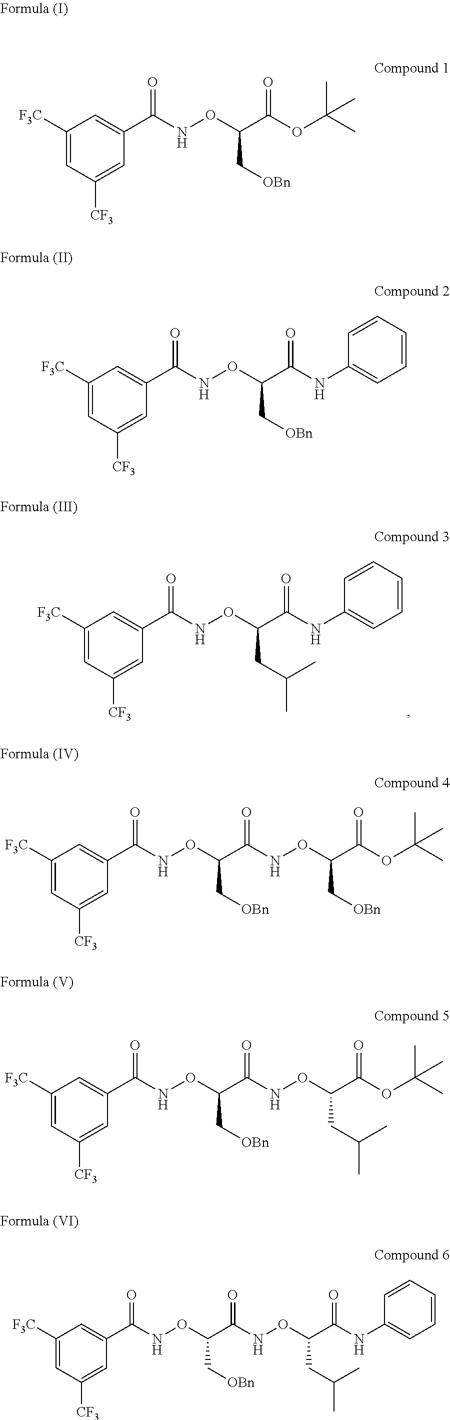 [SMILES: CC(C)(C)OC(=O)[C@@H](COCc1ccccc1)ONC(=O)c1cc(C(F)(F)F)cc(C(F)(F)F)c1 ; O=C(NO[C@H](COCc1ccccc1)C(=O)Nc1ccccc1)c1cc(C(F)(F)F)cc(C(F)(F)F)c1 ; CC(C)C[C@@H](ONC(=O)c1cc(C(F)(F)F)cc(C(F)(F)F)c1)C(=O)Nc1ccccc1 ; CC(C)(C)OC(=O)[C@@H](COCc1ccccc1)ONC(=O)[C@@H](COCc1ccccc1)ONC(=O)c1cc(C(F)(F)F)cc(C(F)(F)F)c1 ; CC(C)C[C@H](ONC(=O)[C@@H](COCc1ccccc1)ONC(=O)c1cc(C(F)(F)F)cc(C(F)(F)F)c1)C(=O)OC(C)(C)C ; CC(C)C[C@H](ONC(=O)[C@H](COCc1ccccc1)ONC(=O)c1cc(C(F)(F)F)cc(C(F)(F)F)c1)C(=O)Nc1ccccc1]

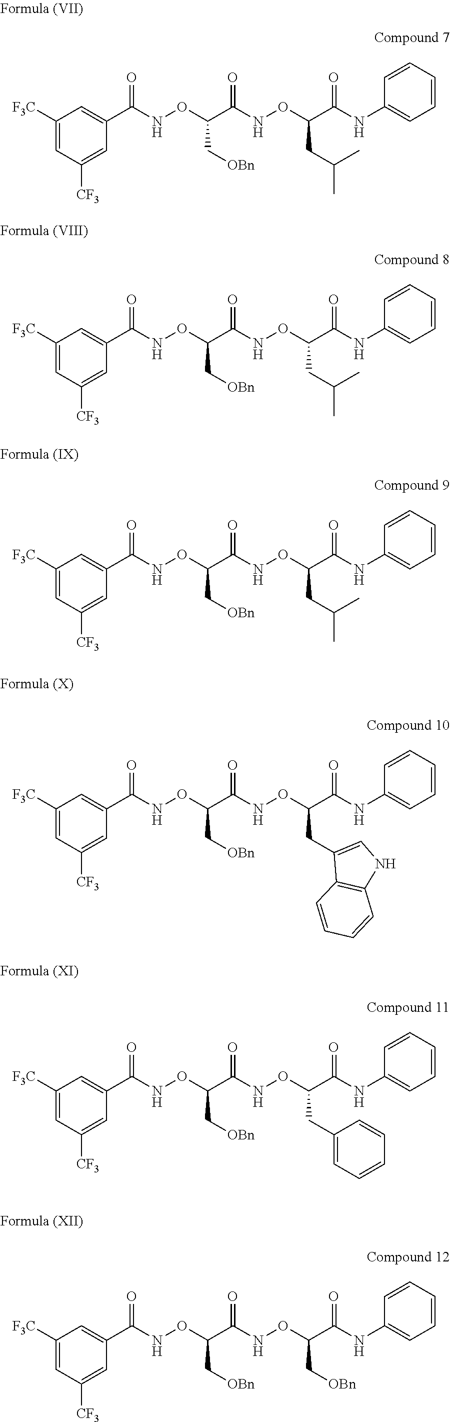

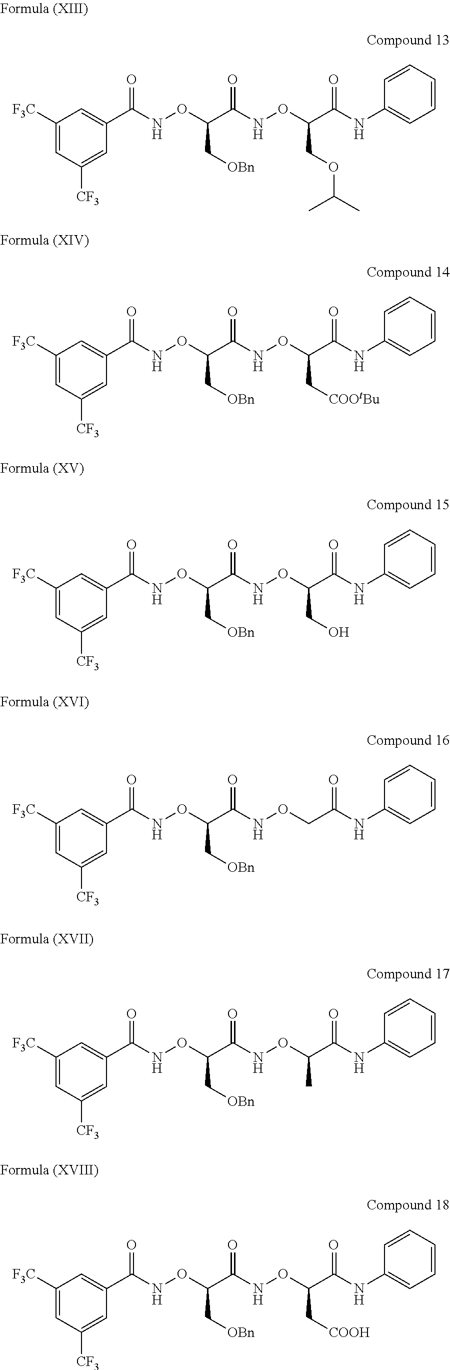

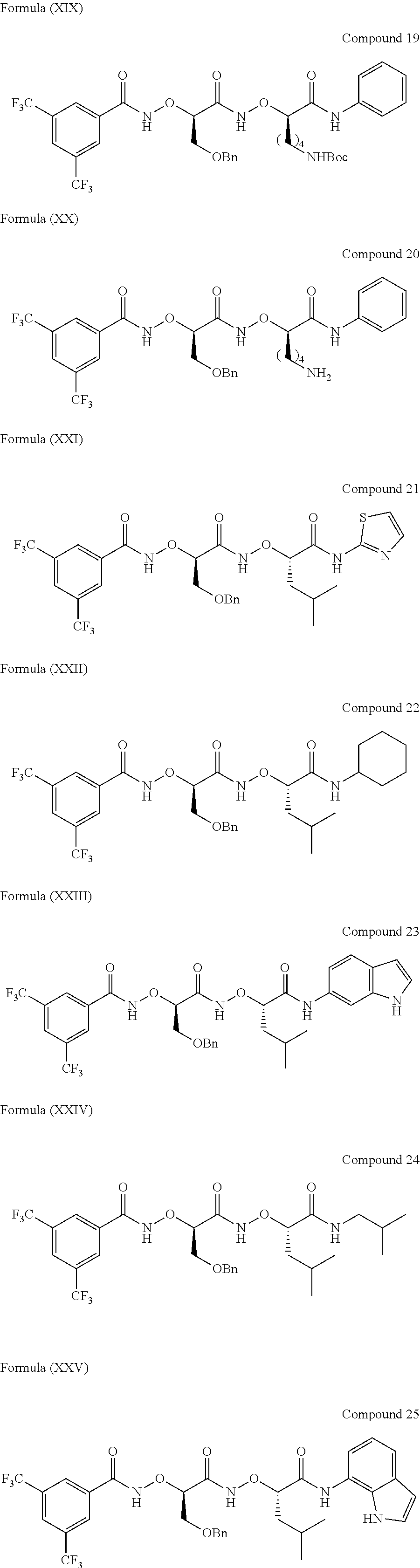

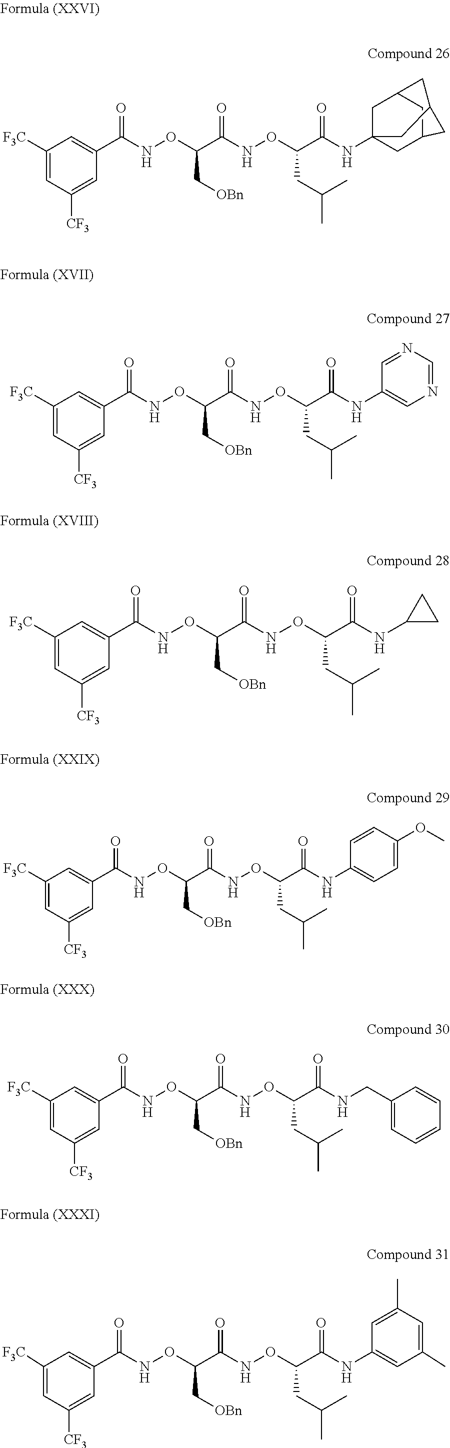

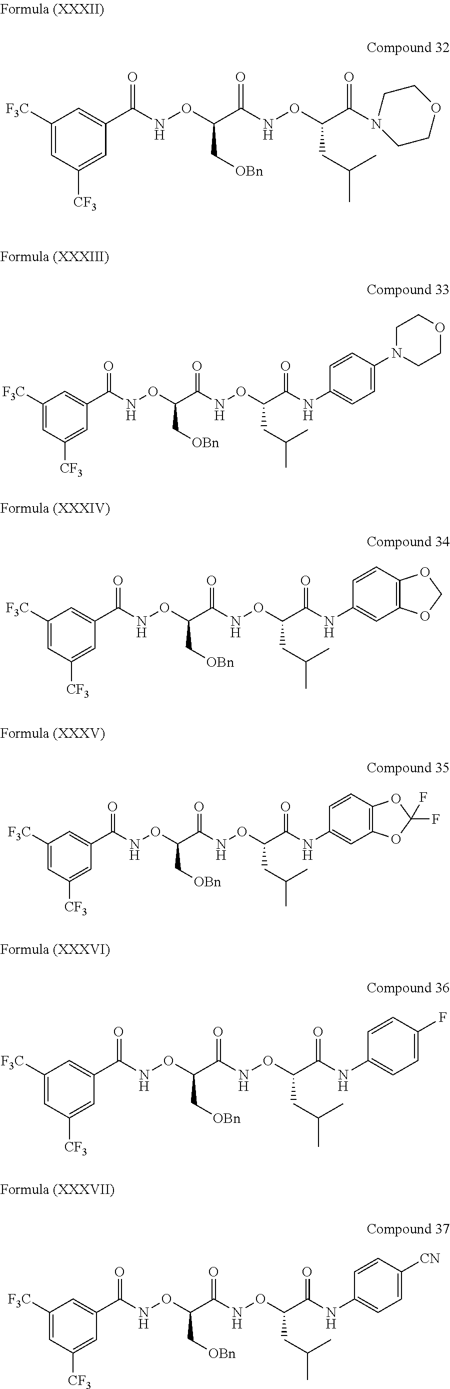

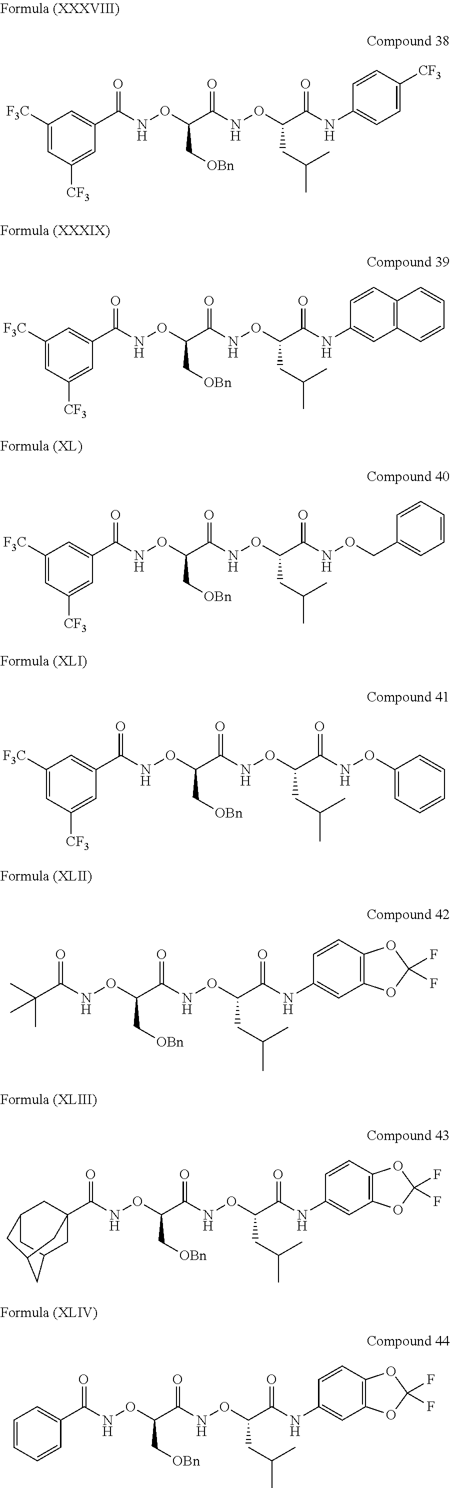

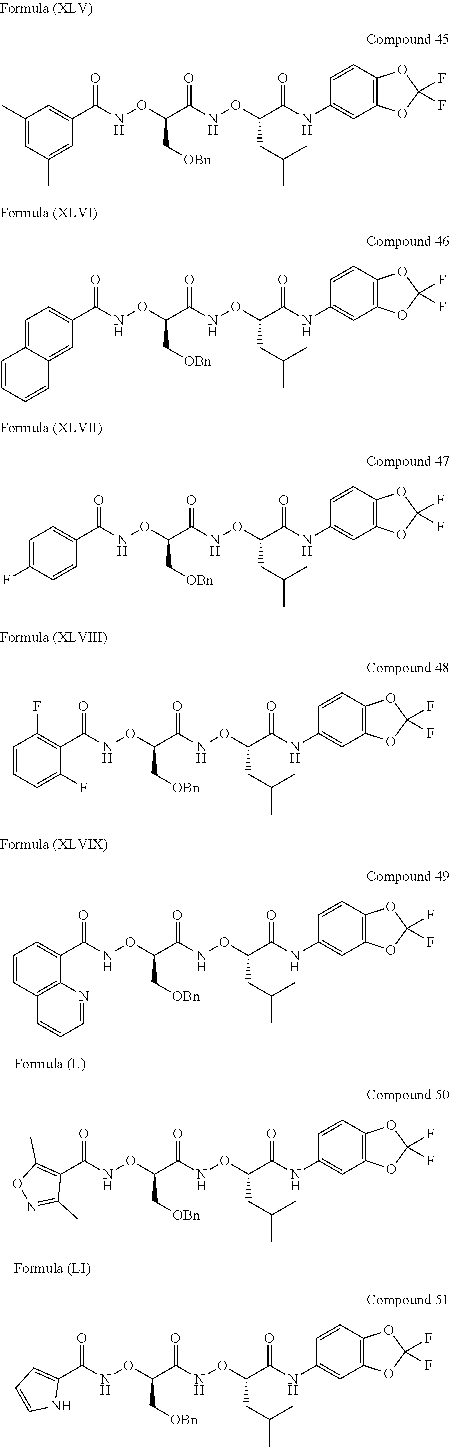

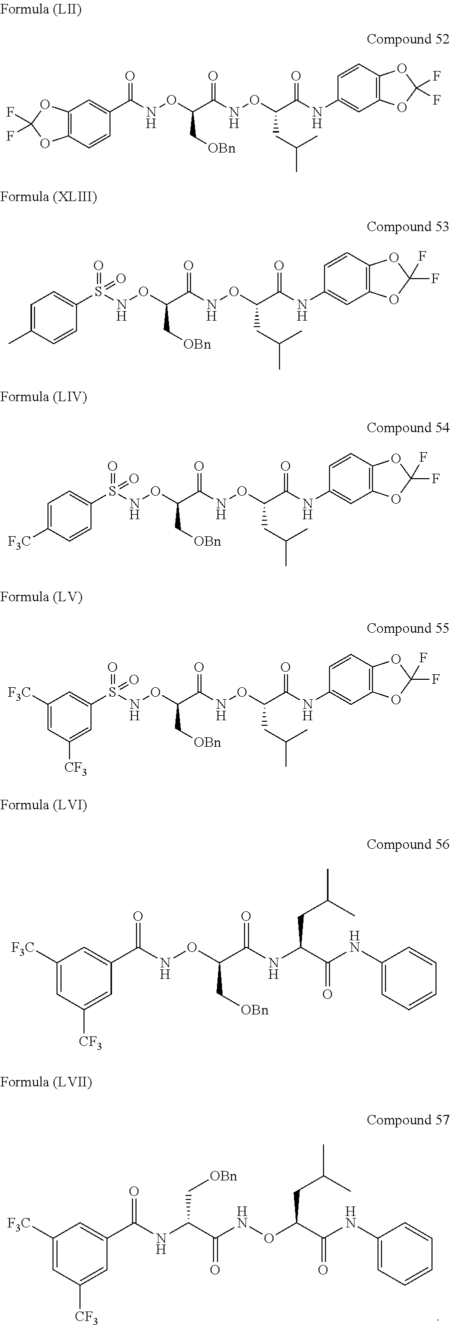

In certain embodiments, the compounds of the subject invention can be in a composition with gentamicin.

In some embodiments the subject invention provides pharmaceutically acceptable salts, solvates or hydrates thereof, of the compounds described herein.

Pharmaceutically acceptable salts can be a salt with an inorganic acid, such as hydrochloric acid, hydrobromic acid, perchloric acid, nitric acid, thiocyanic acid, sulfuric acid, and phosphoric acid; an organic acid, such as trifluoroacetic acid (TFA), formic acid, acetic acid, propionic acid, glycolic acid, lactic acid, pyruvic acid, oxalic acid, malonic acid, succinic acid, maleic acid, and fumaric acid; or a salt with a base, such as sodium hydroxide, ammonium hydroxide, potassium hydroxide, and organic bases such as mono-, di-, trialkyl and aryl amines, and substituted ethanolamines.

Further, pharmaceutically acceptable salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

Hydrate refers to a compound of the present invention or a salt thereof, that further includes a stoichiometric or non-stoichiometric amount of water bound by non-covalent intermolecular forces.

Solvate means a solvate formed from the association of one or more solvent molecules to a compound of the present invention. The term “solvate includes hydrates (e.g., mono hydrate, dihydrate, trihydrate, tetrahydrate, and the like).

Certain embodiments provide amorphous forms of salts of the compounds disclosed herein. Such amorphous forms are advantageous for oral, pulmonary, buccal, intravaginal, or suppository delivery.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The subject compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Synthesis of the Compounds

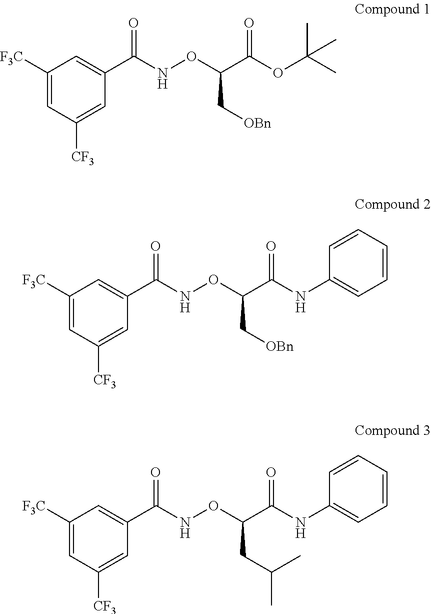 [SMILES: CC(C)(C)OC(=O)[C@@H](COCc1ccccc1)ONC(=O)c1cc(C(F)(F)F)cc(C(F)(F)F)c1 ; O=C(NO[C@H](COCc1ccccc1)C(=O)Nc1ccccc1)c1cc(C(F)(F)F)cc(C(F)(F)F)c1 ; CC(C)C[C@@H](ONC(=O)c1cc(C(F)(F)F)cc(C(F)(F)F)c1)C(=O)Nc1ccccc1]

Compounds 1, 2, and 3 were synthesized according to previously described methods¹³. Compound 1, compound 2 and compound 3 were obtained.

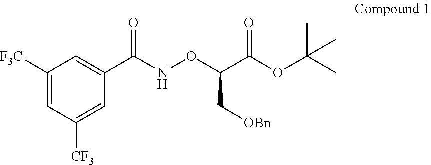 [SMILES: CC(C)(C)OC(=O)[C@@H](COCc1ccccc1)ONC(=O)c1cc(C(F)(F)F)cc(C(F)(F)F)c1]

Compound 1: ¹H NMR (500 MHz, CD₃CN) δ 10.17 (s, 1H), 8.24 (s, 2H), 8.16 (s, 1H), 7.36-7.29 (m, 5H), 4.63 (t, J=3.3 Hz, 1H), 4.57 (q, J=11.8 Hz, 2H), 3.93-3.87 (m, 2H), 1.46 (s, 9H); 13C NMR (125 MHz, CDCl3) δ 169.0, 163.4, 137.4, 133.8, 132.2 (q, ²J_{C—F}=34.0 Hz), 128.5, 128.0, 127.9, 127.6, 125.3, 123.0 (q, ¹J_{C—F}=271.3 Hz), 83.4, 77.4, 73.7, 69.2, 28.1; ¹⁹F (376 MHz, CDCl₃) δ −63.0; HRMS (ESI) for C₂₃H₂₃F₆NNaO₅ (M+Na⁺): calcd 530.1378, found 530.1395.

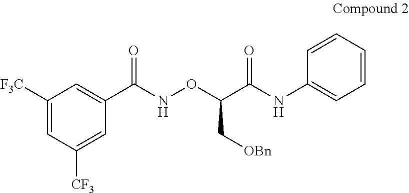 [SMILES: O=C(NO[C@H](COCc1ccccc1)C(=O)Nc1ccccc1)c1cc(C(F)(F)F)cc(C(F)(F)F)c1]

Compound 2: ¹H NMR (400 MHz, CDCl₃) δ 10.34 (br s, 2H), 8.17 (s, 2H), 8.02 (s, 1H), 7.66 (s, 1H), 7.65 (s, 1H) 7.34-7.25 (m, 7H), 7.12 (tt, J=7.4, 1.0 Hz, 1H), 4.70-4.68 (m, 1H), 4.62, 4.57 (AB_{q}, J_{AB}=11.8 Hz, 2H), 4.13 (dd, J=11.6, 2.5 Hz, 1H), 3.95 (dd, J=11.1, 8.2 Hz, 1H); 13C (125 MHz, CDCl₃) δ 166.3, 164.9, 137.4, 137.2, 132.8, 132.6 (q, 2 J_{C—F}=33.2 Hz), 129.1, 128.8, 128.4, 128.0, 127.8, 126.2, 125.0, 122.9 (q, J_{C—F}=273.1 Hz), 120.2, 87.2, 74.1, 70.1; ¹⁹F (376 MHz, CDCl₃) δ −62.9; HRMS (ESI) for C₂₅H₂₁F₆N₂O₄(M+H⁺): calcd 527.1400, found 527.1413.

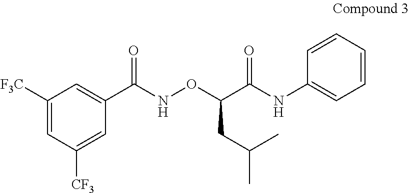 [SMILES: CC(C)C[C@@H](ONC(=O)c1cc(C(F)(F)F)cc(C(F)(F)F)c1)C(=O)Nc1ccccc1]

Compound 3: ¹H NMR (400 MHz, CD₃CN) δ 10.51 (s, 1H), 9.88 (s, 1H), 8.31 (s, 2H), 8.19 (s, 1H), 7.68 (d, J=8.0 Hz, 2H), 7.36-7.32 (m, 2H), 7.13-7.09 (m, 1H), 4.51 (dd, J=9.6, 3.8 Hz, 1H), 1.90-2.02 (m, 1H), 1.83-1.70 (m, 2H), 1.06 (d, J=6.6 Hz, 3H), 1.01 (d, J=6.6 Hz, 3H); ¹³C NMR (150 MHz, CD₃OD) δ 172.2, 164.8, 139.1, 135.2, 133.1 (q, ²J_{C—F}=22.5 Hz), 139.9, 129.0, 127.1, 125.7, 124.4 (q, ¹J_{C—F}=270.0 Hz), 121.3, 86.6, 42.0, 25.9, 23.6, 22.3; ¹⁹F (376 MHz, CD₃CN) δ −63.0; HRMS (ESI) for C₂₁H₂₀F₆N₂ NaO₃ (M+Na⁺): calcd 485.1276, found 485.1283.

Compound 4 were synthesized according to the general procedures described below (Scheme 1).

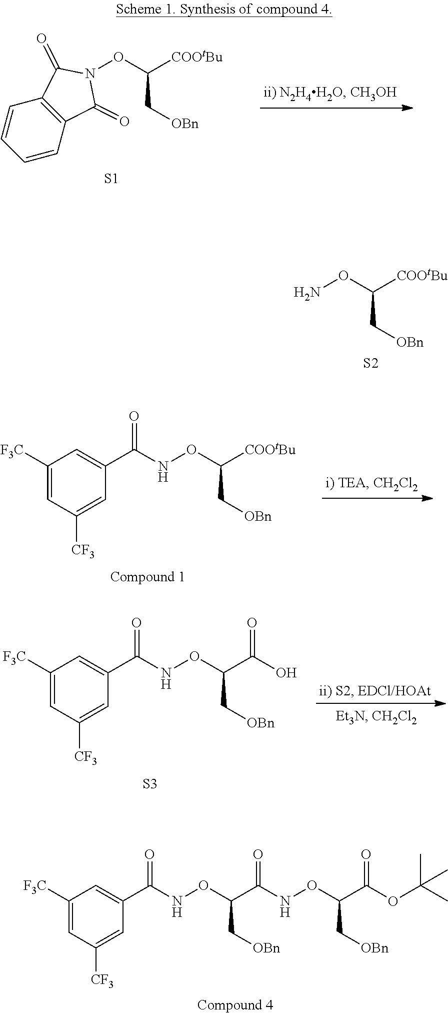

The starting material S1 was prepared according to the procedure described in literature¹³. To starting material S1 (1 g, 2.53 mmol) in CH₃OH solution (27 mL) was added N₂H₄·H₂O (378 mg, 7.56 mmol). After stirring for 1 hour at room temperature, the reaction mixture was concentrated under vacuo. The residue was dissolved in CH₂Cl₂ and filtered. The filtrated was concentrated to afford S2 as a colorless oil, which was used in subsequent reaction without further purification.

To a CH₂Cl₂ solution (4 mL) of compound 1 (996 mg, 2.0 mmol) was added trifluoroacetic acid (3 mL). The reaction mixture was stirred for 1 hour, then concentrated under vacuo and azeotroped with toluene 3 times to remove residual trifluoroacetic acid to afford free carboxylic acid S3. The reaction mixture was used in the subsequent reaction without further purification. To a CH₂Cl₂ solution (4 mL) of S3 (125 mg, 277 μmol) were added HOAt (41 mg, 302 μmol), trimethylamine (31 mg, 302 μmol) and EDCI·HCl (60 mg, 302 μmol) subsequently at 0° C. After the mixture was stirred for 10 minutes, S2 (100 mg, 252 μmol) was added dropwise at 0° C. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compounds 4. (95 mg, 2 steps yield 54%). R_{f}=0.4 (20% EtOAc in Hexane); ¹H NMR (400 MHz, CDCl₃) δ 11.28-9.71 (br, 1H), 8.19 (s, 2H), 7.96 (s, 1H), 7.33-7.21 (m, 11H), 4.76-4.66 (br, 1H), 4.63-4.33 (m, 5H), 4.04-3.77 (m, 4H), 1.40 (s, 9H); ¹³C NMR (100 MHz, CDCl₃) δ 168.2, 166.0, 163.8, 137.6, 137.2, 132.8, 132.2 (q, ²J_{C—F}=34.0 Hz), 128.7, 128.5, 128.2, 128.1, 127.90, 127.87, 127.4, 125.4, 123.0 (q, ¹J_{C—F}=272.9 Hz), 84.7, 83.6, 83.1, 74.0, 73.7, 69.3, 69.0, 28.0; ¹⁹F NMR (376 MHz, CD₃OD) δ −62.9; HRMS (ESI) for C₃₃H₃₅ F₆N₂O₈ (M+H)⁺: calculated 701.2292, found 701.2284.

Compound 5 was synthesized according to the general procedures described below (Scheme 2).

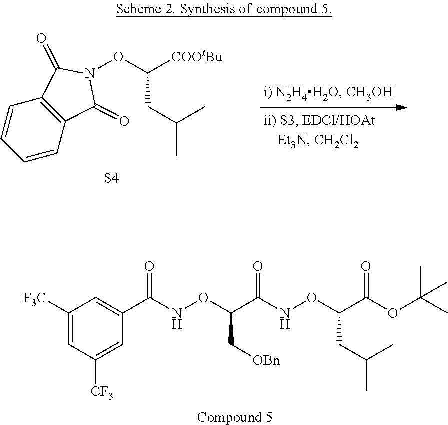

The starting material S4 was prepared according to the procedure described in literature¹³. To starting material S4 (100 mg, 0.3 mmol) in CH₃OH solution (2.7 mL) was added N₂H₄·H₂O (45 mg, 0.9 mmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To a CH₂Cl₂ solution (3 mL) of S3 (149 mg, 330 μmol) were added HOAt (49 mg, 360 μmol), trimethylamine (36 mg, 360 μmol) and EDCI·HCl (71 mg, 360 μmol) subsequently at 0° C. After the mixture was stirred for 10 minutes, the free amine was added dropwise at 0° C. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compounds 5. (159 mg, 83% yield for 2 steps). R_{f}=0.5 (20% EtOAc in Hexane); ¹H NMR (400 MHz, CDCl₃) δ 11.26 (s, 1H), 10.76 (s, 1H), 8.24 (s, 2H), 7.98 (s, 1H), 7.34-7.16 (m, 5H), 4.66 (s, 1H), 4.60-4.48 (m, 2H), 4.43-4.32 (m, 1H), 4.02-3.90 (m, 1H), 3.90-3.76 (m, 1H), 1.95-1.80 (m, 1H), 1.68 (ddd, J=14.1, 8.3, 5.8 Hz, 1H), 1.58-1.50 (m, 1H), 1.45 (s, 9H), 0.94 (d, J=6.4 Hz, 3H), 0.90 (d, J=6.6 Hz, 3H); ¹³C NMR (100 MHz, CDCl₃) δ 171.1, 166.1, 164.4, 137.2, 132.9, 132.3 (q, ²J_{C—F}=34.3 Hz), 128.6, 128.1, 128.0, 127.9, 125.3, 122.9 (q, ¹J_{C—F}=273.2 Hz), 86.0, 83.0, 82.6, 73.8, 69.5, 39.9, 28.0, 24.7, 23.0, 22.0; ¹⁹F NMR (376 MHz, CDCl₃) δ −62.9; HRMS (ESI) for C₂₉H₃₅F₆N₂O₇(M+H)⁺: calculated 637.2343, found 637.2337.

Compound 6, 7 and 8 was synthesized according to the general procedures described below (Scheme 3).

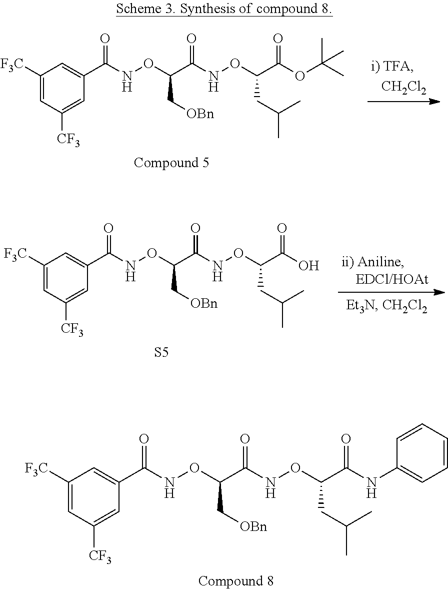 [SMILES: CC(C)C[C@H](ONC(=O)[C@@H](COCc1ccccc1)ONC(=O)c1cc(C(F)(F)F)cc(C(F)(F)F)c1)C(=O)OC(C)(C)C ; "Compound 5" ; CC(C)C[C@H](ONC(=O)[C@@H](COCc1ccccc1)ONC(=O)c1cc(C(F)(F)F)cc(C(F)(F)F)c1)C(=O)O ; "S5" ; CC(C)C[C@H](ONC(=O)[C@@H](COCc1ccccc1)ONC(=O)c1cc(C(F)(F)F)cc(C(F)(F)F)c1)C(=O)Nc1ccccc1 ; "Compound 8"]

To a CH₂Cl₂ solution (3 mL) of compound 5 (142 mg, 225 μmol) was added trifluoroacetic acid (3 mL). The reaction mixture was stirred for 1 hour, then concentrated under vacuo and azeotroped with toluene 3 times to remove residual trifluoroacetic acid to afford free carboxylic acid S5. The reaction mixture was used in the subsequent reaction without further purification. To the CH₂Cl₂ solution (2.2 mL) of S5 were added HOAt (40 mg, 292 μmol), trimethylamine (30 mg, 292 μmol) and EDCI·HCl (58 mg, 292 μmol) subsequently at 0° C. The reaction mixture was stirred for 10 minutes. Aniline (27 mg, 292 μmol) was added dropwise at 0° C. The resulting reaction mixture was stirred overnight, then washed sequentially with saturated NaHCO₃ solution and 0.1 M HCl solution. Then the organic layer was dried over anhydrous Na₂SO₄ and concentrated under vacuo. The reaction mixture was purified by flash chromatography to afford compound 8 as a white solid (64 mg, 43% yield for 2 steps). R_{f}=0.6 (30% EtOAc in Hexane); ¹H NMR (500 MHz, CD₃OD) δ 8.28 (s, 2H), 8.10 (s, 1H), 7.59 (d, J=7.9 Hz, 2H), 7.29-7.19 (m, 7H), 7.06 (t, J=7.4 Hz, 1H), 4.17-4.67 (m, 1H), 4.54-4.45 (m, 3H), 3.95-3.88 (m, 2H), 2.02-1.92 (m, 1H), 1.81 (ddd, J=14.4, 9.3, 5.2 Hz, 1H), 1.68 (ddd, J=14.3, 8.9, 4.1 Hz, 1H), 1.03 (d, J=6.6 Hz, 3H), 0.99 (d, J=6.7 Hz, 3H); ¹³C NMR (126 MHz, DMSO-d₆) δ 169.3, 166.0, 162.2, 138.4, 137.9, 133.8, 130.5 (q, ²J_{C—F}=33.2 Hz), 128.6, 128.2 (128.1), 127.6, 127.5, 125.4, 123.5, 123.0 (q, ¹J_{C—F}=273.1 Hz), 119.4, 84.1, 82.6, 72.4, 68.4, 24.3, 23.1, 21.8; ¹⁹F NMR (376 MHz, CD₃OD) δ −64.3 HRMS (ESI) for C₃₂H₃₂F₆N₃O₆ (M+H)⁺: calculated 656.2190, found 656.2176.

Compound 6, 7 and 9 were synthesized with the use of enantiomer and the diastereomer of compound 5.

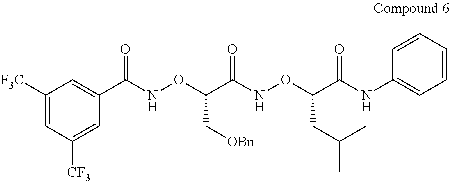 [SMILES: CC(C)C[C@H](ONC(=O)[C@H](COCc1ccccc1)ONC(=O)c1cc(C(F)(F)F)cc(C(F)(F)F)c1)C(=O)Nc1ccccc1]

Compound 6: ¹H NMR (500 MHz, CD₃OD) δ 8.23 (s, 2H), 8.09 (s, 1H), 7.57 (d, J=8.02 Hz, 2H), 7.28-7.14 (m, 7H), 7.01 (t, J=7.35 Hz, 1H), 4.73 (t, J=3.79 Hz, 1H), 4.53-4.43 (m, 3H), 3.93 (dd, J=11.1, 3.6 Hz, 1H), 3.89 (dd, J=10.8, 4.7 Hz, 1H), 1.97 (m, 1H), 1.79 (ddd, J=14.4, 8.6, 5.3 Hz, 1H), 1.69 (ddd, J=14.3, 8.6, 4.1 Hz 1H), 1.41 (s, 3H), 1.03 (d, J=6.6 Hz, 3H), 0.99 (d, J=6.6 Hz, 3H); ¹³C NMR (126 MHz, CDCl₃) δ 170.6, 168.0, 165.2, 137.3, 136.6, 132.4, 133.1 (q, J_{C—F}=33.3 Hz), 128.9, 128.6, 128.2, 128.0, 124.8, 122.8 (q, ²J_{C—F}=272.6 Hz), 120.4, 86.5, 86.4, 74.0, 69.4, 40.9, 25.0, 23.1, 21.7; ¹⁹F NMR (471 MHz, CDCl₃) δ −62.9; HRMS (ESI) for C₃₁H₃₁N₃O₆F₆ (M+H)⁺: calculated 656.2190, found 656.2184

[symbol]

Compound 10 was synthesized according to the general procedures described below (Scheme 4).

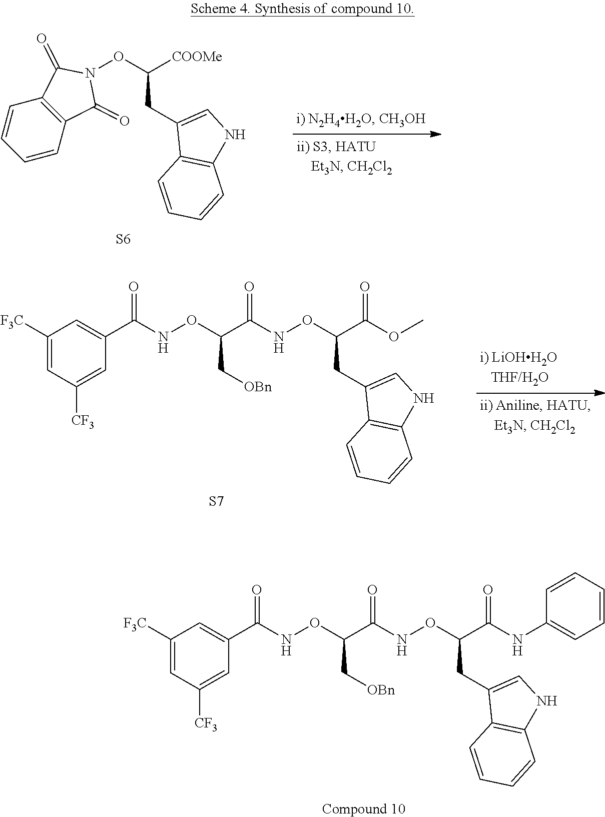 [SMILES: COC(=O)[C@@H](Cc1c[nH]c2ccccc12)ON1C(=O)c2ccccc2C1=O ; "S6" ; COC(=O)[C@@H](Cc1c[nH]c2ccccc12)ONC(=O)[C@@H](COCc1ccccc1)ONC(=O)c1cc(C(F)(F)F)cc(C(F)(F)F)c1 ; "S7" ; O=C(NO[C@H](COCc1ccccc1)C(=O)NO[C@H](Cc1c[nH]c2ccccc12)C(=O)Nc1ccccc1)c1cc(C(F)(F)F)cc(C(F)(F)F)c1 ; "Compound 10"]

The starting material S6 was prepared according to the procedure described in literature¹⁴. To starting material S6 (150 mg, 396 mmol) in CH₃OH solution (4 mL) was added N₂H₄·H₂O (61 mg, 1.22 mmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To the CH₂Cl₂ solution (5 mL) of S3 (178 mg, 396 μmol), HATU (151 mg, 396 μmol) and trimethylamine (40 mg, 396 μmol) were added and stirred for 30 minutes. 2.30 was subsequently added. The reaction was stirred for overnight and then concentrated under vacuo. The reaction mixture was purified by flash chromatography to afford compounds S7. (233 mg, 88% yield for 2 steps). R_{f}=0.5 (40% EtOAc in Hexane); ¹H NMR (500 MHz, CD₃OD) δ 8.33 (s, 2H), 8.15 (s, 1H), 7.52 (d, J=7.8 Hz, 1H), 7.36-7.21 (m, 6H), 7.16 (s, 1H), 7.07 (t, J=7.2 Hz, 1H), 6.98 (t, J=7.6 Hz, 1H), 4.75 (t, J=6.3 Hz, 1H), 4.70-4.64 (m, 1H), 4.59 (s, 2H), 3.95-3.87 (m, 2H), 3.51 (s, 3H) 3.35-3.32 (m, 2H); ¹³C NMR (126 MHz, CDCl₃) δ 171.8, 166.6, 164.2, 137.2, 135.9, 132.5, 132.0 (q, ²J_{C—F}=33.8 Hz), 128.6, 128.1, 127.9, 127.4, 127.2, 125.5, 122.8 (q, ¹J_{C—F}=273.4 Hz), 123.8, 121.8, 119.3, 118.2, 111.3, 108.9, 84.3, 83.8, 73.7, 69.0, 52.3, 26.8; ¹⁹F NMR (376 MHz, CD₃OD) δ −62.9; HRMS (ESI) for C₃₁H₂₈F₆N₃O₇ (M+H)⁺: calculated 668.1826, found 668.1812.

To the tetrahydrofuran/water (4/1) solution (5 mL) of compound S7 (90 mg, 135 μmol) was added LiOH·H₂O (20 mg, 472 μmol). The reaction was stirred for 1 hour. The reaction mixture was tuned to pH 4 and extracted with ethyl acetate 3 times. The organic layer was concentrated under vacuo to afford the free carboxylic acid as a white solid, which was used in subsequent reaction without further purification. To a CH₂Cl₂ solution (3 mL) of free carboxylic were added HATU (133 mg, 349 μmol) and trimethylamine (35 mg, 349 μmol). The resulting mixture was stirred for 30 minutes and aniline (33 mg, 349 μmol) was subsequently added. The reaction was stirred for overnight and then concentrated under vacuo. The crude mixture was purified by flash chromatography afford compound 10. (170 mg, 67% yield for 2 steps). R_{f}=0.3 (30% EtOAc in Hexane); ¹H NMR (500 MHz, CD₃OD) δ 8.22 (s, 2H), 8.09 (s, 1H), 7.63 (d, J=7.9 Hz, 1H), 7.44 (d, J=7.6 Hz, 2H), 7.30 (d, J=8.2 Hz, 1H), 7.24-7.14 (m, 8H), 7.05 (t, J=7.6 Hz, 1H), 7.00 (t, J=7.5 Hz, 1H), 6.96 (t, J=7.5 Hz, 1H), 4.71-4.67 (m, 2H), 4.49-4.43 (m, 2H), 3.91 (dd, J=11.0, 3.6 Hz, 1H), 3.87 (dd, J=11.0, 4.8 Hz, 1H), 3.44 (dd, J=15.0, 5.2 Hz, 1H), 3.37-3.32 (m, 1H); ¹³C NMR (126 MHz, MeOD) δ 169.4, 167.8, 164.8, 137.2, 136.6, 136.2, 132.0 (q, ²J_{C—F}=34.0 Hz), 132.1, 128.9, 128.5, 128.2, 127.9, 127.8, 127.4, 126.0, 124.7, 122.8 (q, ¹J_{C—F}=272.9 Hz), 123.4, 122.1, 120.3, 119.5, 118.9, 111.2, 110.3, 88.3, 85.2, 74.5, 69.8, 28.7; ¹⁹F NMR (376 MHz, CD₃OD) δ −64.4; HRMS (ESI) for C₃₆H₃₁F₆N₄O₆ (M+H)⁺: calculated 729.2142, found 729.2126.

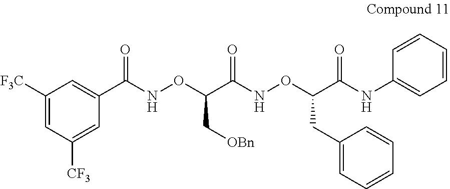 [SMILES: O=C(NO[C@H](COCc1ccccc1)C(=O)NO[C@@H](Cc1ccccc1)C(=O)Nc1ccccc1)c1cc(C(F)(F)F)cc(C(F)(F)F)c1]

Compound 11 was synthesized according to the literature¹². Compound 11: ¹H NMR (400 MHz, CDCl₃) δ 11.22 (br, 1H), 10.00 (s, 1H), 9.76 (br, 1H), 8.08 (s, 2H), 8.01 (s, 1H), 7.64 (s, 1H), 7.62 (s, 1H), 7.30-7.26 (m, 12H), 7.10-7.06 (m, 1H), 4.62-4.52 (m, 4H), 4.03 (d, J=11.9 Hz, 1H), 3.92-3.87 (m, 1H), 3.42 (d, J=13.62 Hz, 1H), 3.11-3.05 (m, 1H); ¹³C NMR (125 MHz, CD₃OD) δ 172.0, 170.4, 169.0, 138.9, 138.8, 137.5, 134.8, 133.0 (q, ²J_{C—F}=33.2 Hz), 130.7, 129.7, 129.4, 129.3, 128.9, 128.7, 128.6, 127.8, 126.1, 125.6, 124.4 (q, ¹J_{C—F}=272.3 Hz), 121.4, 88.3, 84.8, 74.5, 69.6, 38.8; ¹⁹F NMR (376.5 MHz, CDCl₃) δ −62.9; HRMS (ESI) for C₃₄H₃₀F₆N₃O₆ (M+H⁺): calcd 690.2033, found 690.2003.

Compound 12 was synthesized according to the general procedures described below (Scheme 5).

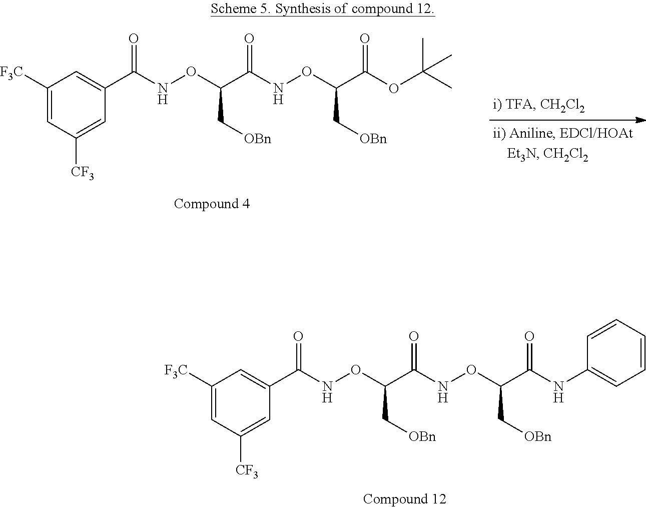 [SMILES: CC(C)(C)OC(=O)[C@@H](COCc1ccccc1)ONC(=O)[C@@H](COCc1ccccc1)ONC(=O)c1cc(C(F)(F)F)cc(C(F)(F)F)c1 ; "Compound 4" ; O=C(NO[C@H](COCc1ccccc1)C(=O)NO[C@H](COCc1ccccc1)C(=O)Nc1ccccc1)c1cc(C(F)(F)F)cc(C(F)(F)F)c1 ; "Compound 12"]

To a CH₂Cl₂ solution (2 mL) of compound 4 (76 mg, 109 μmol) was added trifluoroacetic acid (2 mL). The reaction mixture was stirred for 1 hour, then concentrated under vacuo and azeotroped with toluene 3 times to remove residual trifluoroacetic acid to afford free carboxylic acid. The reaction mixture was used in the subsequent reaction without further purification. To the CH₂Cl₂ solution (2 mL) of free carboxylic acid were added HOAt (19 mg, 141 μmol), trimethylamine (14 mg, 141 μmol) and EDCI·HCl (28 mg, 141 μmol) subsequently at 0° C. The reaction mixture was stirred for 10 minutes. Aniline (13 mg, 141 μmol) was added dropwise at 0° C. The resulting reaction mixture was stirred overnight, then washed sequentially with saturated NaHCO₃ solution and 0.1 M HCl solution. Then the organic layer was dried over anhydrous Na₂SO₄ and concentrated under vacuo. The reaction mixture was purified by flash chromatography to afford compound 12 as a white solid (22 mg, 29% yield for 2 steps). R_{f}=0.2 (20% EtOAc in Hexane); ¹H NMR (500 MHz, DMSO-d₆) δ 12.55 (br, 1H), 12.12 (br, 1H), 10.42 (s, 1H), 8.34 (s, 2H), 8.30 (s, 1H), 7.61 (d, J=8.0 Hz, 2H), 7.34-7.21 (m, 12H), 7.05 (t, J=7.3 Hz, 1H), 4.71-4.67 (m, 1H), 4.67-4.62 (m, 1H), 4.58-4.47 (m, 4H), 3.89-3.77 (m, 4H); ¹³C NMR (126 MHz, DMSO-d₆) δ 166.6 (169.0), 162.0, 138.2, 138.0, 137.9, 133.9, 130.5 (q, ²J_{C—F}=33.3 Hz), 128.7, 128.21 (128.20) (128.19), 127.9 (128.1), 127.54, 127.50, 125.3, 123.8, 123.0 (q, ³J_{C—F}=275.2 Hz), 119.4, 85.3, 82.4, 72.4, 72.3, 69.0, 68.4; ¹⁹F NMR (376 MHz, CD₃OD) δ −64.4; HRMS (ESI) for C₃₅H₃₂F₆N₃O₇ (M+H)⁺: calculated 720.2139, found 720.2127.

Compound 13 was synthesized according to the general procedures described below (Scheme 6).

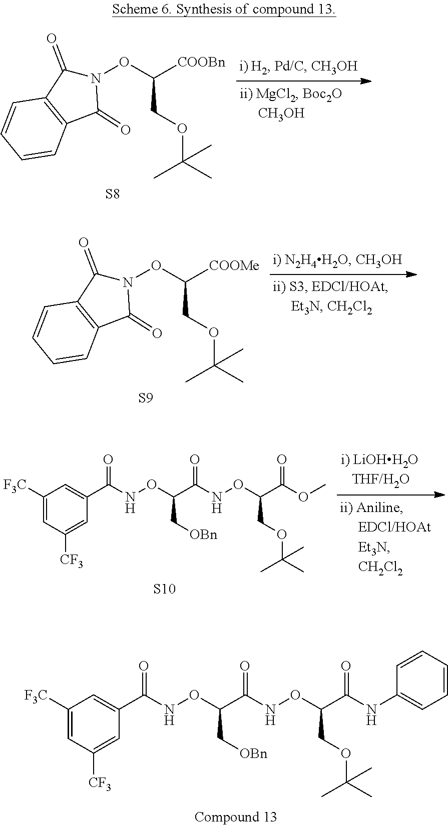

The starting material S8 was prepared according to the procedure described in literature⁵. To the methanol solution (25 mL) of compound S8 (907 mg, 2.28 mmol) was added 10% Pd/C. The reaction was stirred overnight under hydrogen balloon. The reaction mixture was filtered through celite and then rinsed with methanol. The filtrate was concentrated under vacuo to afford the free carboxylic acid as a white solid, which was used in subsequent reaction without further purification. To the methanol solution (1 mL) of free carboxylic acid were added MgCl₂·6H₂O (126.7 mg, 1.37 mmol) and di-tert-butyl dicarbonate (Boc₂O) (1 g, 4.57 mmol). The resulting mixture was stirred overnight and purified by flash chromatography to afford S9 as a colourless oil (359 mg, 49% yield for 2 steps). R_{f}=0.4 (20% EtOAc in Hexane); ¹H NMR (500 MHz, CDCl₃) δ 7.87-7.82 (m, 2H), 7.78-7.73 (m, 2H), 4.93 (t, J=5.0 Hz, 1H), 3.83 (dd, J=10.4, 6.3 Hz, 1H), 3.78 (dd, J=10.4, 4.1 Hz, 1H), 3.83 (s, 3H), 1.13 (s, 9H); ¹³C NMR (126 MHz, CDCl₃) δ 168.1, 163.1, 134.6, 129.0, 123.7, 84.7, 74.0, 61.5, 52.7, 27.2; HRMS (ESI) for C₁₆H₁₉NNaO₆ (M+Na)⁺: calculated 344.1105, found 344.1105.

To S9 (80 mg, 249 μmol) in CH₃OH solution (4 mL) was added N₂H₄·H₂O (37 mg, 747 μmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To a CH₂Cl₂ solution (3 mL) of S3 (135 mg, 300 μmol) were added HOAt (45 mg, 329 μmol), trimethylamine (33 mg, 329 μmol) and EDCI·HCl (65 mg, 329 μmol) subsequently at 0° C. After the mixture was stirred for 10 minutes, the free amine was added dropwise at 0° C. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compounds S10. (50 mg, 32% yield for 2 steps). R_{f}=0.5 (50% EtOAc in Hexane); ¹H NMR (500 MHz, CD₃OD) δ 8.34 (s, 2H), 8.15 (s, 1H), 7.36-7.22 (m, 5H), 4.73-4.65 (br, 1H), 4.60-4.57 (br, 2H), 4.56 (dd, J=4.4, 3.5 Hz, 1H), 3.94 (dd, J=10.9, 4.0 Hz, 1H), 3.91 (dd, J=11.0, 5.0 Hz, 1H), 3.82 (dd, J=10.5, 3.5 Hz, 1H), 3.75 (dd, J=10.5, 4.4 Hz, 1H), 3.70 (s, 3H), 1.14 (s, 9H); ¹³C NMR (126 MHz, CD₃OD) δ 171.0, 167.8, (167.2), 139.1, 135.0, 133.1 (q, ²J_{C—F}=33.9 Hz), 129.3, 128.9, 128.8, 126.3, 124.4 (q, ¹J_{C—F}=272.1 Hz), 84.6 (84.9), 74.8, 74.6, 69.9, 62.0, 52.6, 27.5; ¹⁹F NMR (376 MHz, CD₃OD) δ −64.4. HRMS (ESI) for C₂₇H₃₁N₂O₈F₆ (M+H)⁺: calculated 625.1979, found 625.1979.

To the tetrahydrofuran/water (4/1) solution (1 mL) of compound S10 (45 mg, 72 μmol) was added LiOH·H₂O (16.6 mg, 396 μmol). The reaction was stirred for 1 hour. The reaction mixture was tuned to pH 4 and extracted with ethyl acetate 3 times. The organic layer was concentrated under vacuo to afford the free carboxylic acid as a white solid, which was used in subsequent reaction without further purification. To a CH₂Cl₂ solution (2 mL) of free carboxylic acid were added HOAt (12 mg, 86 μmol), trimethylamine (9 mg, 329 μmol) and EDCI·HCl (17 mg, 86 μmol) subsequently at 0° C. After the mixture was stirred for 10 minutes, aniline was added dropwise (8 mg, 86 μmol) at 0° C. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compound 13 (34 mg, 48% yield for 2 steps) as a white solid. R_{f}=0.5 (30% EtOAc in Hexane); ¹H NMR (500 MHz, CD₃OD) δ 8.29 (s, 2H), 8.13 (s, 1H), 7.62-7.58 (m, 2H), 7.29-7.18 (m, 7H), 7.40 (t, J=7.4 Hz, 1H), 4.75 (t, J=7.5 Hz, 1H), 4.55-4.46 (m, 3H), 3.97 (dd, J=11.1, 3.8 Hz, 1H), 3.93 (dd, J=11.1, 4.8 Hz, 1H), 3.89 (dd, J=10.7, 3.1 Hz, 1H), 3.80 (dd, J=10.6, 5.7 Hz, 1H), 1.19 (s, 9H); ¹³C NMR (150 MHz, DMSO-d₆) δ 166.8, 166.2, 162.1, 138.3, 137.9, 133.9, 130.6 (q, ²J_{C—F}=33.3 Hz), 128.7, 128.2 (128.1), 127.55, 127.51, 125.6, 123.7, 123.0 (q, ¹J_{C—F}=273.0 Hz), 119.4, 85.8, 82.6, 73.0, 72.4, 68.4, 61.2, 27.1; ¹⁹F NMR (376 MHz, DMSO-d₆) δ −64.4; HRMS (ESI) for C₃₂H₃₄N₃O₇F₆ (M+H)⁺: calculated 686.2295, found 686.2292.

Compound 14 was synthesized according to the general procedures described below (Scheme 7).

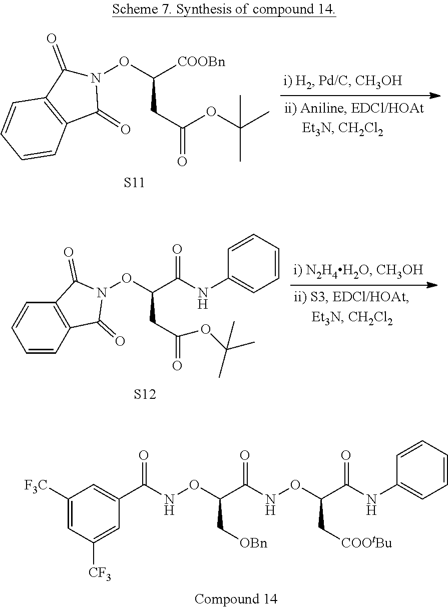

The starting material S11 was prepared according to the procedure described in literature⁵. To the methanol solution (7 mL) of compound S11 (300 mg, 705 μmol) was added 10% Pd/C. The reaction was stirred overnight under hydrogen balloon. The reaction mixture was filtered through Celite and then rinsed with methanol. The filtrate was concentrated under vacuo to afford the free carboxylic acid as a white solid, which was used in subsequent reaction without further purification. To a CH₂Cl₂ solution (3 mL) of S3 (109 mg, 323 μmol) were added HOAt (53 mg, 388 μmol), trimethylamine (39 mg, 389 μmol) and EDCI·HCl (77 mg, 388 μmol) subsequently at 0° C. After the mixture was stirred for 10 minutes, the aniline (33 mg, 355 μmol) was added dropwise at 0° C.

The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compounds S12 (18 mg, 14% yield for 2 steps). R_{f}=0.4 (20% EtOAc in Hexane); ¹H NMR (300 MHz, CDCl₃) δ 9.75 (s, 1H), 7.93-7.78 (m, 4H), 7.75 (d, J=8.1 Hz, 2H), 7.37 (t, J=7.9 Hz, 2H), 7.14 (t, J=7.1 Hz, 1H), 5.03 (dd, J=6.5, 4.4 Hz, 1H), 3.30 (dd, J=16.9, 4.5 Hz, 1H), 3.15 (dd, J=16.9, 6.1 Hz, 1H), 1.46 (s, 9H); ¹³C NMR (125 MHz, CDCl₃) δ 168.8, 166.3, 164.0, 137.7, 135.3, 129.1, 128.7, 124.7, 124.3, 120.0, 85.7, 81.9, 38.2, 28.1; HRMS (ESI) for C₂₂H₂₃N₂Na O₆ (M+Na)⁺: calculated 433.1370, found 433.1369.

To CH₃OH solution (0.5 mL) of compound S12 was added N₂H₄·H₂O (6.6 mg, 132 μmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To a CH₂Cl₂ solution (0.3 mL) of S3 (24 mg, 53 μmol) were added HOAt (7 mg, 53 μmol), trimethylamine (5 mg, 53 μmol) and EDCI·HCl (10 mg, 53 μmol) subsequently at 0° C. After the mixture was stirred for 10 minutes, the free amine was added dropwise at 0° C. The resulting reaction mixture was stirred overnight. The reaction mixture was washed with saturated NaHCO₃ solution and 0.1 M HCl aqueous solution subsequently. The reaction mixture was purified by flash chromatography to afford compound 14 (10 mg, 30% yield for 2 steps). R_{f}=0.2 (20% EtOAc in Hexane); ¹H NMR (500 MHz, CD₃OD) δ 8.31 (s, 2H), 8.17 (s, 1H), 7.62 (d, J=7.8 Hz, 2H), 7.31-7.18 (m, 7H), 7.12-7.03 (br, 1H), 4.79-4.71 (br, 2H), 4.55 (d, J=11.9 Hz, 1H), 4.50 (d, J=11.8 Hz, 1H), 4.00-3.91 (m, 2H), 3.00-2.92 (m, 2H), 2.85 (dd, J=16.6, 7.4 Hz, 1H), 1.46 (s, 9H); ¹³C NMR (126 MHz, CD₃OD) δ 168.4, 167.2, 166.8, 162.3, 138.3, 137.9, 134.1, 130.5 (q, ²J_{C—F}=33.3 Hz), 128.7, 128.2, 128.0, 127.52 (127.49), 125.3, 123.7, 123.0 (q, ¹J_{C—F}=273.0 Hz), 119.2, 82.9, 82.6, 80.5, 72.4, 68.5, 37.1, 27.6; ¹⁹F NMR (376 MHz, CD₃OD) δ −61.4; HRMS (ESI) for C₃₃H₃₄N₃O₈F₆ (M+H)⁺: calculated 714.2245, found 714.2245.

Compound 15 was synthesized according to the general procedures described below (Scheme 8).

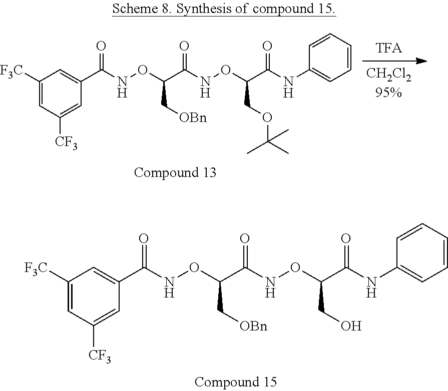 [SMILES: CC(C)(C)OC[C@@H](ONC(=O)[C@@H](COCc1ccccc1)ONC(=O)c1cc(C(F)(F)F)cc(C(F)(F)F)c1)C(=O)Nc1ccccc1 ; O=C(NO[C@H](COCc1ccccc1)C(=O)NO[C@H](CO)C(=O)Nc1ccccc1)c1cc(C(F)(F)F)cc(C(F)(F)F)c1]

To a CH₂Cl₂ solution (0.5 mL) of compound 13 (9 mg, 13 μmol) was added trifluoroacetic acid (0.5 mL) dropwise. The reaction mixture was stirred for 1 hour, then concentrated under vacuo and azeotroped with toluene 3 times to remove residual trifluoroacetic acid to afford compound 15 (8 mg, 95%). R_{f}=0.3 (50% EtOAc in Hexane); ¹H NMR (500 MHz, CD₃CN) δ 12.25-11.28 (br, 1H), 11.28-10.31 (br, 1H), 10.06 (s, 1H), 8.28 (s, 2H), 8.19 (s, 1H), 7.67-7.62 (m, 2H), 7.35-7.23 (m, 7H), 7.40 (tt, J=7.4, 1.1 Hz, 1H), 4.74 (dd, J=4.7, 3.1 Hz, 1H), 4.56-4.47 (m, 2H), 4.42 (dd, J=6.2, 3.1 Hz, 1H), 3.99-3.85 (m, 4H), 3.73-3.48 (br, 1H); ¹³C NMR (151 MHz, DMSO-d₆) δ 167.1, 166.7, 162.0, 138.3, 137.9, 134.1, 130.5 (q, ²J_{C—F}=33.3 Hz), 128.6, 128.2, 127.9, 127.53, 127.49, 125.2, 123.6, 123.0 (q, ¹J_{C—F}=273.3 Hz), 119.3, 86.3, 82.4, 72.4, 68.4, 60.8; ¹⁹F NMR (376 MHz, CD₃OD) δ −63.5; HRMS (ESI) for C₂₈H₂₆N₃O₇F₆ (M+H)⁺: calculated 630.1669, found 630.1664.

Compound 16 was synthesized according to the general procedures described below (Scheme 9).

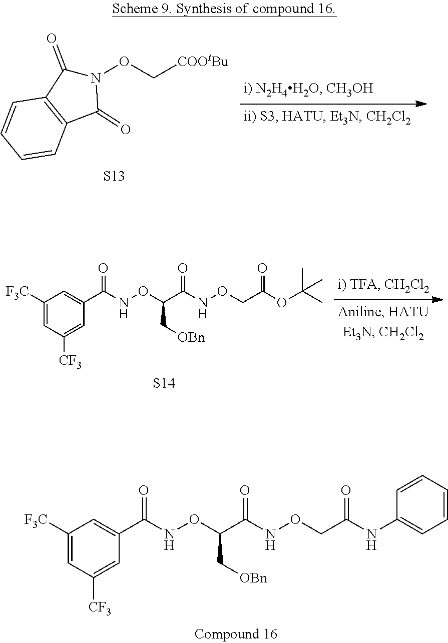

The starting material S13 was prepared according to the procedure described in literature¹³. To starting material S13 (47 mg, 170 μmol) in CH₃OH solution (1 mL) was added N₂H₄·H₂O (26 mg, 511 μmol). After stirring for 1 hour at room temperature, the reaction mixture was concentrated under vacuo. The residue was dissolved in CH₂Cl₂ and filtered. The filtrated was concentrated to afford the free amine as colorless oil, which was used in subsequent reaction without further purification.

To a CH₂Cl₂ solution (1 mL) of S3 (60 mg, 131 μmol) were added HATU (52 mg, 136 μmol) and trimethylamine (14 mg, 136 μmol). After the mixture was stirred for 30 minutes, the free amine was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compound S14. (65 mg, 2 steps yield 86%). R_{f}=0.5 (40% EtOAc in Hexane); ¹H NMR (500 MHz, CDCl₃) δ 11.91-11.06 (br, 1H), 11.06-9.82 (br, 1H), 8.23 (s, 2H), 7.98 (s, 1H), 7.33-7.20 (m, 5H), 4.75-4.64 (br, 1H), 4.60-4.50 (m, 2H), 4.41-4.30 (m, 2H), 3.99 (dd, J=11.0, 2.6 Hz, 1H), 3.89 (dd, J=11.0, 6.5 Hz, 1H), 1.44 (s, 9H); ¹³C NMR (126 MHz, CDCl₃) δ 168.2, 166.3, 164.6, 137.2, 132.1 (q, J=33.7 Hz), 128.6, 128.2, 127.9, 127.7, 125.6, 126.3-126.1 (m due to C—F splitting), 122.9 (q, J=274.1 Hz), 85.8, 83.0, 73.8, 72.9, 69.3, 28.0; ¹⁹F NMR (471 MHz, CD₃OD) δ −63.0; HRMS (ESI) for C₂₅H₂₇F₆N₂O₇ (M+H)⁺: calculated 581.1717, found 581.1716.

To a CH₂Cl₂ solution (0.5 mL) of compound S14 (65 mg, 113 μmol) was added trifluoroacetic acid (0.5 mL). The reaction mixture was stirred for 1 hour, then concentrated under vacuo and azeotroped with toluene 3 times to remove residual trifluoroacetic acid to afford free carboxylic acid. The reaction mixture was used in the subsequent reaction without further purification. To the CH₂Cl₂ solution (1 mL) of carboxylic acid added HATU (56 mg, 147 μmol) and trimethylamine (15 mg, 147 μmol). After the mixture was stirred for 30 minutes, aniline (14 mg, 147 μmol) was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compound 16. (50 mg, 73% yield for 2 steps). R_{f}=0.6 (40% EtOAc in Hexane); ¹H NMR (500 MHz, (CD₃)₂CO) δ 12.3-11.5 (br, 1H), 10.49 (s, 1H), 8.43 (s, 2H), 8.26 (s, 1H), 7.73 (d, J=7.7 Hz, 2H), 7.34-7.21 (m, 7H), 7.07 (t, J=7.4 Hz, 1H), 4.91 (dd, J=5.2, 3.1 Hz, 1H), 4.60-4.48 (m, 4H), 4.03 (dd, J=11.2, 3.1 Hz, 1H), 3.98 (dd, J=11.2, 5.5 Hz, 1H); ¹³C NMR (151 MHz, CDCl₃) δ 168.2, 167.3, 165.2, 137.2, 137.0, 132.5 (q, J=33.9 Hz), 132.3, 129.0, 128.7, 128.3, 128.0, 127.9, 126.3, 124.9, 123.0 (q, J=272.6 Hz), 120.3, 86.7, 76.5, 74.0, 69.4; ¹⁹F NMR (471 MHz, (CD₃)₂CO) δ −63.4; HRMS (ESI) for C₂₇H₂₄F₆N₃O₆ (M+H)⁺: calculated 600.1564, found 600.1564.

Compound 17 was synthesized according to the general procedures described below (Scheme 10).

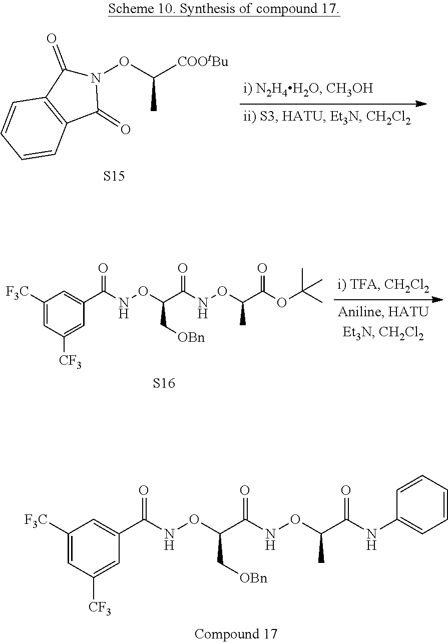

The starting material S15 was prepared according to the procedure described in literature¹³. To starting material S15 (50 mg, 170 μmol) in CH₃OH solution (1 mL) was added N₂H₄·H₂O (26 mg, 511 μmol). After stirring for 1 hour at room temperature, the reaction mixture was concentrated under vacuo. The residue was dissolved in CH₂Cl₂ and filtered. The filtrated was concentrated to afford the free amine as colorless oil, which was used in subsequent reaction without further purification.

To a CH₂Cl₂ solution (1 mL) of S3 (60 mg, 131 μmol) were added HATU (52 mg, 136 μmol) and trimethylamine (14 mg, 136 μmol). After the mixture was stirred for 30 minutes, the free amine was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compound S16. (7 mg, 2 steps yield 7%). R_{f}=0.5 (40% EtOAc in Hexane); ¹H NMR (500 MHz, (CD₃)₂CO) δ 8.46 (s, 2H), 8.26 (s, 1H), 7.39-7.20 (m, 4H), 7.27 (t, J=7.3 Hz, 1H), 4.81-4.72 (br, 1H), 4.60 (s, 2H), 4.44 (q, J=7.0 Hz, 1H), 3.96 (dd, J=11.0, 3.3 Hz, 1H), 3.91 (dd, J=11.0, 6.0 Hz, 1H), 1.43 (s, 9H), 1.38 (d, J=7.0 Hz, 3H); ¹³C NMR (151 MHz, (CD₃)₂CO) δ 171.4, 166.4, 163.8, 139.2, 134.8, 132.4 (q, J=34.0 Hz), 129.1, 128.7, 128.5, 128.3, 126.1, 124.0 (q, J=271.7 Hz), 85.9, 82.1, 80.2, 73.8, 70.0, 28.1, 16.6; ¹⁹F NMR (471 MHz, CHCl₃) δ −63.0; HRMS (ESI) for C₂₆H₂₉F₆N₂O₇ (M+H)⁺: calculated 595.1873, found 595.1872.

To a CH₂Cl₂ solution (0.5 mL) of compound S16 (7 mg, 11 μmol) was added trifluoroacetic acid (0.5 mL). The reaction mixture was stirred for 1 hour, then concentrated under vacuo and azeotroped with toluene 3 times to remove residual trifluoroacetic acid to afford free carboxylic acid. The reaction mixture was used in the subsequent reaction without further purification. To the CH₂Cl₂ solution (0.5 mL) of carboxylic acid added HATU (6 mg, 15 μmol) and trimethylamine (1.5 mg, 15 μmol). After the mixture was stirred for 30 minutes, aniline (1.5 mg, 15 μmol) was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by preparative thin layer chromatography to afford compound 17. (2 mg, 24% yield for 2 steps). R_{f}=0.4 (40% EtOAc in Hexane); ¹H NMR (500 MHz, (CD₃)₂CO) δ 10.38 (s, 1H), 8.42 (s, 2H), 8.26 (s, 1H), 7.72 (d, J=8.1 Hz, 2H), 7.33-7.21 (m, 7H), 7.05 (t, J=7.1 Hz, 1H), 4.88 (dd, J=5.4, 3.0 Hz, 1H), 4.55-4.47 (m, 3H), 3.99 (dd, J=11.2, 3.0 Hz, 1H), 3.92 (dd, J=11.1, 5.6 Hz, 1H), 1.51 (d, J=7.0 Hz, 3H); ¹³C NMR (151 MHz, (CD₃)₂CO) δ 170.03, 169.96, 169.1, 139.8, 139.0, 134.6, 132.4 (q, J=33.7 Hz), 129.5, 129.1, 128.7, 128.4, 128.3, 126.2, 124.4, 124.0 (q, J=272.1 Hz), 120.2, 86.5, 84.5, 73.9, 70.0, 62.8, 17.4; ¹⁹F NMR (471 MHz, (CD₃)₂CO) δ −63.5; HRMS (ESI) for C₂₈H₂₆F₆N₃O₆ (M+H)⁺: calculated 614.1720, found 614.1720.

Compound 18 was synthesized according to the general procedures described below (Scheme 11).

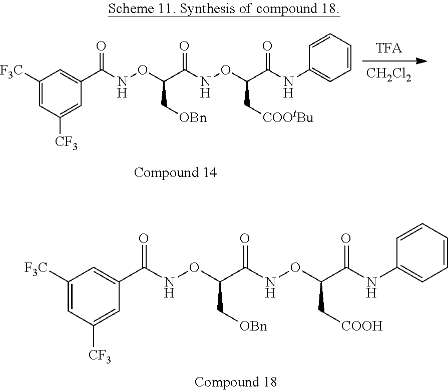

To a CH₂Cl₂ solution (0.5 mL) of compound 14 (13 mg, 18 μmol) was added trifluoroacetic acid (0.5 mL). The reaction mixture was stirred for 1 hour, then concentrated under vacuo and azeotroped with toluene 3 times to remove residual trifluoroacetic acid. The reaction mixture was purified by preparative thin layer chromatography to afford compound 18 (5 mg, 44%). R_{f}=0.3 (0.5% AcOH, 40% EtOAc in Hexane); ¹H NMR (500 MHz, (CD₃)₂CO) δ 10.96 (s, 1H), 8.43 (s, 2H), 8.19 (s, 1H), 7.75 (d, J=7.9 Hz, 2H), 7.35-7.18 (m, 7H), 7.07 (t, J=7.3 Hz, 1H), 4.89 (t, J=4.0 Hz, 1H), 4.84 (dd, J=8.9, 3.8 Hz, 1H), 4.59 (d, J=11.8 Hz, 1H), 4.53 (d, J=11.8 Hz, 1H), 4.03 (dd, J=11.2, 2.9 Hz, 1H), 3.98 (dd, J=11.2, 5.1 Hz, 1H), 3.13 (dd, J=17.2, 3.7 Hz, 1H), 2.85 (dd, J=17.2, 8.9 Hz, 1H); ¹³C NMR (126 MHz, (CD₃)₂CO) δ 173.0, 169.6, 168.4, 162.4, 139.7, 139.0, 135.7, 132.2 (q, J=33.3 Hz), 129.6, 129.1, 128.6, 128.5, 128.3, 125.5, 124.5, 124.2 (q, J=272.2 Hz), 120.1, 86.0, 85.6, 73.9, 70.1, 37.5; ¹⁹F NMR (471 MHz, (CD₃)₂CO) δ −63.4; HRMS (ESI) for C₂₉H₂₆F₆N₃O₈ (M+H)⁺: calculated 658.1619, found 658.1615.

Compound 19 was synthesized according to the general procedures described below (Scheme 12).

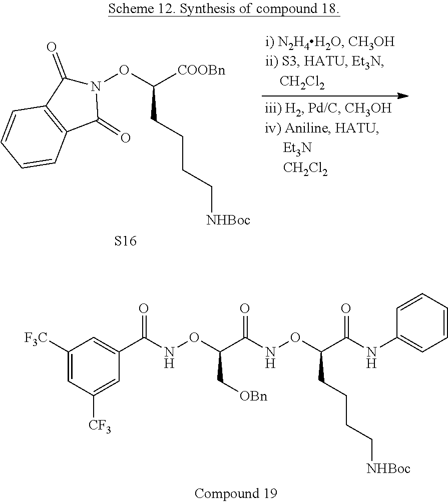

The starting material S15 was prepared according to the procedure described in literature¹³. To starting material S16 (132 mg, 273 μmol) in CH₃OH solution (3 mL) was added N₂H₄·H₂O (41 mg, 818 μmol). After stirring for 1 hour at room temperature, the reaction mixture was concentrated under vacuo. The residue was dissolved in CH₂Cl₂ and filtered. The filtrated was concentrated to afford the free amine as colorless oil, which was used in subsequent reaction without further purification.

To a CH₂Cl₂ solution (3 mL) of S3 (96 mg, 210 μmol) were added HATU (83 mg, 218 μmol) and trimethylamine (22 mg, 218 μmol). After the mixture was stirred for 30 minutes, the free amine was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo, which afforded the benzyl ester dipeptide and used in subsequent reaction without further purification.

To a CH₃OH solution (1 mL) of benzyl ester dipeptide was added Pd/C (3 mg). The reaction mixture was stirred for 1 hour under hydrogen gas, then filtered and concentrated under vacuo to afford free carboxylic acid, which was used in subsequent reaction without further purification. To the CH₂Cl₂ solution (0.25 mL) of carboxylic acid added HATU (12 mg, 31 μmol) and trimethylamine (3 mg, 31 μmol). After the mixture was stirred for 30 minutes, aniline (3 mg, 31 μmol) was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by preparative thin layer chromatography to afford compound 19. (13 mg, 17% yield for 4 steps). R_{f}=0.4 (30% EtOAc in Hexane); ¹H NMR (600 MHz, (CD₃)₂CO) δ 8.43 (s, 2H), 8.18 (s, 1H), 7.74 (dd, J=8.7, 0.9 Hz, 2H), 7.31-7.27 (m, 2H), 7.26-7.20 (m, 5H), 7.05 (tt, J=7.5, 0.9 Hz, 1H), 4.84 (dd, J=5.1, 3.0 Hz, 1H), 4.51 (d, J=11.9 Hz, 1H), 4.47 (d, J=11.9 Hz, 1H), 4.41 (dd, J=8.3, 4.2 Hz, 1H), 3.98 (dd, J=11.1, 3.1 Hz, 1H), 3.93 (dd, J=11.1, 5.3 Hz, 1H), 3.08 (t, J=6.3 Hz, 2H), 2.02-1.93 (m, 1H), 1.88-1.81 (m, 1H), 1.62-1.52 (m, 4H), 1.37 (s, 9H); ¹³C NMR (151 MHz, (CD₃)₂CO) δ 169.9, 169.7, 163.0, 156.7, 139.7, 139.0, 136.3, 132.2 (q, J=33.3 Hz), 129.6, 129.2, 128.6, 128.62, 128.58, 125.4, 124.5, 124.3 (q, J=272.1 Hz), 120.3, 88.1, 85.6, 78.4, 74.1, 70.3, 40.8, 32.3, 28.7, 23.2; ¹⁹F NMR (471 MHz, (CD₃)₂CO) δ −63.4; HRMS (ESI) for C₃₆H₄₁F₆N₄O₈ (M+H)⁺: calculated 771.2823, found 771.2816.

Compound 20 was synthesized according to the general procedures described below (Scheme 13).

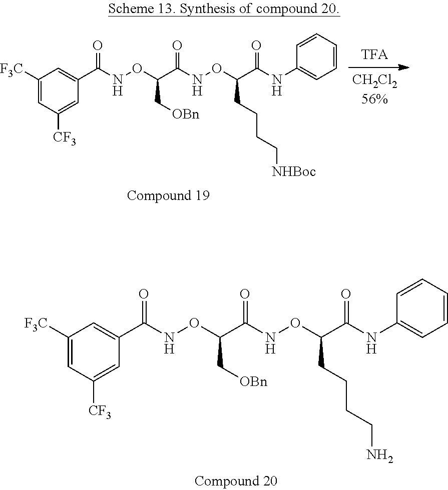 [SMILES: CC(C)(C)OC(=O)NCCCC[C@@H](ONC(=O)[C@@H](COCc1ccccc1)ONC(=O)c1cc(C(F)(F)F)cc(C(F)(F)F)c1)C(=O)Nc1ccccc1 ; NCCCC[C@@H](ONC(=O)[C@@H](COCc1ccccc1)ONC(=O)c1cc(C(F)(F)F)cc(C(F)(F)F)c1)C(=O)Nc1ccccc1]

To a CH₂Cl₂ solution (0.3 mL) of compound 14 (8 mg, 11 μmol) was added trifluoroacetic acid (0.3 mL). The reaction mixture was stirred for 1 hour, then concentrated under vacuo and azeotroped with toluene 3 times to remove residual trifluoroacetic acid. The reaction mixture was purified by preparative thin layer chromatography to afford compound 20 (4 mg, 56%). R_{f}=0.2 (1% NH₄OH, 10% EtOH in CH₂Cl₂); ¹H NMR (600 MHz, CD₃OD) δ 8.27 (s, 2H), 8.09 (s, 1H), 7.59 (dd, J=8.9, 0.8 Hz, 2H), 7.26-7.19 (m, 7H), 7.05 (t, J=7.3 Hz, 1H), 4.74 (t, J=3.7 Hz, 1H), 4.52 (d, J=12.0 Hz, 1H), 4.48 (d, J=12.0 Hz, 1H), 4.47 (dd, J=8.7, 4.4 Hz, 1H), 3.96 (dd, J=11.1, 3.2 Hz, 1H), 3.92 (dd, J=11.1, 4.5 Hz, 1H), 2.96 (t, J=7.5 Hz, 2H), 2.02-1.89 (m, 2H), 1.83-1.63 (m, 2H); ¹³C NMR (151 MHz, CD₃OD) δ 171.1, 169.4, 163.4, 139.00, 138.97, 135.5, 133.0 (q, J=33.7 Hz), 129.7, 129.4, 128.9, 128.7, 128.6, 125.9, 125.6, 124.5 (q, J=272.1 Hz), 121.3, 87.3, 85.1, 74.6, 69.9, 40.5, 32.1, 28.1, 23.1; ¹⁹F NMR (471 MHz, CDCl₃) δ −63.4; HRMS (ESI) for C₃₁H₃₃F₆N₄O₈ (M+H)⁺: calculated 671.2299, found 671.2290.

Compound 21-compound 41 was synthesized according to the general procedures described below (Scheme 14).

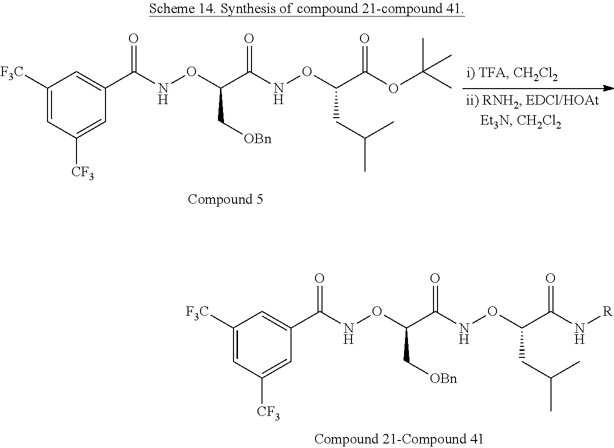

To a CH₂Cl₂ solution (5 mL) of compound 5 (1 mmol, 1 equiv) was added trifluoroacetic acid (5 mL) dropwise at 0° C. The reaction mixture was stirred for 1 hour, then concentrated under vacuo and azeotroped with toluene 3 times to remove residual trifluoroacetic acid to afford free carboxylic acid. The reaction mixture was used in the subsequent reaction without further purification. To the CH₂Cl₂ solution (10 mL) of free carboxylic acid were added HOAt (1.3 mmol, 1.3 equiv), trimethylamine (1.3 mmol, 1 equiv) and EDCI·HCl (1.3 mmol, 1 equiv) subsequently at 0° C. The reaction mixture was stirred for 10 minutes. Free amine (1.3 mmol, 1.3 equiv) was added dropwise at 0° C. The resulting reaction mixture was stirred overnight, then washed sequentially with saturated NaHCO₃ solution and 0.1 M HCl solution. Then the organic layer was dried over anhydrous Na₂SO₄ and concentrated under vacuo. The reaction mixture was purified by flash chromatography to afford compound 21-compound 41.

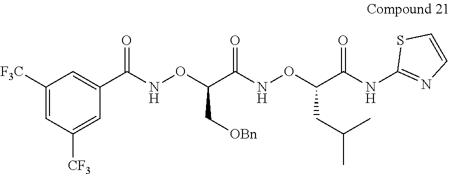 [SMILES: CC(C)C[C@H](ONC(=O)[C@@H](COCc1ccccc1)ONC(=O)c1cc(C(F)(F)F)cc(C(F)(F)F)c1)C(=O)Nc1nccs1]

Compound 21: ¹H NMR (500 MHz, DMSO-d₆) δ 12.56 (s, 1H), 12.21 (s, 1H), 11.78 (s, 1H), 8.34 (s, 2H), 8.29 (s, 1H), 7.42 (d, J=3.4 Hz, 1H), 7.39-7.21 (m, 5H), 7.19 (d, J=2.8 Hz, 1H), 4.78-4.40 (m, 4H), 3.88-3.66 (m, 2H), 1.92-1.78 (m, 1H), 1.67 (ddd, J=14.1, 9.2, 5.0 Hz, 1H), 1.54 (ddd, J=14.0, 8.7, 4.1 Hz, 1H), 0.98 (d, J=6.5 Hz, 3H), 0.91 (d, J=6.6 Hz, 3H); ¹³C NMR (150 MHz, DMSO-d₆) δ 169.5, 165.9, 162.2, 157.2, 137.9, 137.6, 133.8, 130.5 (q, ²J_{C—F}=33.2 Hz), 128.2, 128.0, 127.5, 127.5, 125.3, 123.0 (q, ¹J_{C—F}=273.0 Hz), 113.7, 82.7, 72.3, 72.2, 68.4, 40.3, 24.2, 23.0, 21.7; ¹⁹F NMR (376 MHz, DMSO-d₆) δ −63.4; HRMS (ESI) for C₂₈H₂₉N₄O₆F₆S (M+H)⁺: calculated 663.1707, found 663.1698.

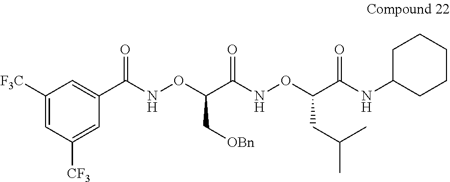 [SMILES: CC(C)C[C@H](ONC(=O)[C@@H](COCc1ccccc1)ONC(=O)c1cc(C(F)(F)F)cc(C(F)(F)F)c1)C(=O)NC1CCCCC1]

Compound 22: ¹H NMR (500 MHz, DMSO-d₆) δ 12.57 (s, 1H), 11.68 (s, 1H), 8.39 (s, 2H), 8.37 (s, 1H), 7.93 (d, J=7.1 Hz, 1H), 7.41-7.21 (m, 5H), 4.67-4.51 (m, 3H), 4.20 (dd, J=9.1, 4.1 Hz, 1H), 3.83-3.72 (m, 2H), 3.49-3.38 (m, 1H), 1.85-1.73 (m, 1H), 1.66-1.40 (m, 7H), 1.24-0.94 (m, 5H), 0.92 (d, J=6.6 Hz, 3H), 0.88 (d, J=6.7 Hz, 3H); ¹³C NMR (126 MHz, DMSO-d₆) δ 169.3, 165.6, 162.1, 137.9, 134.0, 130.6 (q, ²J_{C—F}=33.3 Hz), 128.2, 128.0, 127.55, 127.51, 125.4, 123.0 (q, ¹J_{C—F}=273.1 Hz), 83.7, 82.5, 72.4, 68.5, 47.3, 40.3, 32.2, 32.0, 25.1, 24.43, 24.40, 24.2, 23.0, 21.8; ¹⁹F NMR (376 MHz, DMSO-d₆) δ −61.4; HRMS (ESI) for C₃₁H₃₈N₃O₆F₆ (M+H)⁺: calculated 662.2659, found 662.2658.

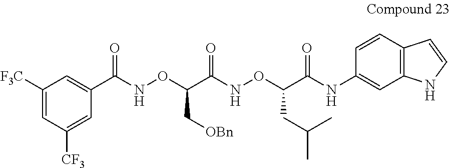 [SMILES: CC(C)C[C@H](ONC(=O)[C@@H](COCc1ccccc1)ONC(=O)c1cc(C(F)(F)F)cc(C(F)(F)F)c1)C(=O)Nc1ccc2cc[nH]c2c1]

Compound 23: ¹H NMR (500 MHz, DMSO-d₆) δ 12.59 (s, 1H), 11.88 (s, 1H), 11.00 (s, 1H), 9.94 (s, 1H), 8.37 (s, 2H), 8.29 (s, 1H), 7.52 (s, 1H), 7.30 (t, J=7.3 Hz, 1H), 7.29-7.22 (m, 6H), 7.21-7.18 (m, 1H), 6.35-6.29 (m, 1H), 4.69-4.61 (m, 1H), 4.55-4.48 (m, 2H), 4.44-4.38 (m, 1H), 3.85-3.75 (m, 2H), 1.69 (ddd, J=14.1, 9.1, 5.0 Hz, 1H), 1.59 (ddd, J=14.1, 8.7, 4.3 Hz, 1H), 1.63-1.54 (m, 1H), 0.98 (d, J=6.6 Hz, 3H), 0.93 (d, J=6.7 Hz, 3H); ¹³C NMR (150 MHz, DMSO-d₆) δ 168.6, 166.0, 162.2, 137.9, 133.9, 132.9, 130.5 (q, ²J_{C—F}=33.3 Hz), 130.3, 128.2, 128.1, 127.6, 127.5, 127.3, 125.9, 125.4, 123.0 (q, ¹J_{C—F}=273.0 Hz), 114.9, 111.03, 111.01, 101.0, 84.3, 82.6, 72.4, 68.5, 40.3, 24.3, 23.1, 21.9; ¹⁹F NMR (376 MHz, DMSO-d₆) δ −63.4; HRMS (ESI) for C₃₃H₃₃N₄O₆F₆ (M+H)⁺: calculated 695.2299, found 695.2297.

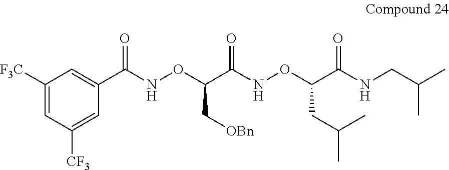 [SMILES: CC(C)CNC(=O)[C@H](CC(C)C)ONC(=O)[C@@H](COCc1ccccc1)ONC(=O)c1cc(C(F)(F)F)cc(C(F)(F)F)c1]

Compound 24: ¹H NMR (500 MHz, DMSO-d₆) δ 12.55 (s, 1H), 11.64 (s, 1H), 8.45-8.32 (m, 3H), 8.05 (s, 1H), 7.37-7.30 (m, 4H), 7.30-7.23 (m, 1H), 4.66-4.50 (m, 3H), 4.26 (dd, J=9.0, 4.3 Hz, 1H), 3.84-3.72 (m, 2H), 2.87-2.66 (m, 2H), 1.86-1.73 (m, 1H), 1.65-1.50 (m, 2H), 1.44 (ddd, J=14.0, 8.7, 4.5 Hz, 1H), 0.93 (d, J=6.6 Hz, 3H), 0.88 (d, J=6.7 Hz, 3H), 0.76 (d, J=4.9 Hz, 3H), 0.74 (d, J=4.8 Hz, 3H); ¹³C NMR (150 MHz, DMSO-d₆) δ 170.3, 165.7, 162.1, 137.9, 134.0, 130.6 (q, ²J_{C—F}=33.3 Hz), 128.2, 128.1, 127.54, 127.52, 125.4, 123.0 (q, ¹J_{C—F}=273.0 Hz), 83.5, 82.4, 72.3, 68.5, 45.7, 40.3, 27.9, 24.2, 23.0, 21.9, 19.9; ¹⁹F NMR (376 MHz, DMSO-d₆) δ −61.4; HRMS (ESI) for C₂₉H₃₆N₃O₆F₆ (M+H)⁺: calculated 636.2503, found 636.2505.

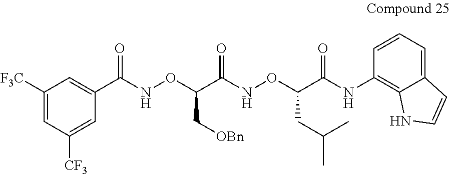 [SMILES: CC(C)C[C@H](ONC(=O)[C@@H](COCc1ccccc1)ONC(=O)c1cc(C(F)(F)F)cc(C(F)(F)F)c1)C(=O)Nc1cccc2cc[nH]c12]

Compound 25: ¹H NMR (500 MHz, DMSO-d₆) δ 12.59 (s, 1H), 11.94 (s, 1H), 10.63 (s, 1H), 9.89 (s, 1H), 8.34 (s, 2H), 8.29 (s, 1H), 7.35-7.20 (m, 8H), 6.86 (t, J=7.5 Hz, 1H), 6.42 (t, J=2.3 Hz, 1H), 4.71-4.61 (m, 1H), 4.59-4.46 (m, 3H), 3.80 (d, J=4.1 Hz, 2H), 1.99-1.87 (m, 1H), 1.80 (ddd, J=14.4, 9.4, 5.0 Hz, 1H), 1.62 (ddd, J=14.0, 8.9, 4.2 Hz, 1H), 1.02 (d, J=6.5 Hz, 3H), 0.96 (d, J=6.6 Hz, 3H); ¹³C NMR (126 MHz, DMSO-d₆) δ 169.4, 165.9, 162.2, 137.8, 133.8, 130.5 (q, ²J_{C—F}=33.2 Hz), 129.1, 128.3, 128.2, 128.0, 127.54, 127.48, 125.3, 125.1, 123.0 (q, ¹J_{C—F}=273.0 Hz), 122.4, 118.7, 116.9, 114.0, 101.6, 84.1, 82.6, 72.4, 68.4, 40.3, 24.3, 23.1, 21.8; ¹⁹F NMR (376 MHz, DMSO-d₆) δ −61.4; HRMS (ESI) for C₃₃H₃₃N₄O₆F₆ (M+H)⁺: calculated 695.2299, found 695.2293.

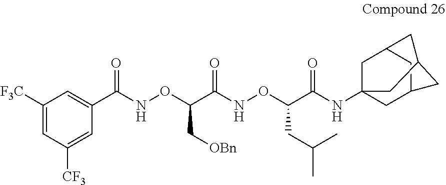 [SMILES: CC(C)C[C@H](ONC(=O)[C@@H](COCc1ccccc1)ONC(=O)c1cc(C(F)(F)F)cc(C(F)(F)F)c1)C(=O)NC12CC3CC(CC(C3)C1)C2]

Compound 26: ¹H NMR (320 K, 500 MHz, DMSO-d₆) δ 12.48 (s, 1H), 11.60 (s, 1H), 8.38 (s, 2H), 8.31 (s, 1H), 7.46 (s, 1H), 7.36-7.30 (m, 4H), 7.31-7.26 (m, 1H), 4.66-4.55 (m, 3H), 4.10 (dd, J=8.7, 4.4 Hz, 1H), 3.86-3.77 (m, 2H), 1.97-1.90 (m, 3H), 1.90-1.82 (m, 6H), 1.81-1.75 (m, 1H), 1.63-1.43 (m, 8H), 0.92 (d, J=6.6 Hz, 3H), 0.89 (d, J=6.7 Hz, 3H); ¹³C NMR (150 MHz, DMSO-d₆) δ 169.5, 165.7, 162.3, 137.9, 134.0, 130.5 (q, ²J_{C—F}=33.3 Hz), 128.2, 128.1, 127.57, 127.55, 125.5, 123.0 (q, ¹J_{C—F}=273.0 Hz), 84.1, 82.3, 72.4, 68.5, 50.6, 40.6, 40.3, 35.9, 28.7, 24.2, 23.1, 21.8; ¹⁹F NMR (376 MHz, DMSO-d₆) δ −61.4; HRMS (ESI) for C₃₅H₄₂N₃O₆F₆ (M+H)⁺: calculated 714.2972, found 714.2968.

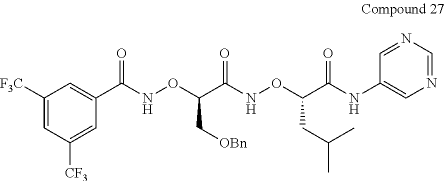 [SMILES: CC(C)C[C@H](ONC(=O)[C@@H](COCc1ccccc1)ONC(=O)c1cc(C(F)(F)F)cc(C(F)(F)F)c1)C(=O)Nc1cncnc1]

Compound 27: ¹H NMR (500 MHz, DMSO-d₆) δ 12.52 (s, 1H), 11.84 (s, 1H), 10.44 (s, 1H), 8.88 (s, 2H), 8.83 (s, 1H), 8.31 (s, 2H), 8.26 (s, 1H), 7.56-7.44 (m, 5H), 4.64-4.57 (m, 1H), 4.57-4.42 (m, 3H), 3.85-3.71 (m, 2H), 1.95-1.85 (m, 1H), 1.74-1.65 (ddd, J=14.4, 9.6, 4.9 Hz, 1H), 1.56-1.49 (ddd, J=14.3, 8.8, 4.0 Hz, 1H), 1.00 (d, J=6.6 Hz, 3H), 0.93 (d, J=6.7 Hz, 3H); ¹³C NMR (150 MHz, DMSO-d₆) δ 170.5, 165.6, 162.2, 153.2, 147.1, 137.8, (133.8), 133.7, 130.4 (q, ²J_{C—F}=33.3 Hz), 128.2, 128.0, 127.49, 127.46, 125.3, 122.9 (q, ¹J_{C—F}=273.0 Hz), 83.6, 82.3, 72.3, 68.2, 40.3, 24.2, 23.0, 21.7; ¹⁹F NMR (376 MHz, DMSO-d₆) δ −61.4; HRMS (ESI) for C₂₉H₃₀N₅O₆F₆ (M+H)⁺: calculated 658.2091, found 658.2095.

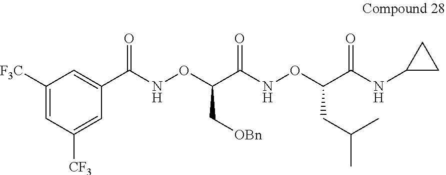 [SMILES: CC(C)C[C@H](ONC(=O)[C@@H](COCc1ccccc1)ONC(=O)c1cc(C(F)(F)F)cc(C(F)(F)F)c1)C(=O)NC1CC1]

Compound 28: ¹H NMR (500 MHz, DMSO-d₆) δ 12.56 (s, 1H), 11.66 (s, 1H), 8.39 (s, 2H), 8.36 (s, 1H), 8.11 (d, J=2.9 Hz, 1H), 7.39-7.30 (m, 5H), 7.30-7.24 (m, 1H), 4.65-4.52 (m, 3H), 4.18 (dd, J=9.1, 4.2 Hz, 1H), 3.80-3.74 (m, 2H), 1.83-1.73 (m, 1H), 1.52 (ddd, J=14.2, 9.1, 5.2 Hz, 1H), 1.42 (ddd, J=14.1, 8.8, 4.4 Hz, 1H), 0.91 (d, J=6.6 Hz, 3H), 0.87 (d, J=6.7 Hz, 3H), 0.56-0.49 (m, 2H), 0.39-0.29 (m, 2H); ¹³C NMR (150 MHz, DMSO-d₆) δ 171.6, 165.8, 162.2, 137.9, 134.1, 130.5 (q, ²J_{C—F}=33.4 Hz), 128.2, 128.1, 127.6, 127.5, 125.3, 123.0 (q, ¹J_{C—F}=273.0 Hz), 83.6, 82.4, 72.3, 68.5, 40.3, 24.1, 23.0, 22.0, 21.8, 5.6, 5.4; ¹⁹F NMR (376 MHz, DMSO-d₆) δ −61.4; HRMS (ESI) for C₂₈H₃₁N₃O₆F₆ (M+H)⁺: calculated 620.2190, found 620.2192.

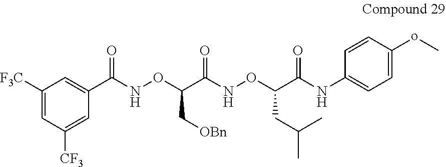

Compound 29: ¹H NMR (500 MHz, DMSO-d₆) δ 12.55 (s, 1H), 11.85 (s, 1H), 9.95 (s, 1H), 8.35 (s, 2H), 8.31 (s, 1H), 7.44 (d, J=8.5 Hz, 2H), 7.34-7.21 (m, 5H), 6.80 (d, J=8.8 Hz, 2H), 4.68-4.60 (m, 1H), 4.56-4.49 (m, 2H), 4.43-4.35 (m, 1H), 3.85-3.74 (m, 2H), 3.69 (s, 3H), 1.93-1.80 (m, 1H), 1.66 (ddd, J=14.1, 9.2, 5.1 Hz, 1H), 1.53 (ddd, J=14.0, 8.7, 4.0 Hz, 1H), 0.97 (d, J=6.6 Hz, 3H), 0.92 (d, J=6.7 Hz, 3H); ¹³C NMR (150 MHz, DMSO-d₆) δ 168.8, 166.0, 162.1, 155.4, 137.9, 133.9, 131.5, 130.5 (q, ²J_{C—F}=33.3 Hz), 128.2, 128.1, 127.6, 127.5, 125.3, 123.0 (q, ¹J_{C—F}=273.1 Hz), 120.9, 113.7, 84.0, 82.4, 72.4, 68.4, 55.1, 40.3, 24.2, 23.1, 21.8; ¹⁹F NMR (376 MHz, DMSO-d₆) δ −61.4; HRMS (ESI) for C₃₂H₃₄N₃O₇F₆ (M+H)⁺: calculated 686.2295, found 686.2290.

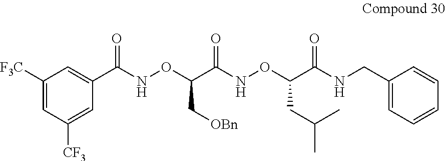 [SMILES: CC(C)C[C@H](ONC(=O)[C@@H](COCc1ccccc1)ONC(=O)c1cc(C(F)(F)F)cc(C(F)(F)F)c1)C(=O)NCc1ccccc1]

Compound 30: ¹H NMR (500 MHz, DMSO-d₆) δ 12.60 (s, 1H), 11.70 (s, 1H), 8.61 (s, 1H), 8.39 (s, 2H), 8.33 (s, 1H), 7.37-7.22 (m, 7H), 7.22-7.15 (m, 3H), 4.67-4.59 (m, 1H), 4.59-4.50 (m, 2H), 4.34 (dd, J=8.9, 4.1 Hz, 1H), 4.25-4.15 (m, 2H), 3.80-3.71 (m, 2H), 1.87-1.76 (m, 1H), 1.59 (ddd, J=14.1, 8.9, 5.3 Hz, 1H), 1.50 (ddd, J=14.1, 8.5, 4.3 Hz, 1H), 0.93 (d, J=6.6 Hz, 3H), 0.88 (d, J=6.6 Hz, 3H); ¹³C NMR (150 MHz, DMSO-d₆) δ 170.5, 165.8, 162.2, 138.9, 137.9, 134.0, 130.5 (q, ²J_{C—F}=33.3 Hz), 128.21, 128.20, 128.1, 127.53, 127.50, 127.0, 126.7, 125.4, 123.0 (q, ¹J_{C—F}=273.0 Hz), 84.5, 82.7, 72.3, 68.6, 41.8, 40.3, 24.1, 23.0, 21.8; ¹⁹F NMR (376 MHz, DMSO-d₆) δ −61.4; HRMS (ESI) for C₃₂H₃₄N₃O₆F₆ (M+H)⁺: calculated 670.2346, found 670.2342.

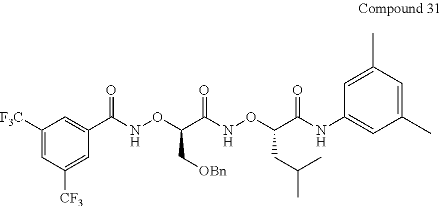 [SMILES: Cc1cc(C)cc(NC(=O)[C@H](CC(C)C)ONC(=O)[C@@H](COCc2ccccc2)ONC(=O)c2cc(C(F)(F)F)cc(C(F)(F)F)c2)c1]

Compound 31: ¹H NMR (320K, 500 MHz, DMSO-d₆) δ 12.47 (s, 1H), 11.75 (s, 1H), 9.87 (s, 1H), 8.34 (s, 2H), 8.26 (s, 1H), 7.33-7.23 (m, 5H), 7.19 (s, 2H), 6.68 (s, 1H), 4.71-4.59 (m, 1H), 4.57-4.48 (m, 2H), 4.40 (dd, J=8.9, 4.4 Hz, 1H), 3.86-3.77 (m, 2H), 2.22 (s, 6H), 1.95-1.80 (m, 1H), 1.67 (ddd, J=14.3, 8.9, 5.4 Hz, 1H), 1.58 (ddd, J=14.1, 8.4, 4.5 Hz, 1H), 0.97 (d, J=6.5 Hz, 3H), 0.93 (d, J=6.7 Hz, 3H); ¹³C NMR (150 MHz, DMSO-d₆) δ 169.1, 165.9, 162.2, 138.2, 137.9, 137.7, 133.8, 130.4 (q, ²J_{C—F}=33.3 Hz), 128.2, 128.1, 127.52, 127.47, 125.2, 125.0, 123.0 (q, ¹J_{C—F}=273.0 Hz), 117.1, 84.0, 82.6, 72.3, 68.4, 40.3, 24.2, 23.0, 21.8, 21.0; ¹⁹F NMR (376 MHz, DMSO-d₆) δ −61.4; HRMS (ESI) for C₃₃H₃₆N₃O₆F₆ (M+H)⁺: calculated 684.2503, found 684.2500.

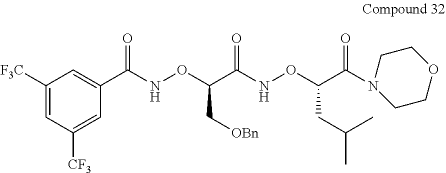 [SMILES: CC(C)C[C@H](ONC(=O)[C@@H](COCc1ccccc1)ONC(=O)c1cc(C(F)(F)F)cc(C(F)(F)F)c1)C(=O)N1CCOCC1]

Compound 32: ¹H NMR (500 MHz, DMSO-d₆) δ 12.58 (s, 1H), 11.47 (s, 1H), 8.41 (s, 2H), 8.38 (s, 1H), 7.42-7.32 (m, 4H), 7.32-7.25 (m, 1H), 4.81-4.71 (m, 1H), 4.70-4.62 (m, 1H), 4.62-4.51 (m, 2H), 3.86-3.66 (m, 2H), 3.49-3.39 (m, 3H), 3.32-3.26 (m, 4H), 3.23-3.13 (m, 1H), 1.85-1.72 (m, 1H), 1.60-1.50 (m, 1H), 1.45-1.36 (m, 1H), 0.93 (d, J=6.6 Hz, 3H), 0.86 (d, J=6.7 Hz, 3H); ¹³C NMR (150 MHz, DMSO-d₆) δ 168.0, 167.2, 162.2, 138.0, 134.1, 130.6 (q, ²J_{C—F}=33.4 Hz), 128.2, 128.1, 127.6, 127.5, 125.5, 123.0 (q, ¹J_{C—F}=273.0 Hz), 82.0, 79.3, 72.2, 68.5, 65.9, 45.2, 40.3, 24.2, 22.8, 21.9; ¹⁹F NMR (376 MHz, DMSO-d₆) δ −61.4; HRMS (ESI) for C₂₉H₃₄N₃O₇F₆ (M+H)⁺: calculated 650.2295, found 650.2294.

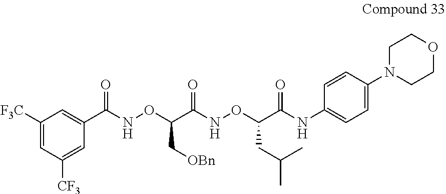 [SMILES: CC(C)C[C@H](ONC(=O)[C@@H](COCc1ccccc1)ONC(=O)c1cc(C(F)(F)F)cc(C(F)(F)F)c1)C(=O)Nc1ccc(N2CCOCC2)cc1]

Compound 33: ¹H NMR (500 MHz, DMSO-d₆) δ 12.54 (s, 1H), 11.84 (s, 1H), 9.91 (s, 1H), 8.35 (s, 2H), 8.38 (s, 1H), 7.40 (d, J=8.8 Hz, 2H), 7.33-7.23 (m, 5H), 6.82 (d, J=9.0 Hz, 2H), 4.68-4.58 (m, 1H), 4.56-4.48 (m, 2H), 4.38 (dd, J=9.2, 4.0 Hz, 1H), 3.83-3.75 (m, 2H), 3.75-3.67 (m, 4H), 3.06-2.96 (m, 4H), 1.91-1.80 (m, 1H), 1.66 (ddd, J=14.2, 9.2, 5.2 Hz, 1H), 1.54 (ddd, J=14.0, 8.8, 4.3 Hz, 1H), 0.97 (d, J=6.5 Hz, 3H), 0.91 (d, J=6.6 Hz, 3H); ¹³C NMR (126 MHz, DMSO-d₆) δ 168.6, 166.0, 162.2, 147.5, 137.9, 133.9, 130.5 (q, ²J_{C—F}=32.6 Hz) (130.6), 128.2, 128.0, 127.6, 127.5, 125.31, 123.0 (q, ¹J_{C—F}=272.9 Hz), 120.4, 115.3, 84.1, 82.5, 72.4, 68.4, 66.1, 48.9, 40.3, 24.2, 23.0, 21.8; ¹⁹F NMR (376 MHz, DMSO-d₆) δ −61.3; HRMS (ESI) for C₃₅H₃₉N₄O₇F₆ (M+H)⁺: calculated 741.2717, found 741.2710.

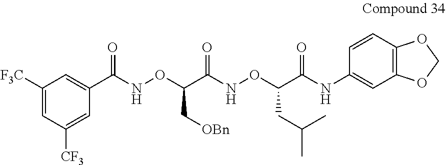 [SMILES: CC(C)C[C@H](ONC(=O)[C@@H](COCc1ccccc1)ONC(=O)c1cc(C(F)(F)F)cc(C(F)(F)F)c1)C(=O)Nc1ccc2c(c1)OCO2]

Compound 34: ¹H NMR (500 MHz, DMSO-d₆) δ 12.54 (s, 1H), 11.85 (s, 1H), 9.97 (s, 1H), 8.34 (s, 2H), 8.30 (s, 1H), 7.39-7.29 (m, 5H), 7.18 (s, 1H), 6.92 (d, J=7.8 Hz, 1H), 6.76 (d, J=8.6 Hz, 1H), 5.95 (s, 2H), 4.68-4.58 (br, 1H), 4.57-4.49 (m, 2H), 4.18 (dd, J=8.9, 3.5 Hz, 1H), 3.83-3.74 (m, 2H), 1.92-1.80 (m, 1H), 1.65 (ddd, J=14.2, 9.3, 5.1 Hz, 1H), 1.51 (ddd, J=14.1, 8.8, 4.1 Hz, 1H), 0.97 (d, J=6.6 Hz, 3H), 0.91 (d, J=6.7 Hz, 3H); ¹³C NMR (150 MHz, DMSO-d₆) δ 168.9, 165.9, 162.1, 146.9, 143.1, 137.9, 133.9, 132.7, 130.4 (q, ²J_{C—F}=33.3 Hz), 128.2, 128.0, 127.6, 127.5, 125.3, 123.0 (q, ¹J_{C—F}=273.0 Hz), 112.2, 107.8, 101.4, 100.9, 83.9, 82.4, 72.3, 68.4, 40.3, 24.2, 23.2, 21.8; ¹⁹F NMR (376 MHz, DMSO-d₆) δ −61.41; HRMS (ESI) for C₃₂H₃₂N₃O₈F₆ (M+H)⁺: calculated 700.2088, found 700.2081.

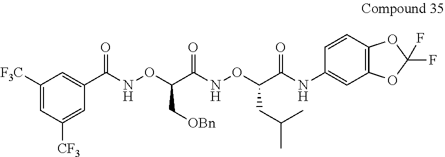 [SMILES: CC(C)C[C@H](ONC(=O)[C@@H](COCc1ccccc1)ONC(=O)c1cc(C(F)(F)F)cc(C(F)(F)F)c1)C(=O)Nc1ccc2c(c1)OC(F)(F)O2]

Compound 35: ¹H NMR (500 MHz, CD₃OD) δ 8.26 (s, 2H), 8.08 (s, 1H), 7.60 (d, J=1.1 Hz, 1H), 7.31-7.13 (m, 6H), 6.99 (d, J=8.7 Hz, 1H), 4.74-4.67 (m, 1H), 4.58-4.41 (m, 3H), 3.98-3.86 (m, 2H), 1.99-1.90 (m, 1H), 1.79 (ddd, J=14.4, 9.5, 5.0 Hz, 1H), 1.66 (ddd, J=14.3, 8.9, 3.8 Hz, 1H), 1.02 (d, J=6.6 Hz, 3H), 0.97 (d, J=6.7 Hz, 3H); ¹³C NMR (150 MHz, CDCl₃) δ 170.2, 167.1, 165.4, 143.9, 140.4, 137.1, 134.0, 132.8 (q, ²J_{C—F}=34.6 Hz), 132.4, 131.9 (t, ¹J_{C—F}=254.9 Hz), 128.9, 128.6, 128.1, 127.9, 126.6, 122.8 (q, ¹J_{C—F}=273.0 Hz), 115.1, 109.4, 103.1, 86.4, 86.0, 74.2, 69.2, 40.7, 25.0, 23.4, 21.3; ¹⁹F NMR (376 MHz, DMSO-d₆) δ −49.2 (2F), −61.5 (6F); HRMS (ESI) for C₃₂H₃₀N₃O₈F₈ (M+H)⁺: calculated 736.1900, found 736.1894.

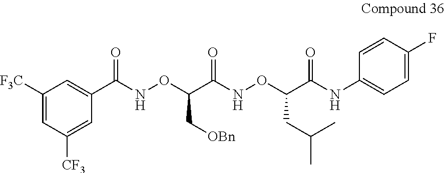 [SMILES: CC(C)C[C@H](ONC(=O)[C@@H](COCc1ccccc1)ONC(=O)c1cc(C(F)(F)F)cc(C(F)(F)F)c1)C(=O)Nc1ccc(F)cc1]

Compound 36: ¹H NMR (500 MHz, DMSO-d₆) δ 12.55 (s, 1H), 11.87 (s, 1H), 10.14 (s, 1H), 8.34 (s, 2H), 8.30 (s, 1H), 7.62-7.48 (m, 2H), 7.37-7.18 (m, 5H), 7.05 (t, J=8.7 Hz, 2H), 4.68-4.59 (m, 1H), 4.56-4.48 (m, 2H), 4.47-4.40 (m, 1H), 3.80-3.70 (m, 2H), 1.92-1.81 (m, 1H), 1.68 (ddd, J=14.2, 9.1, 5.2 Hz, 1H), 1.54 (ddd, J=14.1, 8.6, 4.0 Hz, 1H), 0.97 (d, J=6.6 Hz, 3H), 0.91 (d, J=6.7 Hz, 3H); ¹³C NMR (126 MHz, DMSO-d₆) δ 169.3, 165.9, 162.3, 158.1 (d, ¹J_{C—F}=240.1 Hz), 137.9, 134.8, 133.9, 130.5 (q, ²J_{C—F}=33.3 Hz), 128.2, 128.1, 127.6, 127.5, 125.3, 123.0 (q, ¹J_{C—F}=273.0 Hz), 121.1 (d, ³J_{C—F}=7.4 Hz), 115.1 (d, 2J_{C—F}=22.2 Hz), 84.0, 82.4, 72.4, 68.4, 40.3, 24.3, 23.1, 21.8; ¹⁹F NMR (376 MHz, DMSO-d₆) δ −61.4 (6F), −119.1 (1F); HRMS (ESI) for C₃₁H₃₁N₃O₆F₇ (M+H)⁺: calculated 674.2096, found 674.2091.

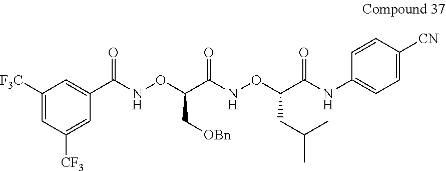 [SMILES: CC(C)C[C@H](ONC(=O)[C@@H](COCc1ccccc1)ONC(=O)c1cc(C(F)(F)F)cc(C(F)(F)F)c1)C(=O)Nc1ccc(C#N)cc1]

Compound 37: ¹H NMR (500 MHz, DMSO-d₆) δ 12.54 (s, 1H), 11.90 (s, 1H), 10.46 (s, 1H), 8.30 (s, 2H), 8.29 (s, 1H), 7.75-7.56 (m, 4H), 7.38-7.21 (m, 5H), 4.64-4.56 (m, 1H), 4.55-4.43 (m, 3H), 3.84-3.72 (m, 2H), 1.92-1.82 (m, 1H), 1.68 (ddd, J=14.6, 9.2, 5.3 Hz, 1H), 1.51 (ddd, J=14.0, 9.0, 4.5 Hz, 1H), 0.98 (d, J=6.6 Hz, 3H), 0.92 (d, J=6.7 Hz, 3H); ¹³C NMR (126 MHz, CDCl₃) δ 170.4, 167.2, 165.3, 142.2, 137.1, 133.3, 132.7 (q, ²J_{C—F}=33.3 Hz), 128.8, 128.5, 128.1, 127.9, 127.7, 126.4, 122.1 (q, ¹J_{C—F}=273.0 Hz), 120.0, 119.2, 106.9, 86.5, 86.0, 74.2, 69.1, 40.8, 24.9, 23.4, 21.6; ¹⁹F NMR (376 MHz, DMSO-d₆) δ −61.37; HRMS (ESI) for C₃₂H₃₁N₄O₆F₆ (M+H)⁺: calculated 681.2142, found 681.2139.

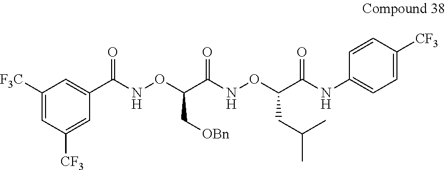 [SMILES: CC(C)C[C@H](ONC(=O)[C@@H](COCc1ccccc1)ONC(=O)c1cc(C(F)(F)F)cc(C(F)(F)F)c1)C(=O)Nc1ccc(C(F)(F)F)cc1]

Compound 38: ¹H NMR (500 MHz, DMSO-d₆) δ 12.53 (s, 1H), 11.89 (s, 1H), 10.37 (s, 1H), 8.31 (s, 2H), 8.22 (s, 1H), 7.69 (d, J=7.8 Hz, 2H), 7.55 (d, J=8.3 Hz, 2H), 7.36-7.20 (m, 5H), 4.64-4.57 (m, 1H), 4.56-4.46 (m, 3H), 3.82-3.73 (m, 2H), 1.92-1.83 (m, 1H), 1.68 (ddd, J=14.4, 9.5, 4.8 Hz, 1H), 1.53 (ddd, J=14.1, 8.9, 3.8 Hz, 1H), 0.99 (d, J=6.6 Hz, 3H), 0.92 (d, J=6.7 Hz, 3H); ¹³C NMR (150 MHz, DMSO-d₆) δ 170.1, 165.9, 162.2, 141.9, 137.9, 134.1, 130.4 (q, ²J_{C—F}=32.9 Hz), 128.2, 128.0, 127.53, 127.49, 125.8 (q, ³J_{C—F}=3.3 Hz), 125.1, 124.3 (q, ¹J_{C—F}=271.3 Hz), 123.5 (q, ²J_{C—F}=31.9 Hz), 123.0 (q, ¹J_{C—F}=273.2 Hz), 119.2, 83.8, 82.4, 72.4, 68.4, 40.3, 24.3, 24.0, 21.7; ¹⁹F NMR (376 MHz, DMSO-d₆) δ −60.6 (3F), −61.4 (6F); HRMS (ESI) for C₃₂H₃₁N₃O₆F₉ (M+H)⁺: calculated 724.2063, found 724.2064.

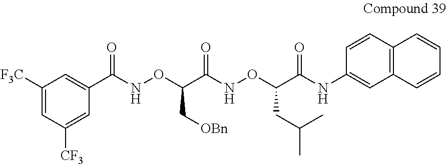 [SMILES: CC(C)C[C@H](ONC(=O)[C@@H](COCc1ccccc1)ONC(=O)c1cc(C(F)(F)F)cc(C(F)(F)F)c1)C(=O)Nc1ccc2ccccc2c1]

Compound 39: ¹H NMR (500 MHz, DMSO-d₆) δ 12.57 (s, 1H), 11.92 (s, 1H), 10.29 (s, 1H), 8.34 (s, 2H), 8.24 (s, 1H), 8.22 (s, 1H), 7.80 (t, J=9.4 Hz, 2H), 7.73 (d, J=8.0 Hz, 1H), 7.55 (d, J=8.4 Hz, 1H) 7.46 (t, J=7.1 Hz, 1H), 7.40 (t, J=7.0 Hz, 1H), 7.32-7.20 (m, 5H), 4.72-4.59 (m, 1H), 4.55-4.45 (m, 3H), 3.86-3.74 (m, 2H), 1.96-1.84 (m, 1H), 1.73 (ddd, J=14.4, 9.0, 5.6 Hz, 1H), 1.59 (ddd, J=14.1, 8.3, 4.2 Hz, 1H), 0.99 (d, J=6.6 Hz, 3H), 0.94 (d, J=6.7 Hz, 3H); ¹³C NMR (150 MHz, DMSO-d₆) δ 169.6, 166.0, 162.3, 137.8, 135.9, 134.0, 133.2, 130.4 (q, ²J_{C—F}=33.3 Hz), 129.9, 128.21, 128.17, 128.0, 127.54, 127.47, 127.43, 127.2, 126.4, 125.2, 124.7, 123.0 (q, ¹J_{C—F}=273.1 Hz), 120.0, 115.6, 84.1, 82.5, 72.4, 68.4, 40.3, 24.3, 23.1, 21.8; ¹⁹F NMR (376 MHz, DMSO-d₆) δ −61.4; HRMS (ESI) for C₃₅H₃₄N₃O₆F₆ (M+H)⁺: calculated 706.2346, found 706.2345.

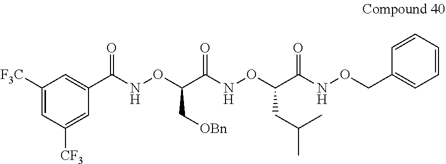 [SMILES: CC(C)C[C@H](ONC(=O)[C@@H](COCc1ccccc1)ONC(=O)c1cc(C(F)(F)F)cc(C(F)(F)F)c1)C(=O)NOCc1ccccc1]

Compound 40: ¹H NMR (500 MHz, DMSO-d₆) δ 12.57 (s, 1H), 11.66 (s, 1H), 11.33 (s, 1H), 8.41 (s, 2H), 8.33 (s, 1H), 7.41-7.22 (m, 10H), 4.79-4.65 (m, 2H), 4.65-4.60 (m, 1H), 4.60-4.52 (m, 2H), 4.27-4.18 (m, 1H), 3.85-3.72 (m, 2H), 1.76-1.65 (m, 1H), 1.61-1.51 (m, 1H), 1.44-1.36 (m, 1H), 0.90 (d, J=6.5 Hz, 3H), 0.84 (d, J=6.6 Hz, 3H); ¹³C NMR (150 MHz, DMSO-d₆) δ 167.2, 165.8, 162.3, 137.9, 135.6, 134.0, 130.5 (q, ²J_{C—F}=33.4 Hz), 128.8, 128.3, 128.24 (128.22), 127.6, 127.53 (127.52), 125.5, 123.0 (q, ¹J_{C—F}=273.0 Hz), 82.6, 81.6, 76.9, 72.3, 68.6, 40.3, 24.0, 22.8, 22.0; ¹⁹F NMR (376 MHz, DMSO-d₆) δ −61.4; (M+H)⁺; HRMS (ESI) for C₃₂H₃₃N₃O₇F₆ (M+H)⁺: calculated 686.2292, found 686.2295.

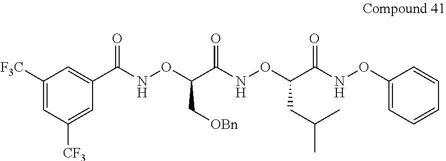 [SMILES: CC(C)C[C@H](ONC(=O)[C@@H](COCc1ccccc1)ONC(=O)c1cc(C(F)(F)F)cc(C(F)(F)F)c1)C(=O)NOc1ccccc1]

Compound 41: ¹H NMR (500 MHz, DMSO-d₆) δ 12.62 (s, 1H), 12.10 (s, 1H), 11.81 (s, 1H), 8.40 (s, 2H), 8.33 (s, 1H), 7.38-7.31 (m, 4H), 7.32-7.26 (m, 1H), 7.23 (t, J=7.4 Hz, 2H), 7.04-6.90 (m, 3H), 4.72-4.66 (m, 1H), 4.62-4.55 (m, 2H), 4.48-4.50 (m, 1H), 3.86-3.77 (m, 2H), 1.91-1.80 (m, 1H), 1.75-1.66 (m, 1H), 1.58-1.47 (m, 1H), 0.98 (d, J=6.5 Hz, 3H), 0.93 (d, J=6.6 Hz, 3H); ¹³C NMR (150 MHz, DMSO-d₆) δ 168.0, 166.1, 162.4, 159.1, 137.9, 133.8, 130.5 (q, ²J_{C—F}=33.3 Hz), 129.4, 128.22, (128.21), 127.6, 127.5, 125.5, 123.0 (q, ¹J_{C—F}=273.0 Hz), 122.3, 112.7, 82.9, 81.5, 72.3, 68.6, 40.3, 24.1, 22.9, 21.9; ¹⁹F NMR (376 MHz, DMSO-d₆) δ −61.4; HRMS (ESI) for C₃₁H₃₂N₃O₇F₆ (M+H)⁺: calculated 672.2139, found 672.2139.

Compound 42-compound 52 were synthesized according to the general procedures described below (Scheme 15).

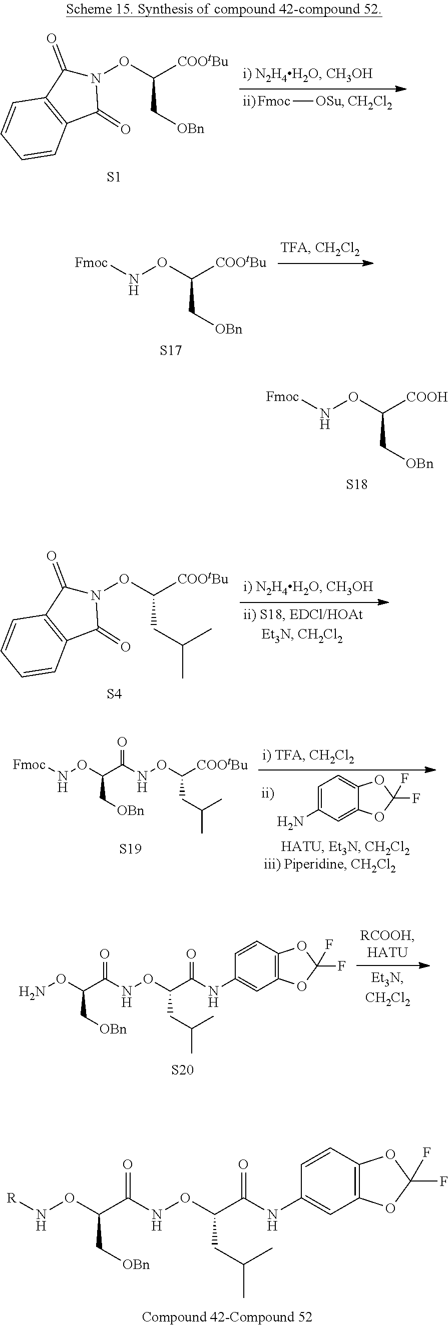

S2 was synthesized from S1 (2 g, 5.03 mmol) according to the Scheme 1 without further purification. To S2 in anhydrous CH₂Cl₂ (40 mL), Fmoc-OSu (1.767 g, 5.24 mmol) was added. The reaction was stirred overnight and then concentrated under vacuo. The reaction mixture was purified by flash chromatography to afford white solid S17 (1.858 g, 90% yield for 2 steps). ¹H NMR (300 MHz, CDCl₃) δ 8.28 (s, 1H), 7.69 (d, J=7.4 Hz, 2H), 7.55 (d, J=7.4 Hz, 2H), 7.40-7.12 (m, 9H), 4.68-4.33 (m, 5H), 4.18 (t, J=6.7 Hz, 1H), 3.89-3.70 (m, 2H), 1.46 (s, 9H); ¹³C NMR (76 MHz, CDCl₃) δ 168.3, 156.9, 137.6, 128.3, 127.8, 127.7, 127.7, 127.1, 125.0, 119.9, 84.0, 82.6, 73.5, 68.7, 67.4, 46.9, 28.0; HRMS (EI, 20 eV) for C₂₉H₃₁NO₆ (M+H)⁺: calculated 489.2146, found 489.2135.

To a CH₂Cl₂ solution (13 mL) of compound S17 (1.85 g, 3.906 mmol) was added trifluoroacetic acid (13 mL) dropwise at 0° C. The reaction mixture was stirred for 1 hour, then concentrated under vacuo and azeotroped with toluene 3 times to remove residual trifluoroacetic acid to afford compound S18, which was used in subsequent reaction without further purification.

To starting material S4 (1.45 g, 4.36 mmol) in CH₃OH solution (30 mL) was added N₂H₄·H₂O (657 mg, 13 mmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To a CH₂Cl₂ solution (3 mL) of S18 (1.34 g, 1.341 mmol) were added HOAt (638 mg, 4.69 mmol), trimethylamine (469 mg, 4.69 mmol) and EDCI·HCl (638 mg, 4.69 mmol) subsequently at 0° C. After the mixture was stirred for 10 minutes, the free amine was added dropwise at 0° C. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford compounds S19 (1.66 g, 69% yield for 3 steps). R_{f}=0.5 (30% EtOAc in Hexane); ¹H NMR (300 MHz, CDCl₃) δ 10.95 (s, 1H), 8.47 (s, 1H), 7.71 (d, J=7.4 Hz, 2H), 7.50 (d, J=5.7 Hz, 2H), 7.36 (d, J=7.3 Hz, 2H), 7.32-7.15 (m, 7H), 4.59-4.34 (m, 5H), 4.30 (dd, J=6.9, 2.3 Hz, 1H), 4.16 (t, J=6.4 Hz, 1H), 3.77 (dd, J=11.0, 2.6 Hz, 1H), 3.58 (dd, J=11.0, 7.4 Hz, 1H), 2.07-1.88 (m, 1H), 1.75 (ddd, J=13.9, 8.4, 5.6 Hz, 1H), 1.55 (ddd, J=13.5, 8.6, 4.3 Hz, 1H), 1.46 (s, 9H), 1.01 (d, J=6.5 Hz, 3H), 0.95 (d, J=6.6 Hz, 3H); ¹³C NMR (76 MHz, CDCl₃) δ 171.0, 165.9, 158.5, 143.18, 143.16, 141.27, 141.25, 137.3, 128.4, 127.9, 127.8, 127.2, 124.9, 124.8, 120.0, 86.2, 82.9, 82.1, 73.4, 69.2, 67.9, 46.8, 39.9, 27.9, 24.7, 24.0, 21.8; HRMS (ESI) for C₃₅H₄₃N₂O₈ (M+H)⁺: calculated 619.3014, found 619.3007.

To a CH₂Cl₂ solution (15 mL) of compound S19 (1.65 g, 2.667 mmol) was added trifluoroacetic acid (15 mL) dropwise at 0° C. The reaction mixture was stirred for 1 hour, then concentrated under vacuo and azeotroped with toluene 3 times to remove residual trifluoroacetic acid to afford the free carboxylic acid, which was used in subsequent reaction without further purification. To a CH₂Cl₂ solution (30 mL) of the free carboxylic acid were added HATU (1.318 g, 3.467 mmol) and trimethylamine (269 mg, 2.667 mmol) subsequently. After the mixture was stirred for 30 minutes, 5-Amino-2,2-difluoro-1,3-benzodioxole (600 mg, 3.467 mmol) was subsequently added and stirred for overnight. The reaction mixture was purified by flash chromatography concentrated under vacuo to afford the Fmoc-protected dipeptide without further purification. The CH₂Cl₂ solution (16 mL) of Fmoc-protected dipeptide was added 4 mL piperidine and stirred for 30 minutes. The reaction mixture was then concentrated under vacuo and purified by flash chromatography to afford colorless oil S20 (783 mg, 37% yield for 3 steps). R_{f}=0.4 (50% EtOAc in Hexane); ¹H NMR (500 MHz, CDCl₃) δ 10.27 (s, 1H), 9.93-8.86 (br, 1H), 7.71 (d, J=1.5 Hz, 1H), 7.38-7.22 (m, 6H), 6.95 (d, J=8.6 Hz, 1H), 5.78-5.57 (br, 2H), 4.60-4.48 (m, 2H), 4.36 (t, J=6.6 Hz, 1H), 4.29-4.23 (m, 1H), 3.86 (dd, J=10.5, 2.7 Hz, 1H), 3.76 (dd, J=10.6, 3.9 Hz, 1H), 1.94-1.83 (m, 1H), 1.78 (ddd, J=14.3, 8.7, 5.4 Hz, 1H), 1.68 (ddd, J=14.2, 8.7, 4.0 Hz, 1H), 0.98 (d, J=4.2 Hz, 3H), 0.97 (d, J=4.3 Hz, 3H); ¹³C NMR (126 MHz, CD₃OD) δ 172.1, 171.3, 144.7, 141.2, 139.1, 135.7, 133.1 (t, ³J_{C—F}=253.1 Hz), 129.3, 128.7, 128.6, 116.6, 110.5, 103.9, 86.5, 84.4, 74.4, 70.1, 42.0, 25.8, 23.6, 22.3; ¹⁹F NMR (471 MHz, CDCl₃) δ −50.0; HRMS (ESI) for C₂₃H₂₈N₃O₇F₂ (M+H)⁺: calculated 496.1890, found 496.1889.

To the CH₂Cl₂ solution (10 mL) of free carboxylic acid (1.3 mmol, 1 equiv) were added HATU (1.3 mmol, 1.3 equiv) and trimethylamine (1.3 mmol, 1 equiv) subsequently. The reaction mixture was stirred for 30 minutes. S20 (1.3 mmol, 1.3 equiv) was added dropwise. The resulting reaction mixture was stirred overnight, then washed sequentially with saturated NaHCO₃ solution and 0.1 M HCl solution. Then the organic layer was dried over anhydrous Na₂SO₄ and concentrated under vacuo. The reaction mixture was purified by flash chromatography to afford compound 42-compound 52.

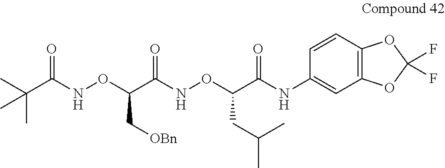 [SMILES: CC(C)C[C@H](ONC(=O)[C@@H](COCc1ccccc1)ONC(=O)C(C)(C)C)C(=O)Nc1ccc2c(c1)OC(F)(F)O2]

Compound 42: ¹H NMR (500 MHz, CDCl₃) δ 12.18 (s, 1H), 10.17 (s, 1H), 8.79 (s, 1H), 7.71 (d, J=1.9 Hz, 1H), 7.40-7.30 (m, 5H), 7.28-7.26 (m, 1H), 6.96 (d, J=8.7 Hz, 1H), 4.64-4.54 (m, 2H), 4.48 (dd, J=8.6, 2.7 Hz, 1H), 4.35 (dd, J=8.5, 5.0 Hz, 1H), 4.03 (dd, J=11.3, 2.8 Hz, 1H), 3.84 (dd, J=11.2, 8.8 Hz, 1H), 2.04-1.94 (m, 1H), 1.85-1.74 (m, 2H), 1.13 (s, 9H), 1.05 (d, J=6.6 Hz, 3H), 1.00 (d, J=6.7 Hz, 3H); ¹³C NMR (126 MHz, CDCl₃) δ 179.6, 169.7, 166.9, 143.9, 140.1, 137.3, 134.4, 131.9 (t, ¹J_{C—F}=254.8 Hz), 128.8, 128.4, 128.1, 114.7, 109.3, 102.8, 86.2, 85.3, 74.1, 69.2, 40.7, 38.2, 27.0, 24.9, 23.4, 21.7; ¹⁹F NMR (471 MHz, CDCl₃) δ −50.1; HRMS (ESI) for C₂₈H₃₆N₃O₈F₂ (M+H)⁺: calculated 580.2465, found 580.2465.

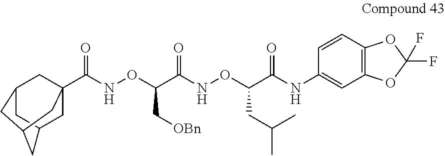 [SMILES: CC(C)C[C@H](ONC(=O)[C@@H](COCc1ccccc1)ONC(=O)C12CC3CC(CC(C3)C1)C2)C(=O)Nc1ccc2c(c1)OC(F)(F)O2]

Compound 43: ¹H NMR (500 MHz, CDCl₃) δ 12.21 (s, 1H), 10.18 (s, 1H), 8.70 (s, 1H), 7.72 (d, J=2.1 Hz, 1H), 7.40-7.30 (m, 5H), 7.29-7.26 (m, 1H), 6.96 (d, J=8.7 Hz, 1H), 4.65-4.53 (m, 2H), 4.48 (dd, J=8.6, 2.9 Hz, 1H), 4.35 (dd, J=8.8, 4.7 Hz, 1H), 4.02 (dd, J=11.3, 2.9 Hz, 1H), 3.84 (dd, J=11.2, 8.7 Hz, 1H), 2.05-1.93 (m, 4H), 1.84-1.76 (m, 2H), 1.76-1.62 (m, 12H), 1.05 (d, J=6.6 Hz, 3H), 1.00 (d, J=6.7 Hz, 3H); ¹³C NMR (126 MHz, CDCl₃) δ 179.0, 169.8, 167.0, 143.9, 140.1, 137.4, 134.5, 132.0 (t, ¹J_{C—F}=254.7 Hz), 128.9, 128.4, 128.2, 114.8, 109.3, 102.9, 86.3, 85.3, 74.1, 69.2, 40.8, 40.3, 38.7, 36.3, 27.8, 25.0, 23.4, 21.8; ¹⁹F NMR (471 MHz, CDCl₃) δ −50.1; HRMS (ESI) for C₃₄H₄₂N₃O₈F₂ (M+H)⁺: calculated 658.2934, found 658.2934.

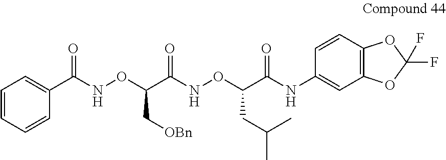 [SMILES: CC(C)C[C@H](ONC(=O)[C@@H](COCc1ccccc1)ONC(=O)c1ccccc1)C(=O)Nc1ccc2c(c1)OC(F)(F)O2]

Compound 44: ¹H NMR (500 MHz, CDCl₃) δ 12.19 (s, 1H), 10.23 (s, 1H), 9.26 (s, 1H), 7.70 (d, J=1.6 Hz, 1H), 7.61 (d, J=7.8 Hz, 2H), 7.58 (t, J=7.5 Hz, 1H), 7.44 (t, J=7.7 Hz, 2H), 7.39-7.30 (m, 5H), 7.29-7.23 (m, 1H), 6.95 (d, J=8.6 Hz, 1H), 4.69-4.56 (m, 3H), 4.42 (dd, J=8.6, 5.0 Hz, 1H), 4.09 (dd, J=11.3, 2.8 Hz, 1H), 3.91 (dd, J=11.1, 8.9 Hz, 1H), 2.08-1.97 (m, 1H), 1.88-1.78 (m, 2H), 1.07 (d, J=6.6 Hz, 3H), 1.01 (d, J=6.7 Hz, 3H); ¹³C NMR (126 MHz, CDCl₃) δ 169.8, 168.8, 167.1, 143.9, 140.1, 137.3, 134.4, 133.3, 131.9 (t, ¹J_{C—F}=255.8 Hz), 129.9, 129.1, 128.9, 128.4, 128.1, 127.3, 114.7, 109.3, 102.8, 86.4, 85.5, 74.0, 69.0, 40.7, 24.9, 23.4, 21.7; ¹⁹F NMR (471 MHz, CDCl₃) δ −50.1; HRMS (ESI) for C₃₀H₃₂N₃O₈F₂ (M+H)⁺: calculated 600.2152, found 600.2150.

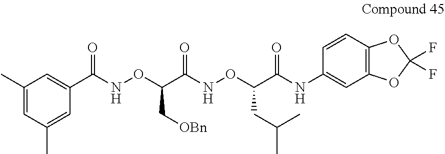 [SMILES: Cc1cc(C)cc(C(=O)NO[C@H](COCc2ccccc2)C(=O)NO[C@@H](CC(C)C)C(=O)Nc2ccc3c(c2)OC(F)(F)O3)c1]

Compound 45: ¹H NMR (308 K, 500 MHz, CDCl₃) δ 12.15 (s, 1H), 10.21 (s, 1H), 9.12 (s, 1H), 7.70 (d, J=2.0 Hz, 1H), 7.34-7.27 (m, 5H), 7.25 (s, 1H), 7.22 (s, 2H), 7.19 (s, 1H), 6.94 (d, J=8.6 Hz, 1H), 4.66-4.55 (m, 3H), 4.41 (dd, J=9.0, 4.4 Hz, 1H), 4.08 (dd, J=11.4, 2.9 Hz, 1H), 3.92 (dd, J=11.3, 8.7 Hz, 1H), 2.33 (s, 6H), 2.07-1.98 (m, 1H), 1.87-1.76 (m, 2H), 1.06 (d, J=6.6 Hz, 3H), 1.02 (d, J=6.7 Hz, 3H); ¹³C NMR (126 MHz, CDCl₃) δ 169.8, 169.3, 167.2, 143.9, 140.0, 139.0, 137.4, 135.0, 134.4, 131.9 (t, ¹J_{C—F}=254.5 Hz), 129.8, 128.8, 128.4, 128.1, 126.0, 125.0, 114.9, 109.3, 102.9, 86.3, 85.7, 74.0, 69.2, 40.7, 24.9, 23.4, 21.7, 21.3; ¹⁹F NMR (471 MHz, CDCl₃) δ −50.6; HRMS (ESI) for C₃₂H₃₆N₃O₈F₂ (M+H)⁺: calculated 628.2465, found 628.2463.

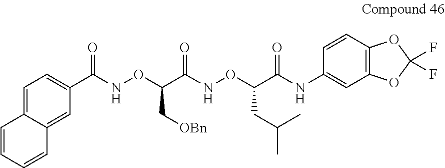 [SMILES: CC(C)C[C@H](ONC(=O)[C@@H](COCc1ccccc1)ONC(=O)c1ccc2ccccc2c1)C(=O)Nc1ccc2c(c1)OC(F)(F)O2]

Compound 46: ¹H NMR (500 MHz, CDCl₃) δ 12.23 (s, 1H), 10.24 (s, 1H), 9.42 (s, 1H), 8.17 (s, 1H), 7.91-7.76 (m, 3H), 7.70 (d, J=2.0 Hz, 1H), 7.65-7.54 (m, 3H), 7.39-7.30 (m, 5H), 7.28-7.26 (m, 1H), 6.94 (d, J=8.7 Hz, 1H), 4.69 (dd, J=8.8, 2.7 Hz, 1H), 4.68-4.59 (m, 2H), 4.45 (dd, J=8.5, 5.0 Hz, 1H), 4.13 (dd, J=11.3, 2.7 Hz, 1H), 3.94 (dd, J=11.2, 8.9 Hz, 1H), 2.11-1.98 (m, 1H), 1.90-1.77 (m, 2H), 1.08 (d, J=6.6 Hz, 3H), 1.03 (d, J=6.7 Hz, 3H); ¹³C NMR (126 MHz, CDCl₃) δ 169.9, 169.0, 167.2, 143.9, 140.1, 137.4, 135.6, 134.5, 132.6, 132.0 (t, ¹J_{C—F}=255.8 Hz), 129.3, 128.9, 128.8, 128.6, 128.5, 128.2, 128.1, 127.5, 127.1, 123.0, 114.8, 109.4, 102.9, 86.4, 85.8, 74.1, 69.3, 40.8, 25.0, 23.5, 21.8; ¹⁹F NMR (471 MHz, CDCl₃) δ −50.1 HRMS (ESI) for C₃₄H₃₄N₃O₈F₂ (M+H)⁺: calculated 650.2308, found 650.2309.

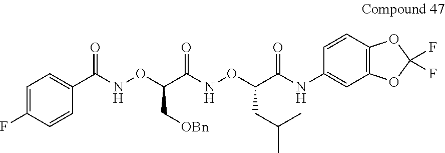 [SMILES: CC(C)C[C@H](ONC(=O)[C@@H](COCc1ccccc1)ONC(=O)c1ccc(F)cc1)C(=O)Nc1ccc2c(c1)OC(F)(F)O2]

Compound 47: ¹H NMR (500 MHz, CDCl₃) δ 12.48-11.60 (br, 1H), 10.21 (s, 1H), 9.59-8.79 (br, 1H), 7.69 (d, J=2.0 Hz, 1H), 7.65-7.60 (m, 2H), 7.40-7.31 (m, 4H), 7.28-7.25 (m, 2H) 7.14-7.07 (m, 2H), 6.95 (d, J=8.7 Hz, 1H), 4.66-4.56 (m, 3H), 4.41 (dd, J=8.6, 5.0 Hz, 1H), 3.86 (dd, J=10.5, 2.7 Hz, 1H), 3.76 (dd, J=10.6, 3.9 Hz, 1H), 2.06-1.96 (m, 1H), 1.86-1.76 (m, 2H), 1.06 (d, J=6.6 Hz, 3H), 1.01 (d, J=6.7 Hz, 3H); ¹³C NMR (126 MHz, CDCl₃) δ 169.8, 167.14, 167.12, 165.8 (d, ¹J_{C—F}=255.2 Hz), 143.9, 140.2, 137.4, 134.5, 133.0 (t, ¹J_{C—F}=254.8 Hz), 130.0 (d, ³J_{C—F}=9.4 Hz), 129.0, 128.5, 128.3, 126.1, 116.4 (d, ²J_{C—F}=22.0 Hz), 114.8, 109.4, 102.9, 86.4, 85.6, 74.1, 69.1, 40.7, 25.0, 23.5, 21.7; ¹⁹F NMR (471 MHz, CDCl₃) δ −50.1 (2F), −104.5 (1F); HRMS (ESI) for C₃₀H₃₁N₃O₈F₃ (M+H)⁺: calculated 618.2058, found 618.2058.

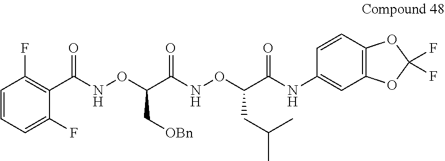 [SMILES: CC(C)C[C@H](ONC(=O)[C@@H](COCc1ccccc1)ONC(=O)c1c(F)cccc1F)C(=O)Nc1ccc2c(c1)OC(F)(F)O2]

Compound 48: ¹H NMR (310 K, 500 MHz, CDCl₃) δ 11.57 (s, 1H), 10.19 (s, 1H), 9.07 (s, 1H), 7.70 (s, 1H), 7.51-7.40 (m, 1H), 7.34-7.27 (m, 6H), 7.03-6.90 (m, 3H), 4.69-4.63 (m, 1H), 4.63-4.55 (m, 2H), 4.43 (dd, J=9.0, 3.8 Hz, 1H), 4.07 (dd, J=11.6, 2.0 Hz, 1H), 3.91 (dd, J=10.8, 8.9 Hz, 1H), 2.08-1.91 (m, 1H), 1.88-1.75 (m, 2H), 1.05 (d, J=6.6 Hz, 3H), 1.00 (d, J=6.6 Hz, 3H); ¹³C NMR (126 MHz, CDCl₃) δ 169.7, 167.1, 161.2, 160.5 (dd, ¹J_{C—F}=255.2 Hz, ³J_{C—F}=5.7 Hz), 143.9, 140.1, 137.2, 134.5, 133.9 (t, ³J_{C—F}=10.7 Hz), 132.0 (t, ¹J_{C—F}=254.6 Hz), 128.8, 128.4, 128.1, 114.8, 112.6 (dd, ²J_{C—F}=21.5 Hz, ⁴J_{C—F}=3.6 Hz), 109.6 (t, ²J_{C—F}=18.7 Hz), 109.3, 102.90, 86.6, 85.5, 74.0, 68.8, 40.8, 25.0, 23.4, 21.7; ¹⁹F NMR (471 MHz, CDCl₃) δ −50.1 (2F), −110.3 (2F); HRMS (ESI) for C₃₀H₃₀N₃O₈F₄ (M+H)⁺: calculated 636.1963, found 636.1964.

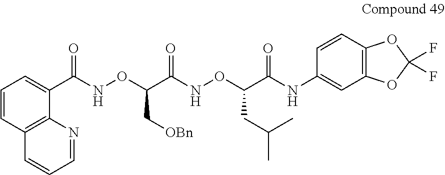 [SMILES: CC(C)C[C@H](ONC(=O)[C@@H](COCc1ccccc1)ONC(=O)c1cccc2cccnc12)C(=O)Nc1ccc2c(c1)OC(F)(F)O2]

Compound 49: ¹H NMR (500 MHz, CDCl₃) δ 14.12 (s, 1H), 12.71 (s, 1H), 10.32 (s, 1H), 8.77 (dd, J=7.4, 1.4 Hz, 1H), 8.69 (dd, J=4.3, 1.8 Hz, 1H), 8.31 (dd, J=8.3, 1.7 Hz, 1H), 8.04 (dd, J=8.1, 1.3 Hz, 1H), 7.73 (t, J=7.8 Hz, 1H), 7.66 (d, J=2.1 Hz, 1H), 7.49 (dd, J=8.2, 4.3 Hz, 1H), 7.42-7.30 (m, 5H), 7.22 (dd, J=8.7, 2.1 Hz, 1H), 6.89 (d, J=8.7 Hz, 1H), 4.79 (dd, J=8.2, 2.9 Hz, 1H), 4.71-4.65 (m, 2H), 4.50 (dd, J=8.1, 5.5 Hz, 1H), 4.15 (dd, J=11.2, 2.9 Hz, 1H), 4.00 (dd, J=11.2, 8.2 Hz, 1H), 2.15-2.04 (m, 1H), 1.91-1.81 (m, 2H), 1.14 (d, J=6.6 Hz, 3H), 1.06 (d, J=6.7 Hz, 3H); ¹³C NMR (126 MHz, CDCl₃) δ 170.0, 167.8, 167.5, 150.0, 144.9, 143.8, 140.0, 138.3, 137.8, 134.6, 134.5, 133.4, 131.9 (t, ¹J_{C—F}=254.5 Hz), 128.73, 128.71, 128.1, 127.9, 127.0, 126.5, 121.7, 114.8, 109.2, 102.8, 86.3, 85.6, 73.9, 69.2, 40.8, 25.1, 23.5, 21.8; ¹⁹F NMR (471 MHz, CDCl₃) δ −50.1, HRMS (ESI) for C₃₃H₃₃N₄O₈F₂ (M+H)⁺: calculated 651.2261, found 651.2256.

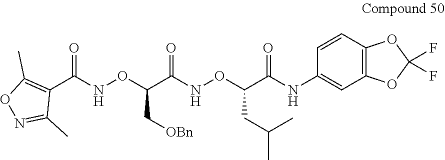 [SMILES: Cc1noc(C)c1C(=O)NO[C@H](COCc1ccccc1)C(=O)NO[C@@H](CC(C)C)C(=O)Nc1ccc2c(c1)OC(F)(F)O2]

Compound 50: ¹H NMR (500 MHz, CDCl₃) δ 11.81 (s, 1H), 10.18 (s, 1H), 8.88 (s, 1H), 7.69 (s, 1H), 7.40-7.29 (m, 5H), 7.29-7.25 (m, 1H), 6.96 (d, J=8.6 Hz, 1H), 4.65-4.55 (m, 3H), 4.41 (dd, J=8.6, 2.9 Hz, 1H), 4.11 (dd, J=11.0, 2.0 Hz, 1H), 3.94 (t, J=10.0 Hz, 1H), 2.52 (s, 3H), 2.27 (s, 3H), 2.06-1.94 (m, 1H), 1.87-1.79 (m, 2H), 1.05 (d, J=6.5 Hz, 3H), 1.01 (d, J=6.6 Hz, 3H); ¹³C NMR (126 MHz, CDCl₃) δ 173.8, 171.3, 170.0, 167.0, 157.7, 143.9, 140.2, 137.1, 134.4, 132.0 (t, ¹J_{C—F}=255.1 Hz), 128.9, 128.7, 128.3, 114.7, 109.4, 108.1, 102.8, 86.4, 85.1, 74.2, 69.3, 40.6, 25.0, 23.4, 21.6, 13.3, 11.8; ¹⁹F NMR (471 MHz, CDCl₃) δ −50.1; HRMS (ESI) for C₂₉H₃₃N₄O₉F₂ (M+H)⁺: calculated 619.2210, found 619.2209.

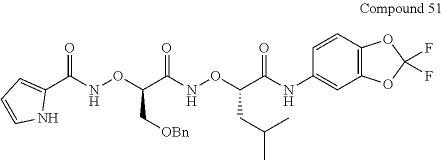 [SMILES: CC(C)C[C@H](ONC(=O)[C@@H](COCc1ccccc1)ONC(=O)c1ccc[nH]1)C(=O)Nc1ccc2c(c1)OC(F)(F)O2]

Compound 51: ¹H NMR (500 MHz, CDCl₃) δ 12.19 (s, 1H), 10.23 (s, 1H), 9.29 (s, 1H), 8.90 (s, 1H), 7.68 (d, J=1.8 Hz, 1H), 7.40-7.32 (m, 5H), 7.24 (dd, J=8.7, 1.9 Hz, 1H), 7.03 (s, 1H), 6.94 (d, J=8.6 Hz, 1H), 6.50 (s, 1H), 6.28-6.20 (m, 1H), 4.66-4.55 (m, 3H), 4.42 (dd, J=7.6, 5.9 Hz, 1H), 4.11 (dd, J=11.3, 2.7 Hz, 1H), 3.88 (dd, J=11.2, 8.7 Hz, 1H), 2.05-1.95 (m, 1H), 1.85-1.77 (m, 2H), 1.05 (d, J=6.6 Hz, 3H), 1.01 (d, J=6.7 Hz, 3H); ¹³C NMR (126 MHz, CDCl₃) δ 169.9, 167.3, 163.3, 143.9, 140.2, 137.5, 134.4, 132.0 (t, ¹J_{C—F}=254.6 Hz), 128.9, 128.5, 128.2, 124.2, 121.0, 114.8, 111.8, 111.1, 109.4, 102.9, 86.2, 85.8, 74.1, 69.1, 40.8, 25.0, 23.5, 21.8; ¹⁹F NMR (471 MHz, CDCl₃) δ −50.1; HRMS (ESI) for C₂₈H₃₁N₄O₈F₂ (M+H)⁺: calculated 589.2104, found 589.2108.

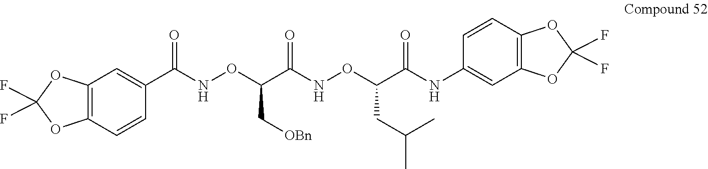 [SMILES: CC(C)C[C@H](ONC(=O)[C@@H](COCc1ccccc1)ONC(=O)c1ccc2c(c1)OC(F)(F)O2)C(=O)Nc1ccc2c(c1)OC(F)(F)O2]

Compound 52: ¹H NMR (500 MHz, CDCl₃) δ 11.92 (s, 1H), 10.19 (s, 1H), 9.62 (s, 1H), 7.65 (d, J=2.0 Hz, 1H), 7.34-7.29 (m, 6H), 7.24 (dd, J=8.6, 2.0 Hz, 2H), 7.05 (d, J=8.2 Hz, 1H), 6.93 (d, J=8.7 Hz, 1H), 4.65-4.62 (m, 3H), 4.39 (d, J=6.8 Hz, 1H), 4.06 (dd, J=11.2, 2.8 Hz, 1H), 3.90 (dd, J=11.2, 8.2 Hz, 1H), 2.05-1.92 (m, 1H), 1.85-1.73 (m, 2H), 1.03 (d, J=6.6 Hz, 3H), 0.99 (d, J=6.7 Hz, 3H); ¹³C NMR (126 MHz, CDCl₃) δ 169.8, 169.8, 167.1, 147.1, 144.2, 143.8, 140.1, 137.1, 134.2, 131.9 (t, ¹J_{C—F}=255.0 Hz), 131.7 (t, ¹J_{C—F}=258.3 Hz), 128.8, 128.5, 128.2, 126.0, 123.8, 114.8, 109.7, 109.3, 109.0, 102.8, 86.3, 85.5, 74.5, 69.0, 40.6, 24.9, 23.3, 21.6; ¹⁹F NMR (471 MHz, CDCl₃) δ −49.6 (s, 2H), −50.1 (s, 2H); HRMS (ESI) for C₃₁H₃₀N₃O₁₀F₄ (M+H)⁺: calculated 680.1862, found 680.1857.

Compound 53 was synthesized according to the general procedures described below (Scheme 16).

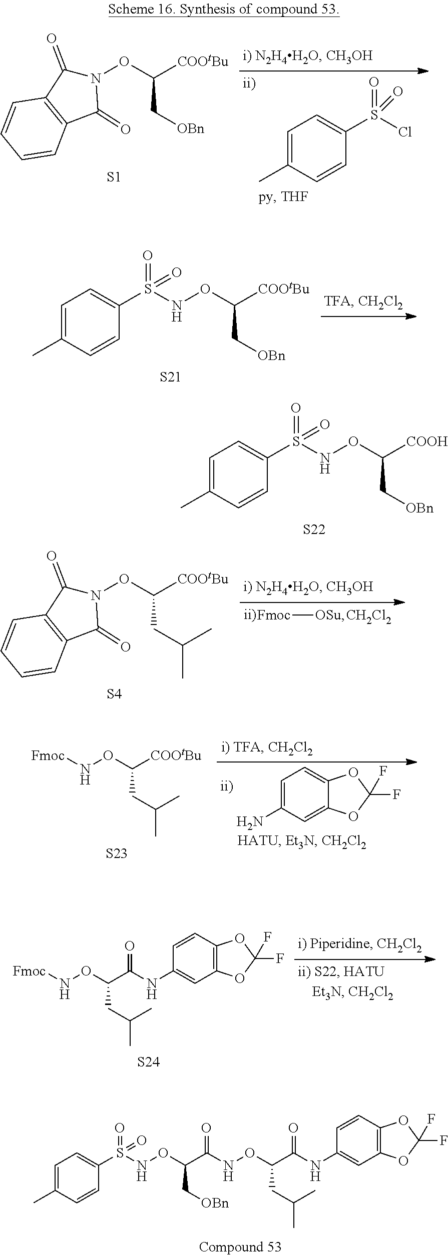

S2 was synthesized from S1 (100 mg, 252 μmol) according to the Scheme 1 without further purification. To the solution of S2 in 3 mL anhydrous THF on ice bath was added pyridine (40 mg, 503 μmol). Tosyl chloride was then added (48 mg, 252 μmol). The reaction mixture was stirred overnight, then concentrated under vacuo and purified by flash chromatography to afford colorless oil S21 (83 mg, 78%). R_{f}=0.4 (50% EtOAc in Hexane). ¹H NMR (500 MHz, CDCl₃) δ 7.81-7.77 (m, 3H), 7.35-7.21 (m, 6H), 4.71 (t, J=3.80 Hz, 1H), 4.56 (d, J=12.0 Hz, 1H), 4.44 (d, J=12.0 Hz, 1H), 3.75 (d, J=3.9 Hz, 2H), 2.40 (s, 3H), 1.45 (s, 9H); ¹³C NMR (126 MHz, CDCl₃) δ 168.1, 144.8, 137.6, 133.4, 129.7, 128.80, (128.79), 128.4, 127.8, 84.5, 82.8, 73.2, 68.8, 28.1, 21.7; HRMS (ESI) for C₂₁H₂₇NNaO₆ (M+Na)⁺: calculated 444.1451, found 444.1448.

To a CH₂Cl₂ solution (0.5 mL) of compound S21 (17 mg, 40 μmol) was added trifluoroacetic acid (0.5 mL) dropwise at 0° C. The reaction mixture was stirred for 1 hour, then concentrated under vacuo and azeotroped with toluene 3 times to remove residual trifluoroacetic acid to afford free carboxylic acid S22, which was used in subsequent reaction without further purification.

To starting material S4 (500 mg, 1.477 mmol) in CH₃OH solution (15 mL) was added N₂H₄·H₂O (657 mg, 13 mmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To the free amine in anhydrous CH₂Cl₂ (15 mL), Fmoc-OSu (222 mg, 4.43 mmol) was added. The reaction was stirred overnight and then concentrated under vacuo. The reaction mixture was purified by flash chromatography to afford white solid S23 (498 mg, 79% yield for 2 steps). R_{f}=0.3 (10% EtOAc in Hexane); ¹H NMR (500 MHz, CDCl₃) δ 8.39 (s, 1H), 7.77 (d, J=7.6 Hz, 2H), 7.62 (dd, J=7.2, 4.4 Hz, 2H), 7.41 (t, J=7.5 Hz, 2H), 7.31 (t, J=7.5 Hz, 2H), 4.54-4.44 (m, 2H), 4.41 (dd, J=9.3, 4.2 Hz, 1H), 4.27 (t, J=7.2 Hz, 1H), 2.05-1.95 (m, 1H), 1.80-1.71 (m, 1H), 1.61-1.57 (m, 1H), 1.54 (s, 9H), 1.04 (d, J=6.6 Hz, 3H), 1.00 (d, J=6.7 Hz, 3H); ¹³C NMR (126 MHz, CDCl₃) δ 171.6, 157.1, 143.6, 143.5, 141.29, 141.27, 127.8, 127.10, 127.07, 125.1, 120.1, 82.9, 82.1, 67.6, 47.0, 39.9, 28.1, 24.7, 23.1, 21.8; HRMS (ESI) for C₂₅H₃₂NO₅ (M+H)⁺: calculated 448.2094, found 448.2094.

To a CH₂Cl₂ solution (5 mL) of compound S23 (392 mg, 922 μmol) was added trifluoroacetic acid (5 mL) dropwise at 0° C. The reaction mixture was stirred for 1 hour, then concentrated under vacuo and azeotroped with toluene 3 times to remove residual trifluoroacetic acid to afford free carboxylic acid. To a CH₂Cl₂ solution (10 mL) of the free carboxylic acid were added HATU (386 mg, 1.014 mmol) and trimethylamine (186 mg, 1.844 mmol) subsequently. After the mixture was stirred for 30 minutes, 5-Amino-2,2-difluoro-1,3-benzodioxole (176 mg, 1.014 mmol) was subsequently added. The reaction mixture was then concentrated under vacuo and purified by flash chromatography to afford white solid S24 (82 mg, 17% yield for 2 steps). ¹H NMR (500 MHz, CDCl₃) δ 9.97 (s, 1H), 7.86 (s, 1H), 7.75 (dd, J=6.9, 5.0 Hz, 2H), 7.69-7.66 (br, 1H), 7.51 (d, J=7.5 Hz, 2H), 7.43-7.34 (m, 2H), 7.29-7.19 (m, 3H), 7.75 (d, J=8.7 Hz, 1H), 4.61-4.50 (m, 2H), 4.31 (dd, J=9.0, 4.4 Hz, 1H), 4.19 (t, J=6.6 Hz, 1H), 1.91-1.79 (m, 1H), 1.79-1.67 (m, 2H), 0.97 (t, J=7.0 Hz, 6H); ¹³C NMR (126 MHz, CDCl₃) δ 169.7, 158.9, 143.7, 142.9, 142.8, 141.31, 141.26, 139.9, 134.1, 131.7 (t, ¹J_{C—F}=254.9 Hz), 128.01, 127.95, 127.20, 127.15, 124.72, 124.68, 120.14, 120.11, 114.5, 109.1, 102.6, 86.5, 68.3, 46.8, 40.6, 24.8, 23.2, 21.6; ¹⁹F NMR (471 MHz, CDCl₃) δ −50.0; HRMS (ESI) for C₂₈H₂₇N₂O₆F₂ (M+H)⁺: calculated 525.1832, found 525.1827.

The CH₂Cl₂ solution (2 mL) of S24 (82 mg, 150 μmol) was added 0.3 mL piperidine and stirred for 30 minutes. The reaction mixture was then concentrated under vacuo and purified by flash chromatography to afford free amine without further purification. To a CH₂Cl₂ solution (1 mL) of S22 were added HATU (15 mg, 40 μmol) and trimethylamine (4.4 mg, 44 μmol) subsequently. After the mixture was stirred for 30 minutes, the free amine (11 mg, 36 μmol) was subsequently added and stirred for overnight. The reaction mixture was then concentrated under vacuo and purified by flash chromatography to afford white solid compound 53. R_{f}=0.3 (30% EtOAc in Hexane); ¹H NMR (310 K, 500 MHz, CDCl₃) δ 10.06 (s, 1H), 9.61 (s, 1H), 7.75-7.70 (m, 3H), 7.67 (s, 1H), 7.33-7.26 (m, 5H), 7.24-7.18 (m, 3H), 7.00-6.89 (m, 1H), 4.61 (t, J=3.8 Hz, 1H), 4.47 (s, 2H), 4.61 (t, J=6.6 Hz, 1H), 3.86 (dd, J=11.0, 3.3 Hz, 1H), 3.80 (dd, J=11.0, 5.1 Hz, 1H), 2.41 (s, 3H), 1.93-1.81 (m, 1H), 1.79-1.72 (m, 2H), 0.96 (d, J=3.6 Hz, 3H), 0.95 (d, J=3.6 Hz, 3H); ¹³C NMR (126 MHz, CDCl₃) δ 169.9, 168.8, 145.9, 143.9, 140.2, 137.1, 134.1, 132.7, 131.9 (t, ¹J_{C—F}=255.1 Hz), 130.1, 128.8, 128.7, 128.3, 127.9, 115.0, 103.4, 103.0, 86.5, 84.4, 73.8, 68.0, 40.8, 24.9, 23.3, 21.8, 21.7; ¹⁹F NMR (471 MHz, CDCl₃) δ −50.0; HRMS (ESI) for C₃₀H₃₄N₃O₉F₂S (M+H)⁺: calculated 650.1978, found 650.1978.

Compound 54 was synthesized according to the general procedures described below (Scheme 17).

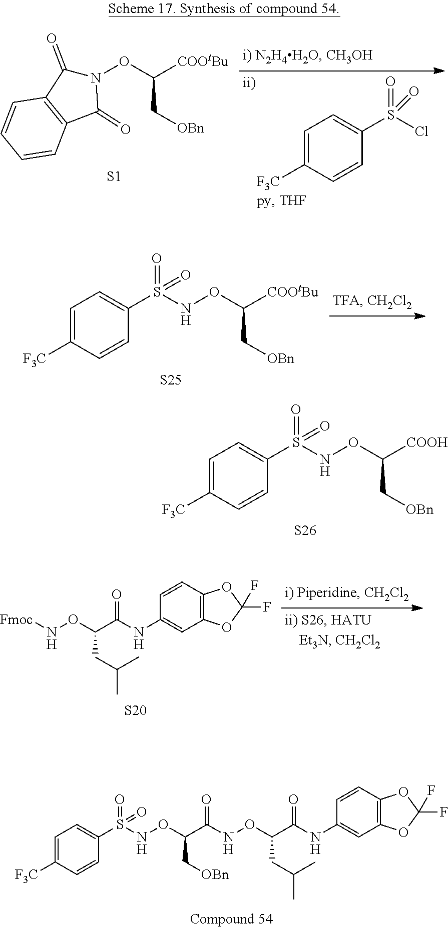

S2 was synthesized from S1 (200 mg, 503 μmol) according to the Scheme 1 without further purification. To the solution of S2 (95 mg, 355 μmol) in 4 mL anhydrous THF on ice bath was added pyridine (84 mg, 1.065 mmol). 4-trifluoromethyl benzenesulfonyl chloride was then added (87 mg, 355 μmol). The reaction mixture was stirred overnight, then concentrated under vacuo and purified by flash chromatography to afford colorless oil S25 (155 mg, 92%). R_{f}=0.7 (30% EtOAc in Hexane); ¹H NMR (500 MHz, CDCl₃) δ 8.21-7.94 (m, 3H), 7.65 (d, J=7.7 Hz, 2H), 7.34-7.26 (m, 5H), 4.74 (dd, J=5.1, 2.7 Hz, 1H), 4.57 (d, J=11.9 Hz, 1H), 4.47 (d, J=11.9 Hz, 1H), 3.79-3.71 (m, 2H), 1.46 (s, 9H); ¹³C NMR (126 MHz, CDCl₃) δ 168.0, 139.8, 137.4, 135.4 (q, ²J_{C—F}=35.0 Hz), 129.5, 128.5, 128.0, 127.9, 126.1 (q, ³J_{C—F}=3.3 Hz), 123.2 (q, ¹J_{C—F}=273.1 Hz), 84.6, 83.2, 73.4, 68.7, 28.1; ¹⁹F NMR (471 MHz, CDCl₃) δ −63.2; HRMS (ESI) for C₂₂H₂₄NNaO₆F₆S (M+Na)⁺: calculated 498.1169, found 498.1168.

To a CH₂Cl₂ solution (0.5 mL) of compound S25 (19 mg, 40 μmol) was added trifluoroacetic acid (0.5 mL) dropwise at 0° C. The reaction mixture was stirred for 1 hour, then concentrated under vacuo and azeotroped with toluene 3 times to remove residual trifluoroacetic acid to afford free carboxylic acid 526, which was used in subsequent reaction without further purification.

The CH₂Cl₂ solution (2 mL) of S20 (82 mg, 150 μmol) was added 0.3 mL piperidine and stirred for 30 minutes. The reaction mixture was then concentrated under vacuo and purified by flash chromatography to afford free amine without further purification. To a CH₂Cl₂ solution (1 mL) of S26 were added HATU (15 mg, 40 μmol) and trimethylamine (4.4 mg, 44 μmol) subsequently. After the mixture was stirred for 30 minutes, the free amine (11 mg, 36 μmol) was subsequently added and stirred for overnight. The reaction mixture was then concentrated under vacuo and purified by flash chromatography to afford white solid compound 54. R_{f}=0.5 (20% EtOAc in Hexane); ¹H NMR (600 MHz, CD₃CN) δ 11.69 (s, 1H), 10.85 (s, 1H), 10.27 (s, 1H), 8.07 (d, J=8.0 Hz, 2H), 7.93 (d, J=8.0 Hz, 2H), 7.76-7.68 (m, 1H), 7.38-7.16 (m, 7H), 4.62 (br, 1H), 4.54-4.32 (m, 3H), 3.60 (d, J=4.5 Hz, 2H), 1.96-1.83 (m, 1H), 1.76-1.68 (m, 1H), 1.67-1.55 (m, 1H), 0.98 (d, J=6.4 Hz, 3H), 0.95 (d, J=6.4 Hz, 3H); ¹³C NMR (150 MHz, CD₃CN) δ 171.2, 169.1, 144.8, 141.5, 141.0, 139.3, 136.2, 136.0 (q, ²J_{C—F}=32.8 Hz), 133.2 (t, ¹J_{C—F}=252.2 Hz), 130.9, 129.7, 129.1, 129.0, 127.7 (q, ³J_{C—F}=3.8 Hz), 125.1 (q, ¹J_{C—F}=251.1 Hz), 116.5, 111.1, 104.0, 87.1, 85.8, 74.3, 69.7, 41.9, 25.9, 23.8, 22.4; ¹⁹F NMR (471 MHz, CD₃OD) δ −52.3 (2F), −64.7 (3F); HRMS (ESI) for C₃₀H₃₁N₃O₉F₅S (M+H)⁺: calculated 704.1696, found 704.1690.

Compound 55 was synthesized according to the general procedures described below (Scheme 18).

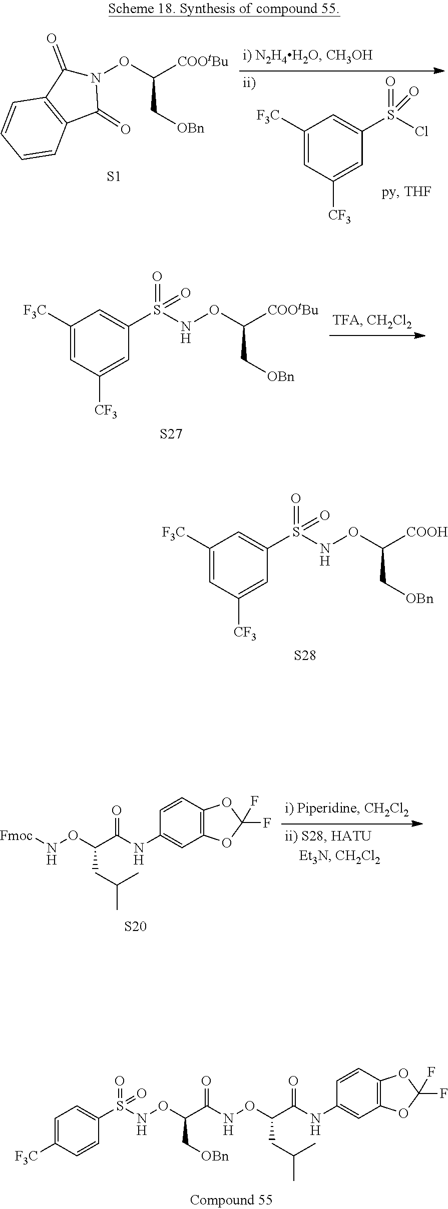

S2 was synthesized from S1 (200 mg, 503 μmol) according to the Scheme 1 without further purification. To the solution of S2 (67 mg, 252 μmol) in 4 mL anhydrous THF on ice bath was added pyridine (60 mg, 755 μmol). 3,5-di(trifluoromethyl)-benzenesulfonyl chloride (79 mg, 252 μmol) was then added. The reaction mixture was stirred overnight, then concentrated under vacuo and purified by flash chromatography to afford colorless oil S27 (74 mg, 92%). R_{f}=0.6 (15% EtOAc in Hexane); ¹H NMR (500 MHz, CDCl₃) δ 8.41 (s, 2H), 8.18 (s, 1H), 8.11 (s, 1H), 7.35-7.20 (m, 5H), 4.75 (t, J=3.6 Hz, 1H), 4.55 (d, J=12.1 Hz, 1H), 4.47 (d, J=12.1 Hz, 1H), 3.72 (d, J=3.7 Hz, 2H), 1.45 (s, 9H); ¹³C NMR (126 MHz, CDCl₃) δ 169.9, 139.1, 137.2, 133.0 (q, ²J_{C—F}=34.6 Hz), 129.3 (q, ³J_{C—F}=3.0 Hz), 128.5, 128.0, 127.8, 127.5-127.4 (m, due to C—F splitting), 122.5 (q, ¹J_{C—F}=273.4 Hz), 84.8, 83.5, 73.5, 68.6, 28.1; ¹⁹F NMR (471 MHz, CDCl₃) δ −63.0; HRMS (ESI) for C₂₂H₂₃N₃NaO₆F₆S (M+Na)⁺: calculated 566.1042, found 566.1035.

To a CH₂Cl₂ solution (0.5 mL) of compound S27 (22 mg, 40 μmol) was added trifluoroacetic acid (0.5 mL) dropwise at 0° C. The reaction mixture was stirred for 1 hour, then concentrated under vacuo and azeotroped with toluene 3 times to remove residual trifluoroacetic acid to afford free carboxylic acid S28, which was used in subsequent reaction without further purification.

The CH₂Cl₂ solution (2 mL) of S20 (82 mg, 150 μmol) was added 0.3 mL piperidine and stirred for 30 minutes. The reaction mixture was then concentrated under vacuo and purified by flash chromatography to afford free amine without further purification. To a CH₂Cl₂ solution (1 mL) of S22 were added HATU (15 mg, 40 μmol) and trimethylamine (4.4 mg, 44 μmol) subsequently. After the mixture was stirred for 30 minutes, the free amine (11 mg, 36 μmol) was subsequently added and stirred for overnight. The reaction mixture was then concentrated under vacuo and purified by flash chromatography to afford white solid compound 55. R_{f}=0.6 (30% EtOAc in Hexane); ¹H NMR (600 MHz, CD₃CN) δ 10.25 (s, 1H), 9.95 (s, 1H), 9.29 (s, 1H), 8.45 (s, 2H), 8.35 (s, 1H), 7.69 (d, J=1.8 Hz, 1H), 7.31-7.17 (m, 6H), 7.11 (d, J=8.7 Hz, 1H), 4.72-4.63 (m, 1H), 4.50-4.39 (m, 2H), 4.36 (dd, J=9.4, 3.9 Hz, 1H), 3.73 (d, J=4.4 Hz, 2H), 1.93-1.87 (m, 1H), 1.78-1.71 (m, 1H), 1.71-1.63 (m, 1H), 0.99 (d, J=6.6 Hz, 3H), 0.97 (d, J=6.7 Hz, 3H); ¹³C NMR (150 MHz, CD₃CN) δ 170.57, 168.5, 144.3, 140.5, 139.8, 138.6, 135.7, 133.1 (q, ²J_{C—F}=34.5 Hz), 132.6 (t, ¹J_{C—F}=252.2 Hz), 130.3 (q, ³J_{C—F}=3.3 Hz), 129.2, 129.0-128.8 (m, due to C—F splitting), 128.5, 128.4, 123.6 (q, ¹J_{C—F}=272.6 Hz), 115.9, 110.6, 103.4, 86.6, 85.1, 73.8, 69.2, 41.4, 25.4, 23.3, 21.8; ¹⁹F NMR (471 MHz, CDCl₃) δ −49.1 (2F), −61.5 (6F); HRMS (ESI) for C₃₁H₃₀F₈N₃O₉S (M+H)⁺: calculated 772.1570, found 775.1566.

Compound 56 was synthesized according to the general procedures described below (Scheme 19).

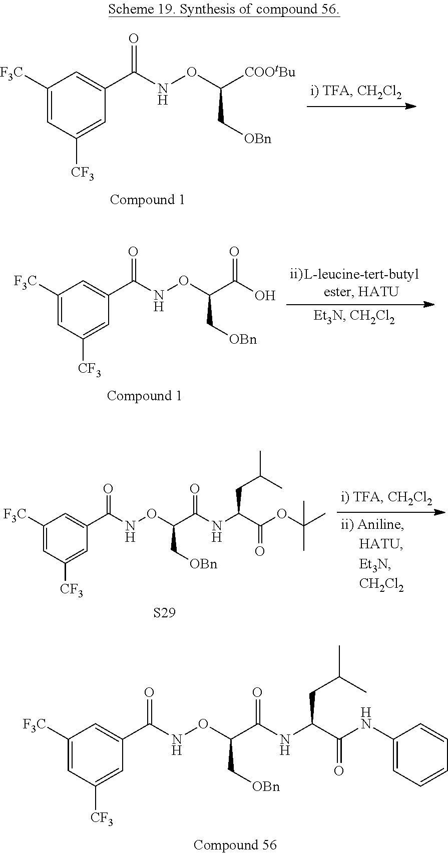

S3 was synthesized from compound 1 (273 mg, 0.54 mmol) according to Scheme 1. The reaction mixture was used in the subsequent reaction without further purification. To a CH₂Cl₂ solution (1 mL) of S3 were added HATU (205 mg, 538 μmol) and trimethylamine (54 mg, 538 μmol). After the mixture was stirred for 10 minutes, L-leucine-methylester hydrochloride (205 mg, 538 μmol) was added. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford S29. (179 mg, 2 steps yield 54%). R_{f}=0.7 (30% EtOAc in Hexane); ¹H NMR (500 MHz, CDCl₃) δ 11.86-9.53 (Br, 1H), 8.22 (s, 1H), 7.30-7.19 (m, 5H), 4.75 (dd, J=7.3, 2.9 Hz, 1H), 4.54 (s, 2H), 4.53-4.49 (m, 1H), 3.98 (dd, J=11.5, 2.8 Hz, 1H), 3.90 (dd, J=11.5, 7.2 Hz, 1H), 1.68-1.53 (m, 3H), 1.43 (s, 9H), 0.88 (d, J=5.8 Hz, 3H), 0.85 (d, J=5.8 Hz, 3H); ¹³C NMR (126 MHz, CDCl₃) δ 171.9, 168.3, 162.9, 137.2, 133.0, 132.2 (q, J=34.0 Hz), 128.6, 128.1, 127.9, 127.5, 125.4, 124.9 (q, J=273.0 Hz), 85.1, 82.1, 73.8, 69.5, 51.5, 41.2, 27.9, 24.9, 22.7, 21.8; ¹⁹F NMR (376 MHz, CD₃OD) δ −63.0; HRMS (ESI) for C₂₉H₃₅F₆N₂O₆ (M+H)⁺: calculated 621.2394, found 621.2441.

To a CH₂Cl₂ solution (1 mL) of compound S29 (179 mg, 288 μmol) was added trifluoroacetic acid (1 mL). The reaction mixture was stirred for 1 hour, then concentrated under vacuo and azeotroped with toluene 3 times to remove residual trifluoroacetic acid to afford free carboxylic acid, which was used in subsequent reaction without further purification. To a CH₂Cl₂ solution (2 mL) of free carboxylic acid were added HATU (109 mg, 288 μmol) and trimethylamine (29 mg, 288 μmol) subsequently. After the mixture was stirred for 15 minutes, aniline (30 mg, 317 μmol) was subsequently added and stirred for overnight. The reaction mixture was then concentrated under vacuo and purified by flash chromatography to afford white solid compound 56 (140 mg, 2 steps 76%). R_{f}=0.5 (30% EtOAc in Hexane); ¹H NMR (500 MHz, CD₃OD) δ 8.36 (s, 2H), 8.16 (s, 1H), 7.54-7.49 (m, 2H), 7.32-7.16 (m, 7H), 7.08 (t, J=7.4 Hz, 1H), 4.73 (t, J=4.1 Hz, 1H), 4.66 (dd, J=10.6, 4.1 Hz, 1H) 4.61-4.52 (m, 2H), 3.95 (d, J=4.3 Hz, 2H), 1.80-1.71 (m, 1H), 1.70-1.59 (m, 1H), 0.91 (d, J=6.1 Hz, 3H), 0.85 (d, J=6.1 Hz, 3H); ¹³C NMR (126 MHz, CDCl₃) δ 171.1, 169.7, 164.4, 137.3, 136.9, 132.9, 132.2 (q, J=35.2 Hz), 128.9, 128.5, 128.1, 127.8, 125.8, 124.9, 122.8 (q, J=273.0 Hz), 120.8, 120.3, 85.8, 74.0, 73.8, 69.5, 53.1, 40.4, 24.9, 22.9, 21.5; ¹⁹F NMR (471 MHz, CD₃OD) δ −63.0; HRMS (ESI) for C₃₂H₃₁F₆N₃O₅ (M+H)⁺: calculated 640.2241, found 640.2242.

Compound 57 was synthesized according to the general procedures described below (Scheme 20).

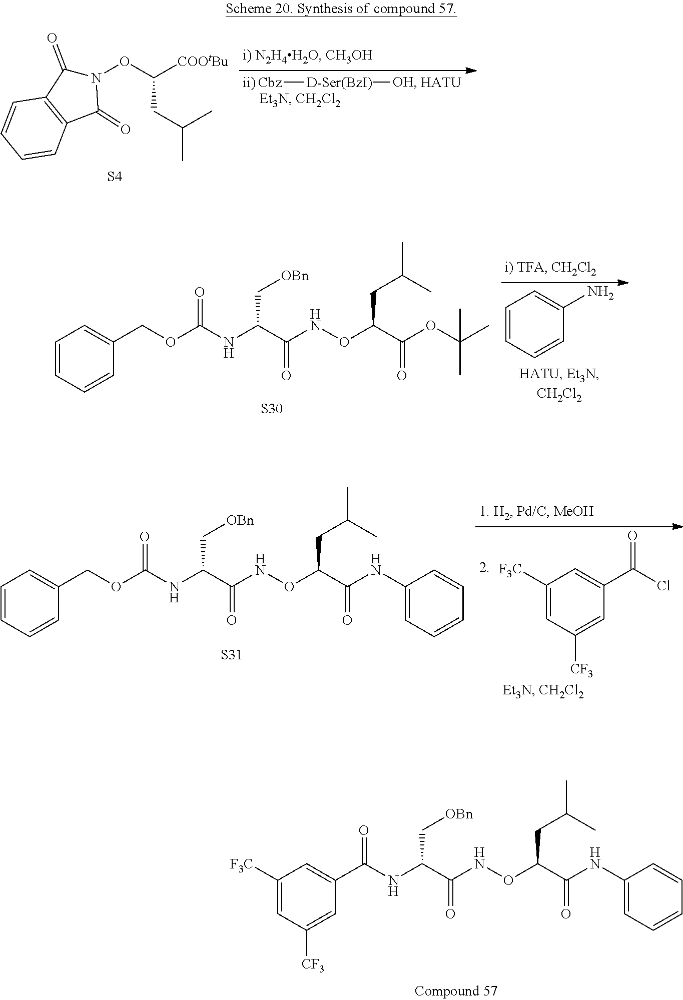

To starting material S4 (150 mg, 443 μmol) in CH₃OH solution (4 mL) was added N₂H₄·H₂O (67 mg, 1.33 mmol). After stirring for 1 hour at room temperature, the reaction mixture was concentrated under vacuo. The residue was dissolved in CH₂Cl₂ and filtered. The filtrated was concentrated to afford the free amine as a colorless oil, which was used in subsequent reaction without further purification. To a CH₂Cl₂ solution (4 mL) of Cbz-D-Ser(Bzl)-OH (146 mg, 443 μmol) were added trimethylamine (45 mg, 443 μmol) and HATU (168 mg, 443 μmol). After the mixture was stirred for 10 minutes, the free amine was added dropwise. The resulting reaction mixture was stirred overnight. The reaction mixture was washed with saturated NaHCO₃ solution and 0.1 M HCl aqueous solution subsequently. Then organic layer was dried over anhydrous Na₂SO₄ and concentrated under vacuo. The reaction mixture was purified by flash chromatography to afford colourless oil compound S30. (192 mg, 84% yield for 2 steps). R_{f}=0.3 (30% EtOAc in Hexane); ¹H NMR (500 MHz, CDCl₃) δ 9.40 (s, 1H), 7.40-7.47 (m, 10H), 5.71-5.51 (m, 1H), 5.10 (s, 2H), 4.55 (s, 2H), 4.35 (dd, J=9.9, 3.4 Hz, 1H), 4.33-4.24 (br, 1H), 4.66 (dd, J=10.6, 4.1 Hz, 1H), 3.86-3.72 (m, 1H), 3.48 (t, J=8.43 Hz, 1H), 2.03-1.91 (m, 1H), 1.75-1.66 (m, 1H), 1.54 (ddd, J=14.6, 8.8, 3.9 Hz, 1H), 1.44 (s, 9H), 1.01 (d, J=6.4 Hz, 3H), 0.96 (d, J=6.4 Hz, 3H); ¹³C NMR (126 MHz, CDCl₃) δ 171.3, 167.8, 156.0, 137.0, 136.1, 128.7, 128.4, 128.2, 128.1, 127.9, 82.6, 82.4, 73.7, 69.2, 67.3, 52.4, 39.9, 28.1, 24.8, 23.2, 21.8; HRMS (ESI) for C₂₈H₃₉N₂O₇ (M+H)⁺: calculated 515.2752, found 515.2773.

To a CH₂Cl₂ solution (2 mL) of compound S30 (192 mg, 373 μmol) was added trifluoroacetic acid (2 mL). The reaction mixture was stirred for 1 hour, then concentrated under vacuo and azeotroped with toluene 3 times to remove residual trifluoroacetic acid to afford free carboxylic acid, which was used in subsequent reaction without further purification. To a CH₂Cl₂ solution (4 mL) of free carboxylic acid were added HATU (142 mg, 373 μmol) and trimethylamine (38 mg, 373 μmol) subsequently. After the mixture was stirred for 15 minutes, aniline (38 mg, 37 μmol) was subsequently added and stirred for overnight. The reaction mixture was then concentrated under vacuo and purified by flash chromatography to afford white solid compound S31. R_{f}=0.4 (30% EtOAc in Hexane); ¹H NMR (500 MHz, CDCl₃) δ 10.17-10.08 (br, 1H), 9.98-9.87 (br, 1H), 7.67 (d, J=7.9 Hz, 2H), 7.37-7.21 (m, 10H), 7.18-7.11 (m, 2H), 7.08 (t, J=7.4 Hz, 2H), 5.72 (d, J=7.6 Hz 1H), 5.10-4.98 (m, 2H), 4.41-4.29 (m, 4H), 3.72-3.64 (m, 1H), 3.50 (dd, J=9.65, 6.38 Hz, 1H), 1.90-1.80 (m, 1H), 1.75 (t, J=6.74 Hz, 2H), 0.93 (d, J=6.5 Hz, 6H); ¹³C NMR (126 MHz, CDCl₃) δ 170.0, 169.7, 156.4, 138.0, 137.0, 135.8, 129.0, 128.7, 128.6, 128.5, 128.21, 128.16, 127.9, 124.4, 120.1, 86.2, 73.7, 69.2, 67.6, 52.7, 40.7, 24.9, 23.3, 21.8; HRMS (ESI) for C₃₀H₃₆N₃O₆ (M+H)⁺: calculated 534.2599, found 534.2630.

To a CH₃OH solution (5 mL) of compound S31 (193 mg, 362 μmol) was added Pd/C (20 mg). The reaction mixture was stirred for 1 hour under hydrogen gas, then filtered and concentrated under vacuo to afford free amine, which was used in subsequent reaction without further purification. To a CH₂Cl₂ solution (5 mL) of free amine were added 3,5-Bis(trifluoromethyl)benzoyl chloride (110 mg, 398 μmol) and trimethylamine (40 mg, 398 μmol) and stirred for overnight. The reaction mixture was then concentrated under vacuo and purified by flash chromatography to afford white solid compound 57. R_{f}=0.4 (30% EtOAc in Hexane); ¹H NMR (500 MHz, CD₃OD) δ 8.45 (s, 2H), 8.14 (s, 1H), 7.64 (d, J=7.9 Hz, 2H), 7.28 (t, J=7.7 Hz, 2H), 7.25-7.15 (m, 5H), 7.09 (t, J=7.4 Hz, 1H), 4.75 (t, J=6.0 Hz, 1H), 4.47-4.35 (m, 3H), 3.78 (d, J=6.2 Hz, 2H), 2.00-1.88 (m, 1H), 1.78 (ddd, J=14.6, 8.9, 5.0 Hz, 1H), 1.69 (ddd, J=14.6, 8.8, 4.0 Hz, 1H), 1.01 (d, J=6.6 Hz, 3H), 0.98 (d, J=6.6 Hz, 3H); ¹³C NMR (126 MHz, CD₃OD) δ 170.9, 170.5, 166.8, 139.1, 138.8, 137.3, 132.9 (q, J=33.7 Hz), 129.8, 129.43, 129.39, 128.85, 128.77, 126.3-126.1 (m due to C—F splitting), 125.6, 124.5 (q, J=273.0 Hz), 121.5, 86.6, 74.3, 70.0, 53.5, 42.0, 25.8, 23.6, 22.2; ¹⁹F NMR (471 MHz, CD₃OD) δ −64.2; HRMS (ESI) for C₃₁H₃₂F₆N₃O₅ (M+H)⁺: calculated 640.2241, found 640.2279.

The subject invention also concerns kits comprising in one or more containers of at least 1, 2, 3, 4, 5, 6, 7, 8, 9, or 10 compounds of the subject invention. A kit of the invention can also comprise one or more compounds, biological molecules, or drugs. In one embodiment, a kit of the invention comprises a compound of the subject invention. In certain embodiments, the subject invention further pertains to a kit comprising the compounds of the subject invention, compositions comprising the subject compounds and, optionally, other compounds, including, for example, antibiotics effective in treating gram-positive bacterial infections.

The invention further provides kits, including compounds of the subject invention and pharmaceutical formulations, packaged into suitable packaging material, optionally in combination with instructions for using the kit components, e.g., instructions for performing a method of the invention. In one embodiment, a kit includes an amount of compounds of the subject invention and instructions for administering the compounds of the subject invention to a subject in need of treatment on a label or packaging insert. In further embodiments, a kit includes an article of manufacture, for delivering the compounds of the subject invention into a subject locally, regionally or systemically, for example.

As used herein, the term “packaging material” refers to a physical structure housing the components of the kit. The packaging material can maintain the components sterilely and can be made of material commonly used for such purposes (e.g., paper, corrugated fiber, glass, plastic, foil, ampules, etc.). The label or packaging insert can include appropriate written instructions, for example, practicing a method of the invention, e.g., treating a bacteria infection, an assay for identifying a subject having a bacterial infection, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat a bacterial infection. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing the compounds of the subject invention. The kit can also include control components for assaying for the presence of bacterial cells, e.g., a control sample or a standard. Each component of the kit can be enclosed within an individual container or in a mixture and all of the various containers can be within single or multiple packages.

### Methods of Treating or Inhibiting a Bacterial Infection

In certain embodiment, at least 1, 2, 3, 4, 5, 6, 7, 8, 9, or 10 compounds of the subject invention are used for the treatment or inhibition of a gram-positive bacterial infection in a subject. In certain embodiment, the gram-positive bacterium is Staphylococcus aureus, Bacillus subtilis, Streptococcus pneumoniae, Listeria monocytogenes, Enterococcus faecalis or Mycobacterium smegmatis.

In certain embodiments, the subject compounds can be administered to a subject before the subject is infected with a gram-positive bacterium or to treat an existing bacterial infection. In certain embodiments, the compounds of the subject invention can be administered before, in combination with gentamicin, particularly an antibiotic that is an effective treatment of a gram-positive bacterial infection.

In certain embodiments, the compounds and/or compositions of subject invention can be used in methods of treating a bacterial infection comprising the step of administering therapeutically effect amounts of the compounds disclosed herein to the subject in need thereof. The gram-positive bacteria include, for example, Staphylococcus aureus, Bacillus subtilis, Streptococcus pneumoniae, Listeria monocytogenes, Enterococcus faecalis or Mycobacterium smegmatis.

Therapeutic or prophylactic application of the subject compounds and compositions containing the compounds thereof, can be accomplished by any suitable therapeutic or prophylactic method and technique presently or prospectively known to those skilled in the art. The compounds can be administered by any suitable route known in the art including, for example, oral, intramuscular, intraspinal, intracranial, nasal, rectal, parenteral, subcutaneous, or intravascular (e.g., intravenous) routes of administration. Administration of the compounds of the invention can be continuous or at distinct intervals as can be readily determined by a person skilled in the art.

In some embodiments, an amount of the compounds can be administered 1, 2, 3, 4, or times per day, for 1, 2, 3, 4, 5, 6, 7, or more days. Treatment can continue as needed, e.g., for several weeks. Optionally, the treatment regimen can include a loading dose, with one or more daily maintenance doses. For example, in some embodiments, an initial loading dose in the range of about 1 mg/kg to about 1000 mg/kg or about 10 mg/kg to about 100 mg/kg is administered every day for 1, 2, 3, 4, 5, 6, 7, 10, 14, 21, 28, 35, 42, 49, 56 or more days.

To provide for the administration of such dosages for the desired therapeutic treatment, pharmaceutical compositions of the invention will advantageously comprise between about 0.1% and 45%, and especially, 1% and 15% by weight of the total of one or more of the compounds based on the weight of the total composition including carrier or diluent.

In certain embodiments, the compounds have low toxicity towards mammalian cells, including, for example, NIH3T3 and no observable hemolysis activity. In certain embodiments, the compounds induce cell death of gram-positive bacteria over mammalian cells with up to 30-fold selectivity.

In certain embodiments, the compounds can eradicate gram-positive bacterial persister, reduce established biofilms of gram-positive bacteria, and inhibit the formation gram-positive biofilms. In certain embodiments, the compounds can suppress the secretion of the virulence protein of gram-positive bacteria. In certain embodiments, the compound can promote the hyperpolarization of bacterial membrane potential, calcium influx, acceleration of the bacteria autolysis and the disruption of cytoplasmic pH, in which the bacteria can burst due to accelerated autolysis. All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Compounds from Aminoxy Acids Library can Inhibit the Growth of the Gram-Positive Bacteria and has Great Selectivity on Bacteria Over Mammalian Cell NIH 3T3 and Human Red Blood Cell

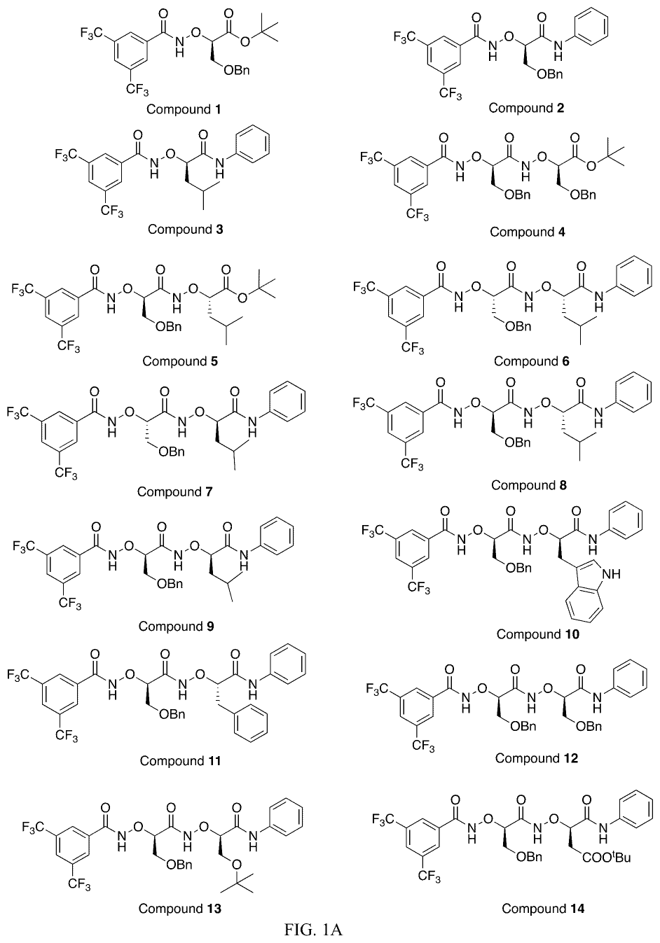

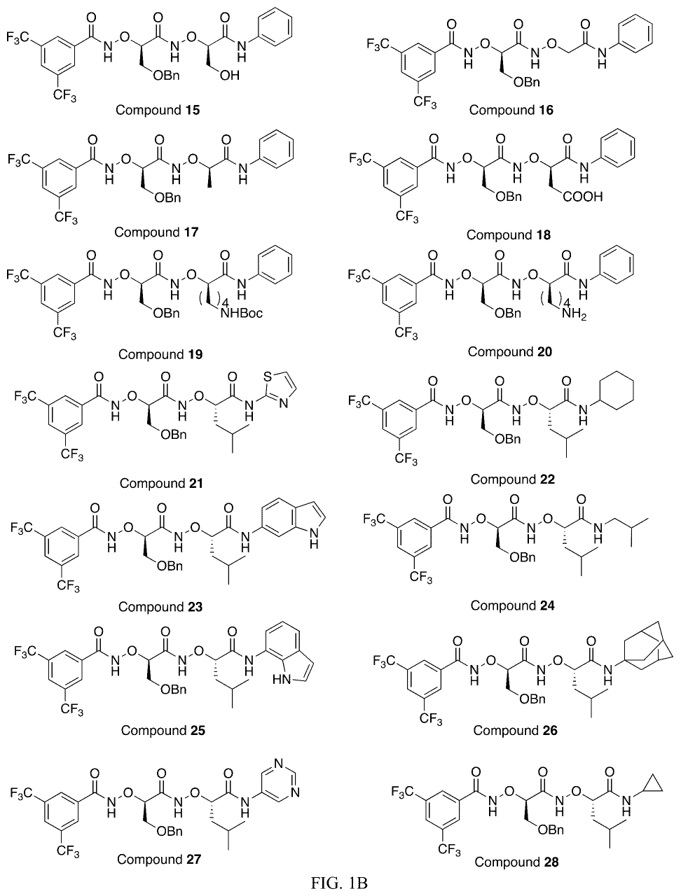

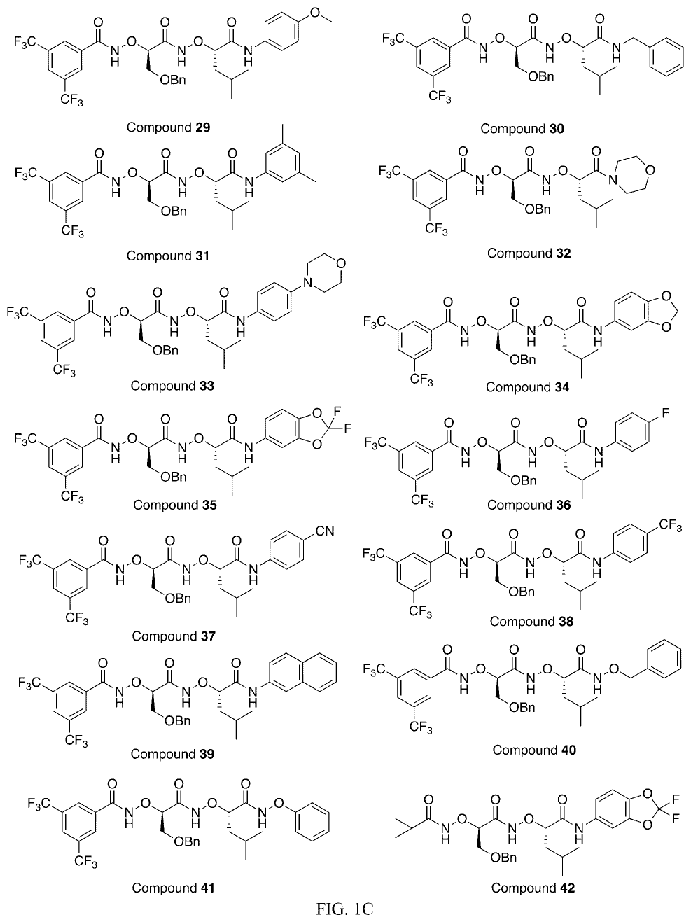

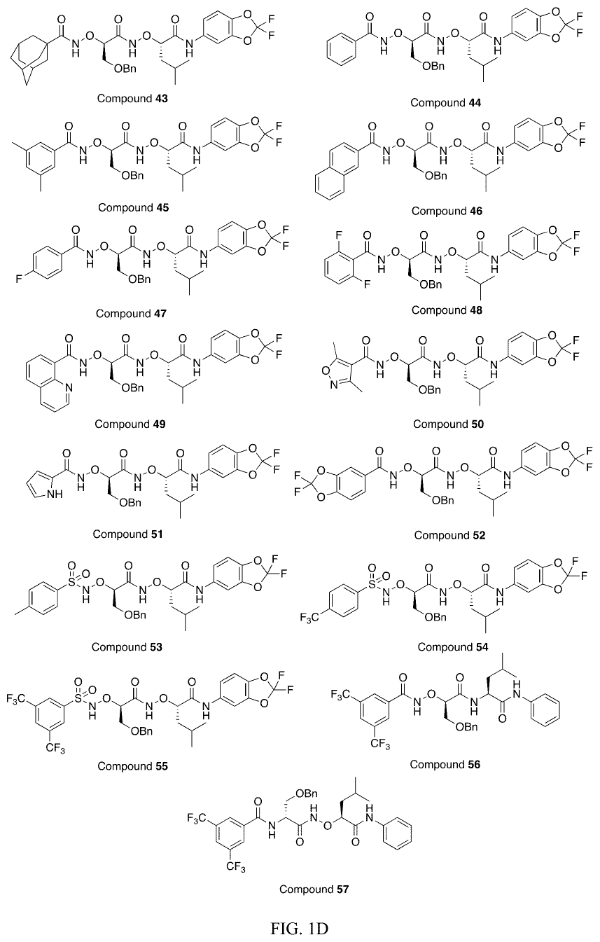

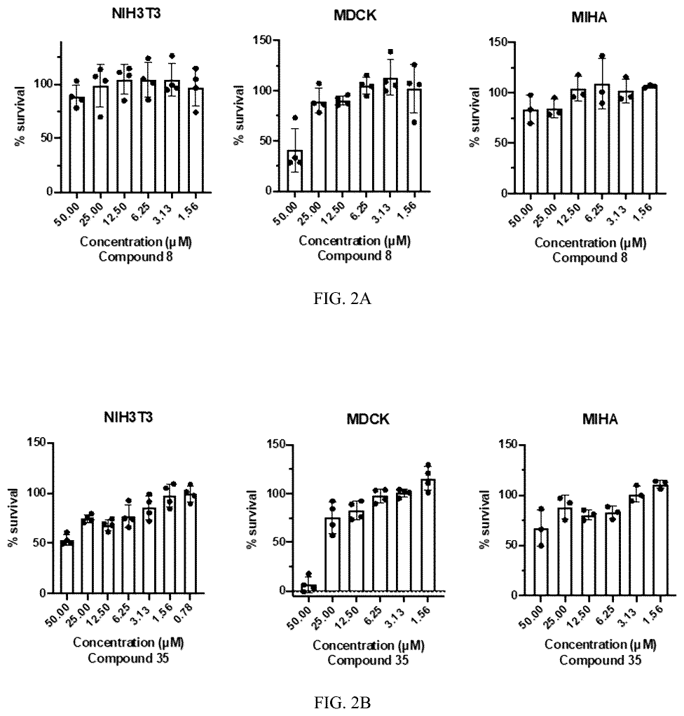

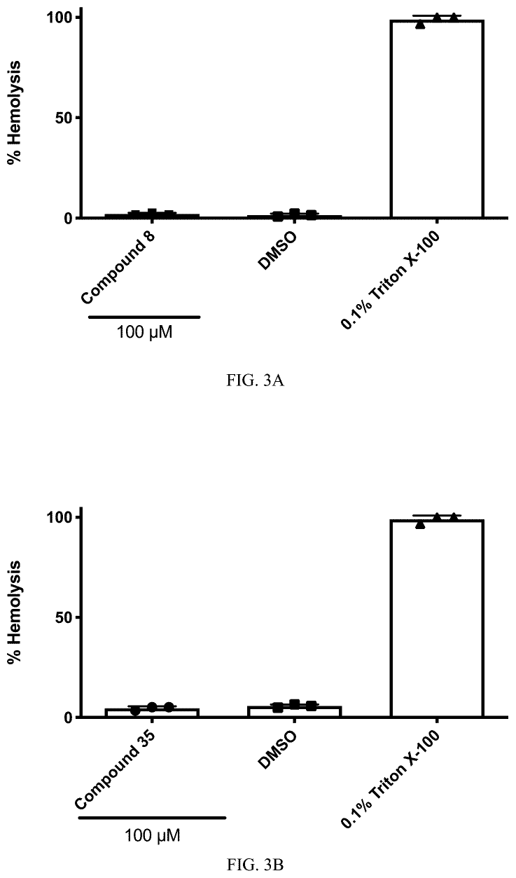

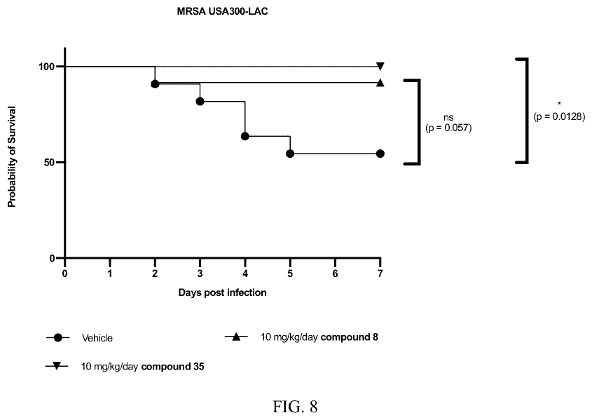

Aminoxy acids were synthesized by the standard peptide coupling procedures. Their structures are shown in FIGS. 1A-1D. Minimum inhibitory concentration was determined using CLSI broth micro-dilution method in 96-wells microtitre plates. Minimum inhibitory concentration is defined as the lowest concentration of the compound that inhibits visible growth of the bacteria. All experiments were performed three times. 24 compounds including aminoxy acid monomers and dipeptides have been identified to show toxicity against multidrug resistant Staphylococcus aureus Mu3 and summarized in Table 1. The minimum inhibitory concentration of compound 8 and compound 35 on other gram-positive bacteria was also evaluated and summarized in Table 2. These data clearly suggested that compounds from aminoxy acids library can inhibit the growth of the gram-positive bacteria. As shown in FIGS. 2A-2B, compound 8 and compound 35 showed minimal toxicity against mammalian cell NIH3T3, MDCK and MIHA at 16 folded MIC (50 μM and 12.5 μM respectively). As shown in FIGS. 3A-3B there are no observable hemolysis for compound 8 and compound 35 towards human red blood cell. As shown in FIG. 8, compound 8 and compound 35 can rescue the mice from the Staphylococcus aureus bacteremia infection. These data suggested compound 8 and compound 35 has excellent selectivity towards gram-positive bacteria over mammalian cell line.

TABLE 1
Minimum inhibitory concentration of compounds on
multidrug resistant Staphylococcus aureus Mu3

| | Minimum inhibitory concentration |
|---|---|
| | (μM) in MRSA Mu3 |
| Compound 1 (Formula I)) | 12.5 |
| Compound 2 (Formula II)) | 6.25 |
| Compound 3 (Formula III)) | 3.13 |
| Compound 4 (Formula IV)) | >12.5 |
| Compound 5 (Formula V)) | >12.5 |
| Compound 6 (Formula VI)) | 3.13 |
| Compound 7 (Formula VII)) | 3.13 |
| Compound 8 (Formula VIII)) | 3.13 |
| Compound 9 (Formula IX)) | 3.13 |
| Compound 10 (Formula X)) | 6.25 |
| Compound 11 (Formula XI)) | 3.13 |
| Compound 12 (Formula XII)) | 3.13 |
| Compound 13 (Formula XIII)) | 6.25 |
| Compound 14 (Formula XIV)) | 3.13 |
| Compound 15 (Formula XV)) | >12.5 |
| Compound 16 (Formula XVI)) | >12.5 |
| Compound 17 (Formula XVII)) | >12.5 |
| Compound 18 (Formula XVIII)) | >12.5 |
| Compound 19 (Formula XIX)) | 12.5 |
| Compound 20 (Formula XX)) | >12.5 |
| Compound 21 (Formula XXI)) | >12.5 |
| Compound 22 (Formula XXII)) | >12.5 |
| Compound 23 (Formula XXIII)) | >12.5 |
| Compound 24 (Formula XXIV)) | >12.5 |
| Compound 25 (Formula XXV)) | 6.25 |
| Compound 26 (Formula XXVI)) | 6.25 |
| Compound 27 (Formula XVII)) | >12.5 |
| Compound 28 (Formula XVIII)) | >12.5 |
| Compound 29 (Formula XXIX)) | 6.25 |
| Compound 30 (Formula XXX)) | >12.5 |
| Compound 31 (Formula XXXI)) | 3.13 |
| Compound 32 (Formula XXXII)) | >12.5 |
| Compound 33 (Formula XXXIII)) | >12.5 |
| Compound 34 (Formula XXXIV)) | 6.25 |
| Compound 35 (Formula XXXV)) | 0.781 |
| Compound 36 (Formula XXXVI)) | 3.13 |
| Compound 37 (Formula XXXVII)) | 6.25 |
| Compound 38 (Formula XXXVIII)) | 1.56 |
| Compound 39 (Formula XXXIX)) | 1.56 |
| Compound 40 (Formula XL)) | >12.5 |
| Compound 41 (Formula XLI)) | >12.5 |
| Compound 42 (Formula XLII)) | >12.5 |
| Compound 43 (Formula XLIII)) | >12.5 |
| Compound 44 (Formula XLIV)) | >12.5 |
| Compound 45 (Formula XLV)) | >12.5 |
| Compound 46 (Formula XLVI)) | >12.5 |
| Compound 47 (Formula XLVII)) | >12.5 |
| Compound 48 (Formula XLVIII)) | >12.5 |
| Compound 49 (Formula XLIX)) | >12.5 |
| Compound 50 (Formula L)) | >12.5 |
| Compound 51 (Formula LI)) | >12.5 |
| Compound 52 (Formula LII)) | 3.13 |
| Compound 53 (Formula XLIII)) | >12.5 |
| Compound 54 (Formula LIV)) | >12.5 |
| Compound 55 (Formula LV)) | >12.5 |
| Compound 56 (Formula LVI)) | 6.25 |
| Compound 57 (Formula LVII)) | >12.5 |

TABLE 2
Minimum inhibitory concentration of compound 8 and
compound 35 on other gram-positive bacteria.

Minimum inhibitory concentration (μM)

| Bacteria strain | Compound 35 | Compound 8 |
|---|---|---|
| MSSA ATCC 29213 | 1.56 | 3.13 |
| MSSA ATCC 35556 | 0.78 | 3.13 |
| MSSA ATCC 6538 | 1.56 | 3.13 |
| MRSA Mu3 | 0.78 | 3.13 |
| MRSA-USA300 | 3.13 | 3.13 |
| MRSA-USA300LAC | 1.56 | 3.13 |
| B. subtilis WT168 | 1.56 | 3.13 |
| L. monocytogenes | 0.78 | 6.25 |
| clinical strain | | |
| E. faecalis | 6.25 | 3.13 |
| clinical strain | | |
| M. smegmatis mc²155 | 1.56 | 12.5 |

MSSA = Methicillin susceptible Staphylococcus aureus;
MRSA = Methicillin resistant Staphylococcus aureus;
N.D. = Not determined.

### Example 2—Compound 8 and Compound 35 can Eradicate Staphylococcus aureus Persisters, Reduce Established Biofilm, Inhibit Biofilm Formation and Suppress Virulence Proteins Secretion

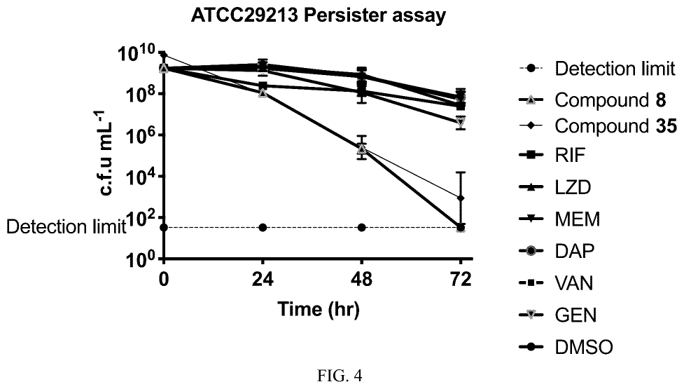

Persisters are the phenotypic variants of the normal bacteria with reduced metabolism. They highly tolerate to the conventional bacterial. Persisters was enriched by the addition of 100-fold minimum inhibitory concentration of bactericidal antibiotics ciprofloxacin to the stationary phase bacteria. After 24 hours' culture, ciprofloxacin was removed. Persisters were treated with compound 8, compound 35 or different conventional antibiotics. As shown in FIG. 4, compound 8 and compound 35 can eradicate Staphylococcus aureus persister while other conventional antibiotics have no effect.

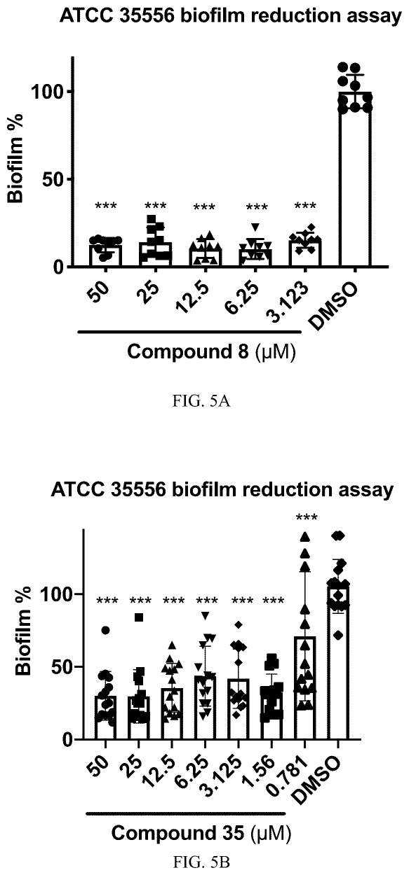

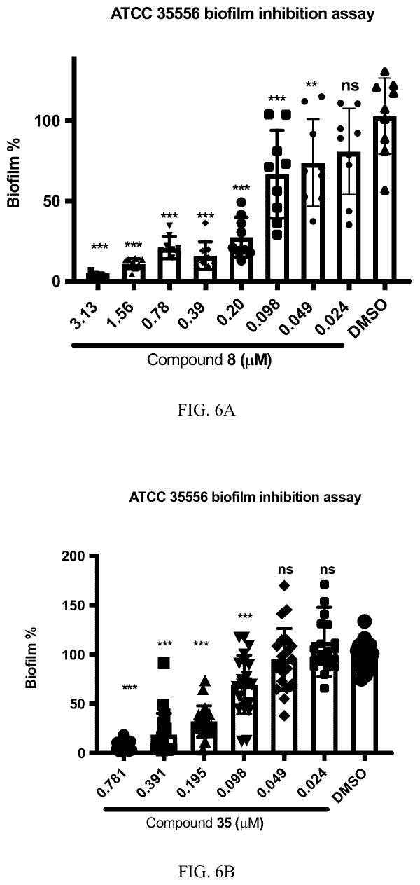

Biofilm is the bacterial polymeric network which resists to most of the antibiotics. Results in FIGS. 5A-5B have shown that compound 8 and compound 35 can significantly reduce the pre-established Staphylococcus aureus biofilm. Apart from biofilm reduction, compound 8 and compound 35 can also inhibit the formation of Staphylococcus aureus biofilm as shown in FIGS. 6A-6B.

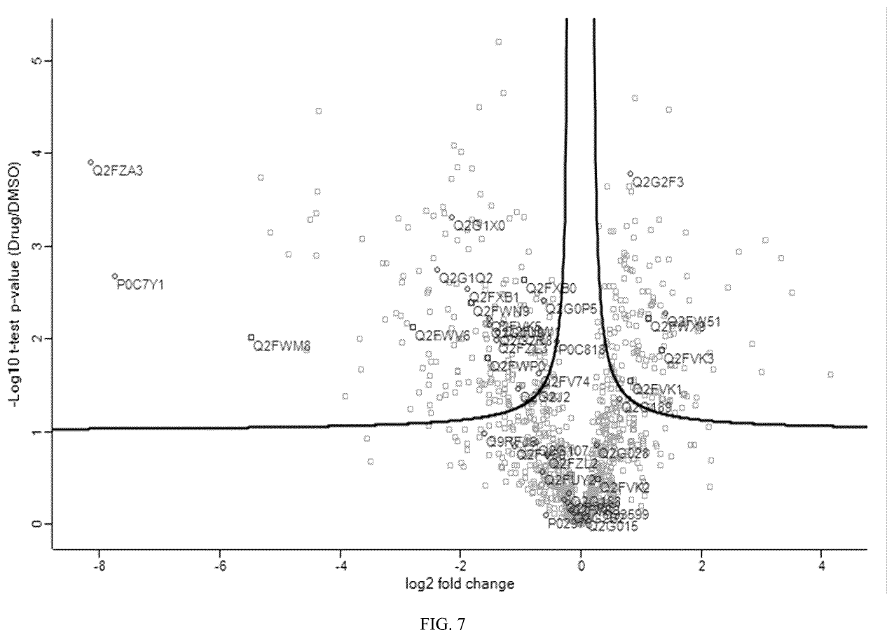

Staphylococcus aureus is also well known for its arsenal of virulence proteins that can lead to hemolysis and severe organs failure. As shown in FIG. 7, the secretome analysis revealed the decrease of the amount of virulence proteins in supernatant after the addition of sub-inhibitory concentration of compound 8, indicating that compound 8 can suppress virulence protein secretion.

### 5.3 Compound 8 can Alter Bacteria Membrane Potential, Acidify Cytoplasm, Induce Calcium Influx and Accelerate Autolysis.

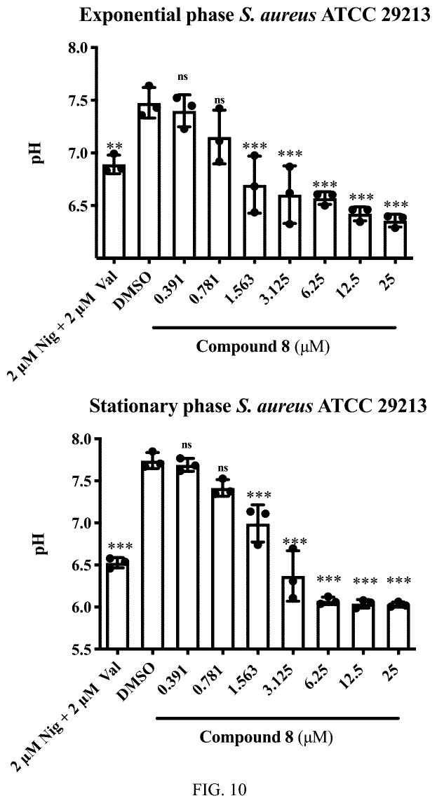

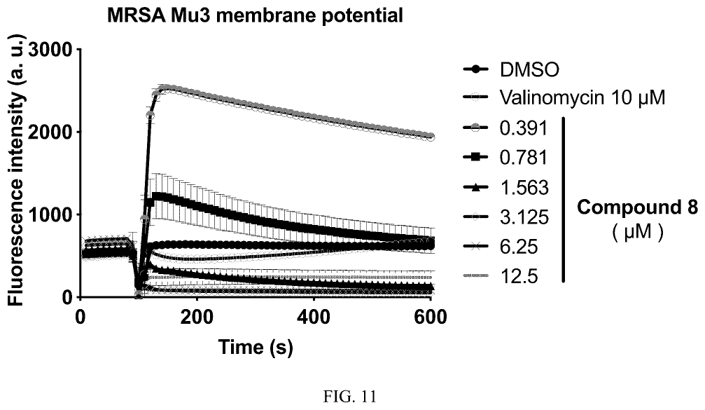

Compounds 8 can lead to bacterial cytoplasm acidification. As shown in FIG. 10, compounds 8 lead to cytoplasm acidification in a concentration dependent manner. This also suggested the pH elevation of cell wall since the proton was transported into cytoplasm form cell wall. The effect of compounds 8 on bacterial membrane potential was also investigated on Staphylococcus aureus. As shown in FIG. 11, compound 8 can depolarize Staphylococcus aureus cell membrane under sub-inhibitory concentration and hyperpolarize Staphylococcus aureus cell membrane under inhibitory concentration. Membrane potential was monitored by DiSc₃(5) over time (s).

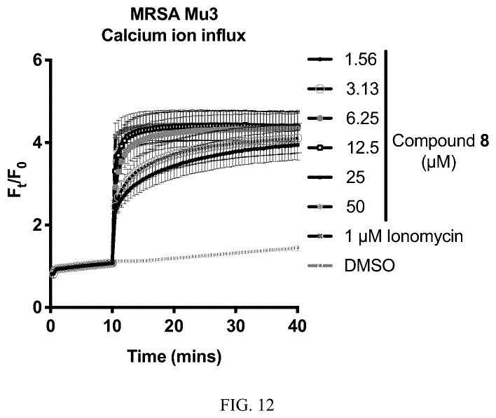

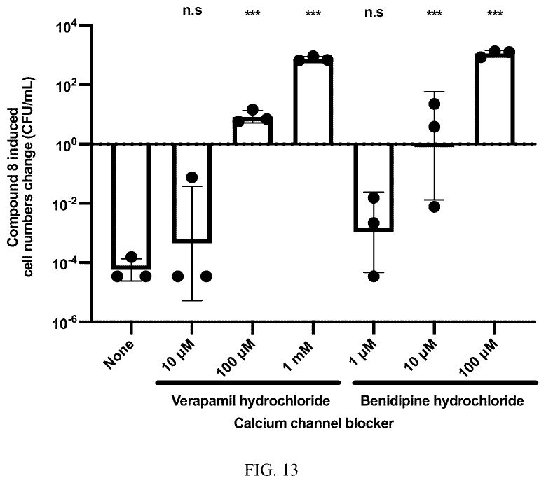

Activation or closure of calcium channel is often controlled by membrane potential and calcium influx is known as programmed cell death signal in mammalian cells. As shown in FIG. 12, compound 8 can induce calcium influx into Staphylococcus aureus cytoplasm. Cytoplasmic calcium ion content was monitored Fluo-4-am over time (s). Such calcium influx is lethal to the bacteria. As shown in FIG. 13, calcium channel blockers can antagonize the antibacterial effect of compound 8 on Staphylococcus aureus.

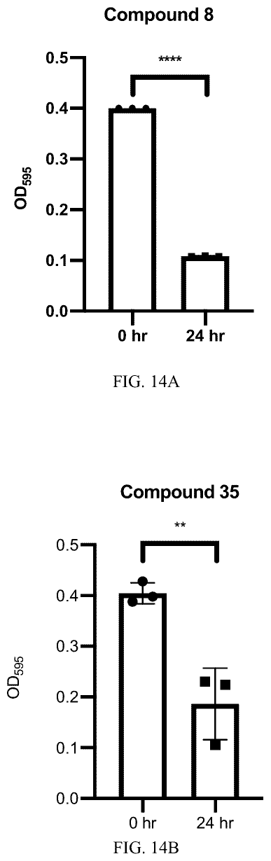

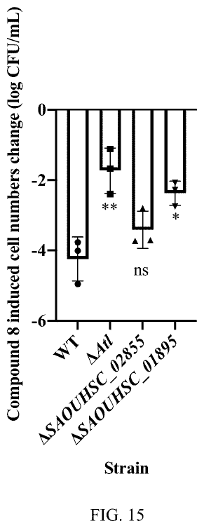

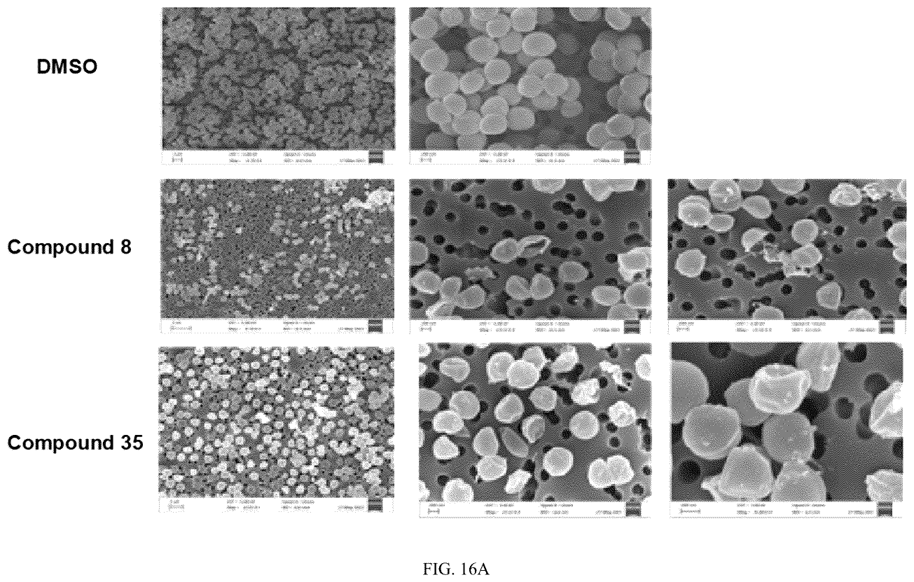

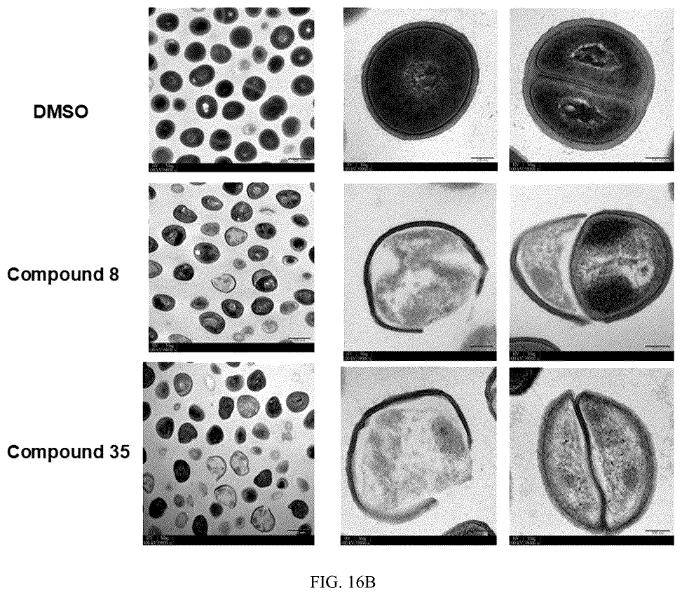

Calcium influx with membrane potential and pH alternation lead to dysregulation of the bacterial autolysis. As shown in the FIGS. 14A-14B, addition of compound 8 and compound 35 lead to the reduction of the optical density of the culture of Staphylococcus aureus, indicating accelerated autolysis. Moreover, compounds 8 had diminished bactericidal effect on autolysin deletion mutants of Staphylococcus aureus as shown in FIG. 15. Accelerated autolysis degrades bacterial cell wall and lead to osmotic cell burst. FIGS. 16A-16B demonstrated treatment of compounds 8 induced bacterial cell burst while the bacterial cell wall treated by DMSO control remained intact.

### Example 3—57 Compounds were Tested their Ability to Inhibit the Growth of Multi-Drug Resistant Staphylococcus aureus Mu3

### Glycerol Stocks of Bacteria

Bacteria isolate was cultured overnight in Luria broth (LB) or Brain heart infusion (BHI) with appropriate antibiotics if necessary. 200 μL of 25% glycerol were mixed with 800 μL overnight bacteria culture with a vortex in a screw-cap tube. Glycerol stocks were stored at −80° C.

### Bacteria Culture

Bacteria were streaked from the glycerol stock into BHI agar and were incubated overnight. Single colony was picked for the overnight culture in cation-adjusted Mueller Hinton broth (CAMH), LB or BHI. The overnight culture was sub-cultured and regrew to exponential phase for the experiment.

### Minimum Inhibitory Concentration (MIC) Determination by Broth Dilution

MIC was determined by CLSI broth micro-dilution method in 96-wells microtitre plates. For compounds dissolved in water, 100 μL of 2× testing concentration was added to the first well of each column, followed by two-fold serial dilution for successive wells with 50 μL medium. For chemical compounds dissolved in DMSO, the compounds were two-fold serial diluted by DMSO in a DMSO-compatible 96-well plate. 1 μL of compound in DMSO was transferred into 96-wells microtitre plate containing 50 μL medium to give 2× testing concentration. An overnight culture of bacteria was grown into exponential phase. The bacteria culture was diluted to 1×10⁶ CFU/mL and 50 μL of diluted culture were added into each well. The test plates were kept in a 37° C. incubator overnight. Optical density was recorded by DTX 880 multimode detector (Beckman Coulter, California, US). MIC is defined as the lowest concentration of the compound that inhibits visible growth of bacteria. Biological replicates were performed. CAMH was used for bacteria culture and MIC test except S. pneumoniae, L. monocytogenes, E. faecalis and M. smegmatis for which BHI was used as culture medium

### Results

After screening a small library of aminoxy acid based compounds, 24 molecules can inhibit bacterial growth, with the minimum inhibitory concentration lower than 12.5 μM (Table 1). Among them, compound 35 showed lowest minimum inhibitory concentration (0.781 μM).

### Example 4—Compound 8 and Compound 35 have Low Cytotoxicity Towards Mammalian Cell NIH3T3 and Red Blood Cell

### Mammalian Cell Culture

NIH3T3 cells were cultured in DMEM supplemented with 10% fetal bovine serum (FBS), 100 U/ml penicillin and 0.1 mg/ml streptomycin. Cells were grown in treated culture flasks at 37° C. in a 5% CO₂ humidified incubator and passaged every 2-3 days.

### Mammalian Cell Cytotoxicity Assay

In the treated culture dish, cells were washed by PBS and then trypsinized. DMEM was added to quench the reaction. Cells were centrifuged and resuspended into fresh DMEM. Cells were then seeded in 96-well microtitre plate (100 μL, 5000 cells per well) for 24 hours before addition of compounds. The compounds were two-fold serial diluted by DMSO in a DMSO-compatible 96-well plate. 1 μL of compound in DMSO is transferred into the 96-well microtitre preparation plate containing 100 μL DMEM medium. The medium in culture plate was removed and compounds in preparation plate were transferred to culture plate. Equal volume of DMSO was used as negative control.

After 24 hours of incubation of cells, cell viability was measured by MTT assay. In MTT assay, 20 μL 5 mg/mL MTT solution in PBS was added to cell medium and cultured for 3 hours. The medium was then removed and 50 μL DMSO was added and cultured for 10 minutes. Absorbance at 595 nm was measured spectrophotometrically (Beckman Coulter DTX 880 Multimode Detector) and normalized to DMSO group. Biological replicates were performed.

### Hemolysis Assay

Citrate-phosphate-dextrose (CPD) human whole blood was obtained from Hong Kong Red Cross. Whole blood was centrifuged in 1000 rpm for 10 minutes to remove the upper plasma layer. The remaining portion was washed by 1×PBS 3 times or until the upper PBS layer was transparent. The upper PBS layer was removed and the lower blood cell layer was diluted into 2% in PBS. 200 μL 2% blood in PBS was then added to round-bottom 96-well plate. Compounds were subsequently added to 96-well plate on 1% DMSO concentration. After incubation in 37° C. for 2 hours, the round-bottom 96-well plate was centrifuged in 1000 rpm for 10 minutes. 50 μL supernatant was transferred to a flat-bottom 96-well plate. Absorbance at 405 nm was then measured by Beckman Coulter DTX 880 Multimode Detector. Biological replicates were performed.

### Results

As shown in FIGS. 2A-2B, compound 8 and compound 35 had low toxicity towards mammalian cells NIH3T3 with IC₅₀ more than 50 μM. As shown in FIGS. 3A-3B, compound 8 and compound 35 didn't have observable hemolytic activity on human blood cell even at the highest concentration 100 μM. Further investigations were focused on compound 8 and compound 35 due to their superior selectivity on bacteria

### Example 5—Compound 8 and Compound 35 can Eradicate Staphylococcus aureus Persister

S. aureus ATCC 29213 cells were sub-cultured from an overnight culture in CAMH 1:100 for 24 hours at 37° C. with 250 rpm shaking speed. Persisters were prepared by addition of ciprofloxacin hydrochloride (25 μg/mL, 100×MIC in Staphylococcus aureus ATCC 29213) and cultured for 24 hours at 37° C. with 250 rpm shaking speed. Persisters were washed by PBS (12,000×g, 3 minutes) and resuspended in PBS+1% CAMH medium (same volume as original culture). 1 mL portions were aliquoted into fresh culture tubes, and different antibiotics or DMSO (1% maximum concentration) were added, followed by incubation at 37° C. with 250 rpm shaking speed. At different time point, 20 μL bacteria culture was serially diluted and 10 μL was spot plated on the MHA. After incubating the plates overnight at 37° C., colonies were enumerated as the number of colony forming unit. The detection limit was 33 CFU/mL. The experiments were conducted in biological triplicates. Data represented the mean±S.D. of biological triplicates.

### Results:

Bacterial persister is known to highly tolerant to the conventional bacteria. Moreover, bacterial persister can be enriched via the addition of the antibiotics. As shown in FIG. 4, after the treatment of ciprofloxacin, the conventional antibiotics has no bactericidal effect on Staphylococcus aureus bacteria. However, both compound 8 and compound 35 can eradicate the Staphylococcus aureus persister.

### Example 6—Compound 8 and Compound 35 can Reduced Staphylococcus aureus Biofilm

Briefly, overnight cultures of Staphylococcus aureus ATCC 35556 in TSB were diluted 1:100 and regrow for 2.5 hours at 37° C., 250 rpm. The bacteria were diluted 1:100 into fresh TSB and 200 μL bacteria in TSB was added to each well of a flat-bottom 96-well plate (200 μL/well). The plate was incubated at 37° C. for 24 hours to establish biofilms. After 24 hours, the supernatant was removed and washed by PBS (200 μL) 2 times. Premixed solution of TSB and compound was prepared in a separate 96-well-plate (200 μL/well; two-folded serial dilutions; final DMSO concentration of maximum 1%). 200 μL compound in TSB was transferred to the plates with established biofilms. Plates were incubated at 37° C. for further 24 hours, then the medium from each well were removed and biofilms were washed with PBS (200 μL) 2 times to remove planktonic cells. Biofilms were dried at 37° C. for 30 minutes. 1% Crystal violet (25% ethanol in H₂O) was diluted 1:5 in PBS and 50 μL was added to each well, followed by incubation at room temperature for 10 minutes. After removal of excess crystal-violet solution, biofilms were washed with PBS (3×200 μL) and the remaining coloring agent was dissolved in 33% acetic acid in H₂O (200 μL) with 20 minutes' incubation at room temperature. Following a 1:4 dilution step (33% acetic acid), the optical density was measured at 595 nm by Beckman Coulter DTX 880 Multimode Detector. Data represented the mean±S.D. of biological triplicates. Statistical analysis was performed by ANOVA with Dunnett's post-test to calculate p-values adjusted for multiple comparisons with DMSO as control condition (*p<0.05; **p<0.01; ***p<0.001).

### Results:

It is well known that Staphylococcus aureus biofilm is highly tolerated to the conventional biofilm. The bactericidal effect of compound 8 and compound 35 on established biofilm was evaluated. As shown in FIGS. 5A-5B is a bar graph showing the established Staphylococcus aureus biofilm treating with compound 8 and compound 35 in a concentration dependent manner are significantly reduced.

### Example 7—Compound 8 and Compound 35 can Inhibit Staphylococcus aureus Biofilm Formation

Briefly, overnight cultures of Staphylococcus aureus ATCC 35556 in Tryptic Soy Broth (TSB) were sub-cultured from an overnight culture 1:100 for 2.5 hours. The bacteria were diluted 1:100 into fresh TSB and 200 μL bacteria in TSB was added to each well of a flat-bottomed 96-well plate (200 μL/well). Compounds (two-fold serial dilutions; final DMSO concentration of maximum 1%) were added. Plates were incubated at 37° C. for 24 hours. After 24 hours, the supernatant was removed and washed by PBS (200 μL) 2 times. Biofilms were dried at 37° C. for 30 minutes. 1% Crystal violet (25% ethanol in H₂O) was diluted 1:5 in PBS and 200 μL was added to each well, followed by incubation at room temperature for 10 minutes. After removal of excess crystal-violet solution, biofilms were washed with PBS (3×200 μL) and the remaining coloring agent was dissolved in 33% acetic acid in H₂O (200 μL) with 20 minutes' incubation at room temperature. Following a 1:4 dilution step (33% acetic acid), optical density was measured at 595 nm by Beckman Coulter DTX 880 Multimode Detector. Data represented the mean±S.D. of biological triplicates. Statistical analysis was performed by ANOVA with Dunnett's post-test to calculate p-values adjusted for multiple comparisons with DMSO as control condition (*p<0.05; **p<0.01; ***p<0.001).

### Results:

Compounds that prevent the formation of biofilm can be a good coating material to make surgery device. As shown in FIGS. 6A-6B, compound 8 and compound 35 can significantly inhibited the formation of the Staphylococcus aureus biofilm even under sub-inhibitory concentration.

### Example 8—Compound 8 can Suppress Staphylococcus aureus Virulence Proteins Production

### Secretome Analysis

S. aureus ATCC 29213 cells were sub-cultured from an overnight culture to OD595 0.1 and then cultured for 5 hours at 37° C., 250 rpm. Cells were concentrated by centrifugation (3,000×g, 4° C., 15 minutes), washed with 1×PBS and resuspended in CAMH to give OD595 1.5. 10 mL bacteria in CAMH were incubated with compound 8 (0.5×MIC; 1.56 μM) or DMSO control for 1.5 hours at 37° C., 250 rpm. Cells were then concentrated by centrifugation (3,000×g, 15 minutes and 6,000×g, 5 minutes). The supernatant was filtered (0.2 μm) and then concentrated by Amicon Ultra 10 KDa filter. Protein concentration was quantified by Bradford assay.

### Protein Digestion

Proteins were reduced by TCEP (10 mM; 37° C., 200 rpm) for 1 hour and then alkylated by iodoacetamide (12.5 mM; 25° C., 200 rpm) for 30 minutes. To quench the reaction, dithiothreitol (12.5 mM; 25° C., 200 rpm) was added for 30 minutes. Endoprotinase LysC was subsequently added (Enzyme: protein ratio 1:200; 25° C., 700 rpm) for 2 hours. The samples were diluted 1:5 by 50 mM ammonium bicarbonate buffer and further digested by trypsin (Enzyme: protein ratio 1:20; 37° C.) for 16 hours. The reaction was quenched by the addition of formic acid until pH reached 2-3. Samples were then submitted to the HKU proteomics and metabolomics core facility for analysis.

### Label Free Protein Quantification

The peptides were desalted using C18 StageTips. Eluted peptides were analyzed with nanoelute UPLC was coupled to Bruker TimsTOF Pro mass spectrometer. The peptide mixture (500 ng) was loaded onto an Aurora C18 UPLC column (75 μm internal diameter×25 cm length×1.6 μm particle size (IonOpticks, Australia)). Chromatographic separation was carried out using a linear gradient of 2-37% of buffer B (0.1% FA in ACN) at a flow rate of 300 nL/min over 15 min. MS and MS/MS data were collected over a m/z range of 100 to 1700. During MS/MS data collection, each TIMS cycle was 1.1 second and included 1 MS+an average of 10 PASEF MS/MS scans.

### Data Analysis of Secretome

The high resolution, high mass accuracy MS data obtained were processed using Maxquant version 1.6.0.1, wherein data were searched using the Andromeda algorithm against Staphylococcus aureus NCTC8325 UniProt FASTA database (September 2020) containing 2889 proteins, using settings as below: oxidized methionine (M), and acetylation (Protein N-terminus) were selected as dynamic modifications, and carbamidomethyl (C) as fixed modifications with minimum peptide length of 7 amino acids was enabled. Confident proteins were identified using a target-decoy approach with a reversed database, strict false-discovery rate of 1% at peptide and PSM level. Proteins identified from both conditions were quantified using the peptide LFQ intensities and their ratio obtained were used for label free quantitation to calculate the fold change. Data visualization and statistical data analysis were performed by Perseus software version 1.6.0.2. Results: Staphylococcus aureus secret large amount of virulence proteins that counter human immune system and lead to organ failure. As shown in FIG. 7, the volcano plot showing compound 8 can reduce the secretion of the virulence proteins of Staphylococcus aureus.

### Example 9—Compound 8 and Compound 35 can Rescue Mice from Staphylococcus aureus Bacteremia Infection Model

Staphylococcus aureus strain USA300-LAC were cultured to the early exponential phase of growth, washed twice with sterile phosphate-buffered saline solution (PBS), and resuspended in PBS at 3×10⁷ CFU/200 μL for lethal bacteremia model. The female Balb/c mice, 6-8 weeks, were infected intravenously (i.v.) through the tail vein with Staphylococcus aureus and randomized into three groups (N=11 for vehicle; N=12 for compound 8 and compound 35 respectively). Six hour after infection, mice received intraperitoneal injections of compound 8 or compound 35 (10 mg/kg/day) or an injection of buffer (PBS with 2.5% DMSO and 2% Tween-80) as the control. The compound and injection buffer treatments were performed twice per day at 12-h intervals. The challenged mice were monitored for 7 days, at which point all the remaining mice were sacrificed. Statistical analysis was performed with a log rank test (Mantel Cox test). (*p<0.05).

### Results:

In the in vivo Staphylococcus aureus bacteremia infection model, compound 8 and compound 35 (10 mg/kg/day) can rescue mice from Staphylococcus aureus bacteremia infection model as shown in FIG. 8.

### Example 10—Compound 8 and Compound 35 can Reduce the Bacterial Loads in Different Organs of Mice Infected by Staphylococcus aureus

### Materials and Methods:

Staphylococcus aureus strain USA300-LAC were cultured to the early exponential phase of growth, washed twice with sterile phosphate-buffered saline solution (PBS), and resuspended in PBS at 5×10⁶ CFU/200 μL for non-lethal bacteremia model. The female Balb/c mice, 6-8 weeks, were infected intravenously (i.v.) through the tail vein with Staphylococcus aureus and randomized into three groups (N=14 for vehicle; N=12 for compound 8 and compound 35 respectively). One hour after infection, mice received intraperitoneal injections of compound 8 or compound 35 (10 mg/kg/day) or an injection of buffer (PBS with 2.5% DMSO and 2% Tween-80) as the control. The compound and injection buffer treatments were performed twice per day at 12-h intervals. The challenged mice were monitored for 4 days, at which point all the remaining mice were sacrificed. The CFU of S. aureus were determined in heart, liver, kidney and lung by plating in series dilution. Grubb's test was performed to test for outliers. Statistical analysis was performed with two-sided paired t test. (*p<0.05).

### Results:

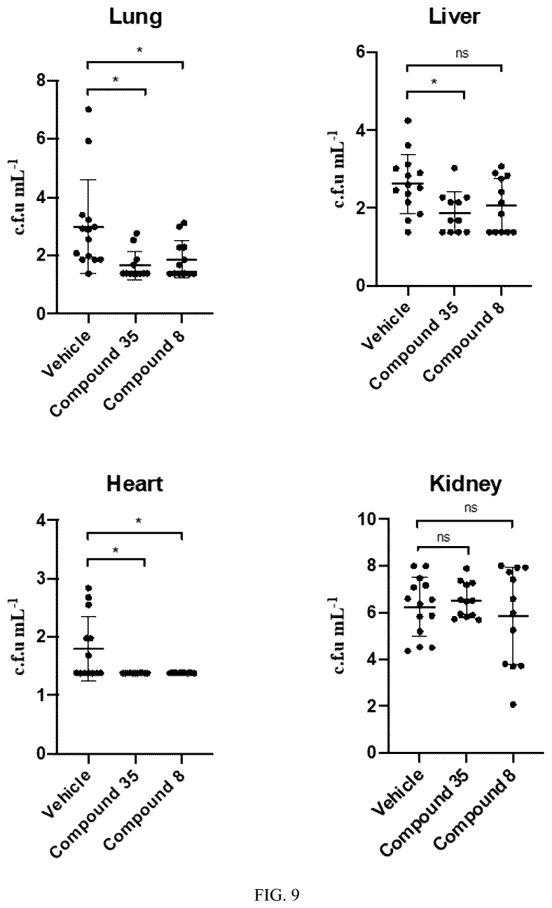

In the in vivo Staphylococcus aureus bacteremia infection model, compound 8 (10 mg/kg/day) can reduce the bacteria load in heart and lung, and compound 30 (10 mg/kg/day) can reduce the bacteria load in liver, heart and lung as shown in FIG. 9.

### Example 11—Compound 8 can Acidify Staphylococcus aureus Cytoplasm

Construction of pHluorinsarA Plasmid

To construct pHluorinsarA plasmid, pGLsarA plasmid vector was PCR amplified using primers pGL-f (CTGCAGGCATGCAAG; SEQ ID NO: 1) and pGL-r (GTTTAAAACCTCCCTAGTCGACG: SEQ ID NO: 2). Codon optimization of pHluorin for Staphylococcus aureus was performed and the gene was synthesized with the vector PUC57 by GenScript (Hong Kong) Limited based on the GenBank sequence (AF058694.2). pHluorin was PCR amplified using primers pHluorin-f (ATGTCAAAAGGTGAAGAATTATTTACAGGTGTTGTTCCTATTTTA; SEQ ID NO: 3) and pHluorin-r (TTATTTATATAATTCATCCATACCATGTGTAATACCTGC: SEQ ID NO: 4) followed by the addition of the overlapped region with pGL backbone using primers pHluorin-f2 (ACTAGGGAGGTTTTAAACATGTCAAAAGGTGAAGAATTATTTACAGGTGTTGTTC: SEQ ID NO: 5) and pHluorin-r2 (CTTGCATGCCTGCAGTTATTTATATAATTCATCCATACCATGTGTAATACCTGCTGC: SEQ ID NO: 6). The resulting PCR fragment was ligated to pGL backbone using Vazyme ClonExpress®II One Step Cloning Kit and transformed to E. coli top 10 competent cells. The construction was verified by colony PCR and sequencing using primers (RKC0482 GAAAGGGGGATGTGCTG: SEQ ID NO: 7) and pHluorin-r. Successful clone was then subsequently transformed into Staphylococcus aureus RN4200 and Staphylococcus aureus ATCC 29213.

Calibration of Fluorescence of pHluorin Response to Staphylococcus aureus Cytoplasmic pH

A pHlourin standard curve correlating pH and fluorescence ratio was constructed. Briefly, the emission at 510 nm was monitored with excitation wavelengths at 410 nm and 470 nm simultaneously following previously described protocol¹⁸ Overnight culture Staphylococcus aureus ATCC 29213 was sub-cultured 1:100 in fresh CAMH medium and regrown to exponential phase. The cells were then resuspended to OD₅₉₅ 1.0 in PBS of different pH. 2 μM valinomycin and 2 μM sodium nigericin were added in different pH buffer for 10 minutes to equilibrate extracellular and intracellular pH. Fluorescence ratio (Excitation wavelength 410 nm/470 nm; emission wavelength 510 nm) was then measured in 1 mL cuvette using Hitachi F-4500 Florescence Spectrometer and plotted against the pH of the buffer.

Measurement of Staphylococcus aureus Cytoplasmic pH Using pHluorinsarA Plasmid

An overnight culture of Staphylococcus aureus ATCC 29213 was sub-cultured 1:100 in fresh CAMH medium and regrown to exponential phase for bacterial cytoplasmic pH measurement. An overnight culture of Staphylococcus aureus ATCC 29213 was used directly for stationary phase bacterial cytoplasmic pH measurement. The bacteria were then resuspended to OD 1.0 in PBS and incubated with different concentration of compound 8 (1% DMSO), 2 μM valinomycin and 2 μM sodium nigericin as positive control or 1% DMSO as negative control for 5 minutes. Fluorescence ratio (410 nm/470 nm) of the bacteria were measured in 1 mL cuvette using Hitachi F-4500 Florescence Spectrometer.

### Results:

As shown in FIG. 10, compound 8 can acidify Staphylococcus aureus cytoplasm. Cytoplasm pH was monitored by pH sensitive fluorescent protein pHluorinsarA over time (s).

### Example 12—Compounds 8 can Depolarize Staphylococcus aureus Cell Membrane Under Sub-Inhibitory Concentration and Hyperpolarize Staphylococcus aureus Cell Membrane Under Inhibitory Concentration

Typically, an overnight culture of MRSA Mu3 was sub-cultured 1:100 in fresh CAMH and regrown to exponential phase. The bacteria were harvested (3,000×g, 5 min), washed, and re-suspended in a solution with 10 mM HEPEs buffer, 5 mM glucose and 100 mM KCl or 100 mM NaCl (pH 7.2). The bacteria were diluted to OD₅₉₅ 0.3 with the same buffer. DiSC₃(5) (excitation wavelength: 622 nm; emission wavelength: 670 nm) was added into bacteria solution with a final concentration 0.4 μM. Bacteria were incubated in darkness for 20 minutes or until fluorescence signal become stable. Fluorescence signal was recorded using Hitachi F-4500 Florescence Spectrometer. In a 1 mL cuvette, 594 μL bacteria incubated with DiSC₃(5) were added and the fluorescence was monitored for 100 s. At t=100 s, 6 μL compound in DMSO was added into the cuvette. The fluorescence was monitored for further 500 s. 10 μM valinomycin was used as the positive control.

### Results:

As shown in FIG. 11, compound 8 can depolarize Staphylococcus aureus cell membrane under sub-inhibitory concentration and hyperpolarize Staphylococcus aureus cell membrane under inhibitory concentration. Membrane potential was monitored by DiSc₃(5) over time (s).

### Example 13—Compounds 8 can Induce Calcium Influx into Cytoplasm

Briefly, an overnight culture of MRSA Mu3 was sub-cultured 1:100 in fresh CAMH and regrown to exponential phase. The bacteria were washed twice and then re-suspended in PBS (OD₅₉₅=0.5) containing 5 μM Fluo-4-AM. The bacteria were further incubated at 37° C. in dark for 2 hours before they were washed twice and resuspended into PBS containing 0.5 mM calcium chloride. 200 μL bacteria culture were then added into 96-well plate for each well. The plate was then pre-warmed and the fluorescence was monitored for 10 minutes for background measurement (excitation wavelength: 485 nm, emission wavelength: 535 nm). Fluorescence signal was recorded by Beckman Coulter DTX 880 Multimode Detector. Compounds were then added to 96-well plate and the fluorescence was further monitored for 5 minutes. Ionomycin, a calcium ionophore, was used as the positive control.

### Results:

As shown in FIG. 12, compound 8 can induce calcium influx into cytoplasm in concentration dependent manner. Cytoplasmic calcium ion content was monitored Fluo-4-am over time (s).

### Example 14—Addition of Calcium Channel Blockers can Antagonize the Antibacterial Effect of Compound 8 on Staphylococcus aureus

Exponential phase Staphylococcus aureus Mu3 was treated with 12.5 μM compound 8 (4×MIC) or combination with calcium channel blocker verapamil or benidipine. Data were representative of mean±S.D. of four individual experiments with error bars. Statistical analysis was performed by one-way ANOVA with Dunnett's post-test to calculate p-values adjusted for multiple comparisons with initial bacteria inoculum as control condition (*p<0.05; **p<0.01; ***p<0.001).

### Results:

As shown in FIG. 13, calcium channel blockers can antagonize the antibacterial effect of compound 8 on Staphylococcus aureus in dose dependent manner. This demonstrate calcium influx attributed to the bactericidal activity of compound 8.

### Example 15—Compound 8 and Compound 35 Accelerate Autolysis

The overnight culture of Staphylococcus aureus ATCC 29213 was subcultured, regrown to exponential phase and then diluted to OD₅₉₅=0.4. Bacteria were treated by 10×MIC of compound 8 and compound 35. At T=24 hour, OD₅₉₅ was measured.

### Results:

As shown in FIGS. 14A-14B, the addition of compound 8 and compound 35 lead to the reduction of the optical density of the Staphylococcus aureus culture within 24 hours, indicating accelerated autolysis.

### Example 16—Compound 8 has Diminished Bactericidal Activity in Autolysin Deletion Mutants of Staphylococcus aureus

Knockout of Autolysins Gene in Staphylococcus aureus

Target DNA upstream 1 kb and downstream 1 kb were PCR amplified from Staphylococcus aureus genomic DNA. The two DNA inserts were ligated to fuse upstream and downstream DNA fragments by overlap PCR. The ligated PCR product was cloned to pKOR1 plasmid using Vazyme ClonExpress®II One Step Cloning Kit, and transformed into Escherichia. coli top 10, plated on LBA containing ampicillin (100 μg/mL), and incubated under 30° C. overnight. Colony PCR was performed with Sanger sequencing for selection of transformants. The plasmid DNA was extracted and subsequently transformed into Staphylococcus aureus RN4220 and ATCC 29213 in TSA containing chloramphenicol (10 μg/mL). An isolated colony from ATCC 29213 transformant plates was picked and inoculated in TSB containing chloramphenicol (10 μg/mL) at 43° C. with vigorous shaking. The culture was streaked on TSA containing chloramphenicol (10 μg/mL) and incubated overnight at 43° C. A large colony was picked and inoculated in TSB containing chloramphenicol (10 μg/mL) at 30° C. overnight. The culture was diluted 10,000 times and 10 μL diluted culture was plated on TSA plates containing 100 ng/mL anhydrotetracycline.

The plate was incubated at 30° C. overnight. Twenty large colonies were picked and inoculated in 200 μL PBS. 10 μL bacteria were spotted on TSA containing chloramphenicol (10 μg/mL) and 10 μL bacteria were spotted on TSA without chloramphenicol. Bacteria isolates that could grow on TSA but not TSA containing chloramphenicol (10 μg/mL) was kept and cultured in TSB overnight at 37° C. The genome DNA was extracted to check for replacement of target gene using the primers 1 kb upstream and 1 kb downstream of the genes.

Bactericidal Activity of Compound 8 and Compound 35 in Autolysin Deficient Staphylococcus aureus

Staphylococcus aureus ATCC29213 WT, Staphylococcus aureus ATCC29213 Δatl, ΔSAOUHSC_02855 and ΔSAOUHSC_01895 was incubated with 4×MIC compound 8 (12.5 μM) for 24 hours. 10 μL bacteria were serial diluted and spotted on Mueller Hinton Agar. Colonies were enumerated next day. Data represented the mean±S.D. of biological triplicates. Statistical analysis was performed by unpaired t test compared to wild type in 95% confidence interval (*p<0.05; **p<0.01).

### Results:

As shown in FIG. 15, compound 8 has diminished bactericidal activity in autolysin deficient Staphylococcus aureus, which suggesting compound 8 kills bacteria via acceleration of the autolysis.

### Example 17—Compound 8 and Compound 35 Lead to Osmotic Burst of Staphylococcus aureus

### Field Emission Scanning Electron Microscopy

Briefly, an overnight culture of Staphylococcus aureus ATCC 29213 was sub-cultured 1:100 in fresh CAMH and regrown for 2 hours. Then the bacteria were treated with compound or DMSO negative control for 24 hours. The bacterial pellet was resuspended in PBS and loaded on 0.22 μm filter membrane. The bacteria were fixed on the membrane by 2.5% glutaraldehyde for overnight at 4° C. The filter membranes were washed by PBS and subsequently dehydrated using ethanol as shown below.

| | Volume (mL) | Number of times | Duration (min) |
|---|---|---|---|
| 30% ethanol | 5 | 2 | 15 |
| 50% ethanol | 5 | 2 | 15 |
| 70% ethanol | 5 | 2 | 15 |
| 90% ethanol | 5 | 2 | 20 |
| 100% ethanol | 5 | 3 | 20 |

The samples were submitted to HKU Electron Microscope Unit (EMU) for critical point drying, mounting and coating of the specimens. Samples were then observed using LEO 1530 FEG Scanning Electron Microscope.

### Transmission Electron Microscopy

Briefly, an overnight culture of Staphylococcus aureus ATCC 29213 was sub-cultured 1:100 in fresh CAMH and regrown for 2 hours. Then the bacteria were treated with compound or negative control DMSO for 24 hours. The bacterial pellet was then washed by PBS and fixed in cacodylate buffer (0.1 M sodium cacodylate-HCl buffer pH 7.4) for 1 hour at 4° C. The samples were then submitted to HKU EMU for osmium tetroxide (OsO₄) fixation, ultrathin sectioning and uranyl acetate staining. Samples were then observed using Philips CM100 Transmission Electron Microscope.

### Results:

As shown in FIGS. 16A-16B, both compound 8 and compound 35 treated Staphylococcus aureus showed severe cell abnormality including cell burst and irregular shape of cell wall. This suggested the acceleration of autolysis which weakened the cell wall, leading to the osmotic burst of the bacteria.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Tong, S. Y., Davis, J. S., Eichenberger, E., Holland, T. L., & Fowler, V. G. Jr. Staphylococcus aureus infections: epidemiology, pathophysiology, clinical manifestations, and management. Clin Microbiol Rev. 28, 603-61 (2015).
2. Lew, D. P. & Waldvogel, F. A. Osteonyelitis. Lancet. 364, 369-79 (2004).
3. Fisher, R., Gollan, B. & Helaine, S. Persistent bacterial infections and persister cells. Nat Rev Microbiol 15, 453-464 (2017).
4. Flemming, H. C., Wingender, J., Szewzyk, U. et al. Biofilms: an emergent form of bacterial life. Nat Rev Microbiol 14, 563-575 (2016).
5. Fux, C. A., Costerton J. W., Stewart, P. S. & Stoodley, P. Survival strategies of infectious biofilms. Trends Microbiol 13, 34-40 (2005).
6. Mitchell, P. Chemiosmotic coupling in oxidative and photosynthetic phosphorylation. Biochim Biophys Acta 1807, 1507-1538 (2011)
7. Kovacs, C. J., Faustoferri, R. C. & Quivey, R. G. Jr. RgpF Is Required for Maintenance of Stress Tolerance and Virulence in Streptococcus mutans. J Bacteriol 199, e00497-17 (2017)
8. Koul, A., Vranckx, L., Dendouga, N. et al. Diarylquinolines are bactericidal for dormant mycobacteria as a result of disturbed ATP homeostasis. J. Biol. Chem 283, 25273-25280 (2008)
9. Ikonomidis, A., Tsakris, A., Kanellopoulou, M., Maniatis, A. N. & Pournaras, S. Effect of the proton motive force inhibitor carbonyl cyanide-m-chlorophenylhydrazone (CCCP) on Pseudomonas aeruginosa biofilm development. Lett Appl Microbiol. 47, 298-302 (2008)
10. Wang, M., Chan, E. W. C., Wan, Y. et al. Active maintenance of proton motive force mediates starvation-induced bacterial antibiotic tolerance in Escherichia coli. Commun Biol 4, 1068 (2021).
11. Batzer, M. A. & Carlton, J. E., Deininger, P. L. Enhanced evolutionary PCR using oligonucleotides with inosine at the 3′-terminus. Nucleic Acid Res. 19, 5081 (1991)
12. Ohtsuka, E., Matsuki, S., Ikehara, M., Takahashi, Y. & Matsubara, K. An alternative approach to deoxyoligonucleotides as hybridization probes by insertion of deoxyinosine at ambiguous codon positions. J. Biol. Chem. 260, 2605-2608 (1985)
13. Rossolini, G. M., Cresti, S., Ingianni, A., Cattani, P., Riccio, M. L. & Satta, G. Use of deoxyinosine-containing primers vs degenerate primers for polymerase chain reaction based on ambiguous sequence information. Mol. Cell. Probes 8:91-98 (1994)
14. Shen, F.-F. in Design and synthesis of α-aminoxy acid-based cation transporters and their applications as anti-cancer and antibacterial agents, PhD, The University of Hong Kong, Hong Kong, (2018)
15. Yang, D., Li, B., Ng, F.-F., Yan, Y.-L., Qu, J., &Wu, Y.-D. Synthesis and Characterization of Chiral N—O Turns Induced by α-Aminoxy Acids J. Org. Chem., 66, 7303-7312 (2001)
16. Zha, H.-Y. in Design, synthesis and characterization of synthetic ion transporters, PhD, The University of Hong Kong, Hong Kong (2012)
17. Shin, I., Lee, M., Lee, J., Jung, M., Lee, W., Yoon, J. Synthesis of Optically Active Phthaloyl D-Aminooxy Acids from L-Amino Acids or L-Hydroxy Acids as Building Blocks for the Preparation of Aminooxy Peptides J. Org. Chem., 65, 7667-7675 (2000)
18. Gries, C. M., Sadykov, M. R., Bulock, L. L., Chaudhari, S. S., Thomas, V. C., Bose, J. L., Bayles, K. W. Potassium Uptake Modulates Staphylococcus aureus Metabolism. mSphere 1, e00125-16 (2016)

We claim:

1. A compound having Formula (LVIII):
A-L₁-(B)ₙ-L₂-D Formula (LVIII)
wherein A is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, substituted or unsubstituted alkyl, substituted or unsubstituted alkenyl, substituted or unsubstituted alkynyl, substituted or unsubstituted C₃-C₂₀ cycloalkyl, substituted or unsubstituted C₃-C₂₀ heterocyclyl, or an electron withdrawing group;
wherein each B independently has the structure: 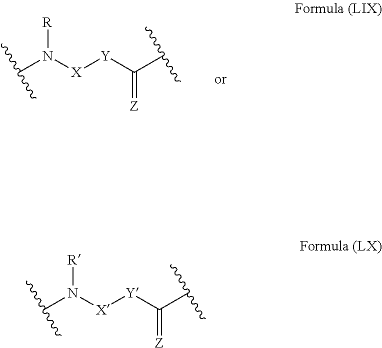
wherein X and X′ are independently O, absent, S, NR₂, substituted or unsubstituted alkyl, substituted or unsubstituted alkylene, or substituted or unsubstituted C₃-C₂₀ heterocycyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted aminoalkyl, substituted or unsubstituted aryl, substituted or unsubstituted alkaryl, substituted or unsubstituted aralkyl;
wherein Y and Y′ are independently substituted or unsubstituted alkyl, absent, O, S, NR₃, substituted or unsubstituted alkylene, substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted heteroaralkyl, substituted or unsubstituted polyaralkyl, substituted or unsubstituted polyheteroaralkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocycyl, or substituted or unsubstituted aminoalkyl;
wherein Z is O, S, or NR₄;
wherein L₁ and L₂ are independently —OC(O)—, —C(O)—, —S(O)₂—, —O, absent, —C(O)O—, —S(O)—, —C(O)NH—, —C(O)NR^{iv}—, —NR^{iv}C(O)—, —C(O)OCH₂—, —SO₂NR^{iv}—, —CH₂R^{iv}—, —NR^{iv}H—, —NR^{iv}—, —OCONH—, —NHCOO—, —OCONR^{iv}—, —NR^{iv}COO—, —NHCONH—, —NR^{iv}CONH—, —NHCONR^{iv}—, —NR^{iv}CONR^{iv}—, —CHOH—, —CR^{iv}OH—, substituted or unsubstituted alkyl, substituted or unsubstituted alkylene, substituted or unsubstituted alkenyl, substituted or unsubstituted alkylamino, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl;
wherein R₁, R₂, R₃, R₄, R₅, R, R′, R″, R′″, and R^{iv} are independently hydrogen, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂ heterocyclyl, substituted or unsubstituted aminoalkyl, substituted or unsubstituted aryl, substituted or unsubstituted alkaryl, substituted or unsubstituted aralkyl, or substituted or unsubstituted acyl;
wherein D is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, substituted or unsubstituted alkyl, substituted or unsubstituted alkenyl, substituted or unsubstituted alkynyl, substituted or unsubstituted C₃-C₂₀ cycloalkyl, substituted or unsubstituted C₃-C₂₀ cycloalkenyl, substituted or unsubstituted C₃-C₂₀ cycloalkynyl, unsubstituted or substituted C₃-C₂ heterocyclyl, substituted or unsubstituted aminoalkyl, substituted or unsubstituted alkaryl, substituted or unsubstituted carboxyalkyl, substituted or unsubstituted alkoxycarbonyl, substituted or unsubstituted acyl, or substituted or unsubstituted aminocarbonyl, or H; and wherein n is an integer from 1 to 5.

2. The compound of claim 1,
wherein A is substituted or unsubstituted aryl, substituted or unsubstituted heteroaryl, substituted or unsubstituted polyaryl, substituted or unsubstituted polyheteroaryl, substituted or unsubstituted aralkyl, substituted or unsubstituted heteroaralkyl, substituted or unsubstituted polyaralkyl, substituted or unsubstituted polyheteroaralkyl, or substituted or unsubstituted alkyl; and
L₁ is —OC(O)—, —C(O)—, or —S(O)₂—.

3. The compound of claim 2, wherein A is a substituted or unsubstituted aryl, substituted or unsubstituted aralkyl, or substituted or unsubstituted alkyl, having the formula (R₇-)ₐ(aryl), (R₇-)ₐ(aralkyl), or (R₇-)ₐ(alkyl), respectively, wherein each R₇ is independently F₃C—, F—, Cl—, O₂N—, NC—, MeO—, HO—, HC(O)—, (R₈)₂N—, wherein each R₈ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted aralkyl, or substituted or unsubstituted acyl, and wherein a is 0, 1, 2, or 3.

4. The compound of claim 1, wherein A is an electron withdrawing group.

5. The compound of claim 4, wherein the electron withdrawing group is CF₃, NO₂, F, Cl, Br, I, CN, CHO, or substituted or unsubstituted carbonyl, sulfonyl, trifluoroacetyl, or trifluoromethyl sulfonyl.

6. The compound of claim 1, wherein A is unsubstituted or substituted C₁₋₁₈ alkyl, substituted or unsubstituted aryl, or substituted or unsubstituted aralkyl, wherein the substituted C₁₋₁₈ alkyl, substituted aryl, or substituted aralkyl comprises an oxygen-, nitrogen-, or sulfur-containing moiety.

7. The compound of claim 1, wherein at least one of the atoms adjacent to B is oxygen, nitrogen, or sulfur.

8. The compound of claim 1, wherein A is substituted or unsubstituted heterocyclyl, wherein the substituted heterocyclyl is optionally substituted with an oxygen-, nitrogen-, or sulfur-containing moiety, or an electron withdrawing group.

9. The compound of claim 8, wherein the electron withdrawing group is CF₃, NO₂, F, Cl, Br, I, CN, or CHO.

10. The compound claim 1, wherein each Y or Y′ is independently substituted or unsubstituted C₁₋₁₀ alkyl, substituted or unsubstituted aralkyl, wherein the substituted alkyl or substituted aralkyl is O, S, NR₃, or an electron withdrawing group.

11. The compound of claim 10, wherein the electron withdrawing group is CF₃, NO₂, F, Cl, Br, I, CN, or CHO.

12. The compound of claim 1, wherein L₂ is NR₁₀, O, S, or absent, wherein R₁₀ is H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl;
wherein D is H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C3-C₂₀ cycloalkynyl, substituted or unsubstituted C₃-C₂₀ heterocyclyl, aminoalkyl, substituted or unsubstituted aryl, substituted or unsubstituted alkaryl, substituted or unsubstituted C₃-C₂₀ aralkyl, substituted or unsubstituted carboxyalkyl, substituted or unsubstituted C₃-C₂₀ alkoxycarbonyl, substituted or unsubstituted C₃-C₂₀ acyl, or substituted or unsubstituted C₃-C₂₀ aminocarbonyl.

13. The compound of claim 12, wherein D is a substituted or unsubstituted aryl having the formula -(aryl)(-R₁₁)_{b}, —R₁, or —O—R₁₁, wherein each R₁ is independently —CF₃, —F, C—Cl, —NO₂, —CN, —O-Me, —OH, —NR₁₂, wherein each R₁₂ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted C₃-C₂₀ cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl, wherein b is 0, 1, 2, or 3.

14. The compound of claim 1, wherein B is 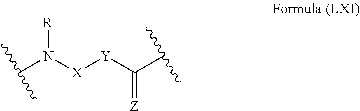
wherein X is O or absent, wherein Y is —CR₁₃R₁₄—, wherein R₁₃ is —CH₂-C₆H₅, —CH₂—O—C₆H₅, —C₁₋₄ alkyl, or —O—C₁₋₄ alkyl, wherein R₁₄ is H, wherein Z is O;
wherein n is 1;
wherein A has the formula (R₇-)ₐ(phenyl)-, L₁ is —C(O)—, wherein a is 2, wherein each R₇ is F₃C—;
wherein D is —CH₂—C₆H₅, —CH₂—O—C₆H₅, —C₁₋₄ alkyl, or —O—C₁₋₄ alkyl; and
wherein L₂ is NH, O, or S.

15. The compound of claim 1, wherein B is 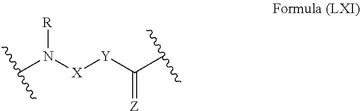
wherein X is O or absent,
wherein Y is —CR₁₃R₁₄—, wherein R₁₃ is —CH₂—C₆H₅, —CH₂—O—C₆H₅, —C₁₋₄ alkyl, —O—C₁₋₄ alkyl, —CH₂-(phenyl)(-R₁₅)ₑ, —CH—O-(phenyl)(-R₁₅)ₑ—C₁₋₄ alkyl-R₁₅, or —O—C₁₋₄ alkyl-R₁₅, wherein R₁₄ is H, wherein each R₁₅ is independently —CF₃, —F, C—Cl, —NO₂, —CN, —O-Me, —OH, —NH, wherein e is 1 or 2, wherein Z is O;
wherein n is 1;
wherein A is (R₇-)ₐ(phenyl)-CO—, C₆H₅—CH₂—, C₆H₅—O—CH₂—, C₁₋₄ alkyl-, or C₁₋₄ alkyl-O—, wherein a is 2, wherein each R₇ is independently F₃C—, F—, Cl—, O₂N—, NC—, MeO—, HO—, HN—;
L₁ is —C(O)— or absent;
wherein D is —CH₂—C₆H₅, —CH₂—O—C₆H₅, —C₁₋₄ alkyl, —O—C₁₋₄ alkyl, —CH₂-(phenyl)(-R₁₆)_{d}, —CH₂—O-(phenyl)(-R₁₆)_{d}, —C₁₋₄ alkyl-R₁₆, or —O—C₁₋₄ alkyl-R₁₆, wherein each R₁₆ is independently —CF₃, —F, C—Cl, —NO₂, —CN, —O-Me, —OH, —NH, wherein d is 1 or 2; and
wherein L₂ is NH, O, or S.

16. The compound of claim 1, wherein the compound is Formula (LXII), Formula (LXIII), or Formula (LXIV): 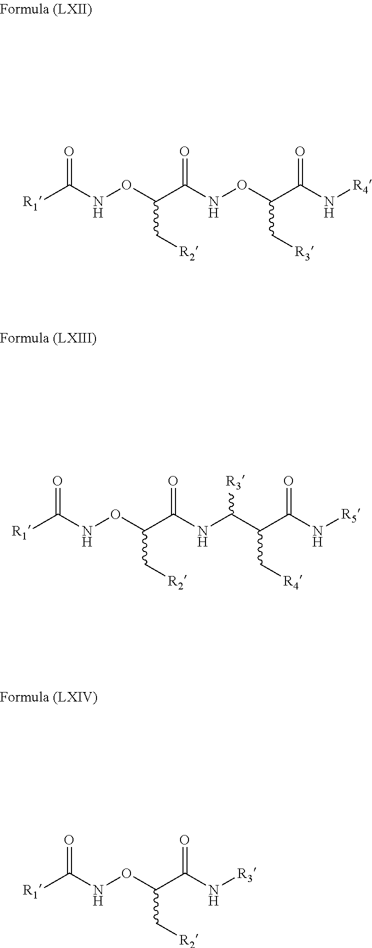
wherein each R₁′-R₅′ is independently H, substituted or unsubstituted alkyl, substituted or unsubstituted alkenyl, substituted or unsubstituted alkynyl, substituted or unsubstituted alkoxyalkyl, substituted or unsubstituted heteroalkyl, substituted or unsubstituted C₃-C₂₀ cycloalkyl, substituted or unsubstituted cycloalkenyl, substituted or unsubstituted C₃-C₂₀ cycloalkynyl, substituted or unsubstituted C₃-C₂₀ heterocyclyl, substituted or unsubstituted aminoalkyl, substituted or unsubstituted aryl, substituted or unsubstituted alkaryl, substituted or unsubstituted arylalkyl, substituted or unsubstituted carboxyalkyl, substituted or unsubstituted alkoxycarbonyl, substituted or unsubstituted acyl, or substituted or unsubstituted aminocarbonyl.

17. The compound of claim 1, wherein the compound is Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XVII), Formula (XVIII), Formula (XXIX), Formula (XXX), Formula (XXXI), Formula (XXXII), Formula (XXXIII), Formula (XXXIV), Formula (XXXV), Formula (XXXVI), Formula (XXXVII), Formula (XXXVIII), Formula (XXXIX), Formula (XL), Formula (XLI), Formula (XLII), Formula (XLIII), Formula (XLIV), Formula (XLV), Formula (XLVI), Formula (XLVII), Formula (XLVIII), Formula (XLIX), Formula (L), Formula (LI), Formula (LII), Formula (XLIII), Formula (LIV), Formula (LV), Formula (LVI), or Formula (LVII):

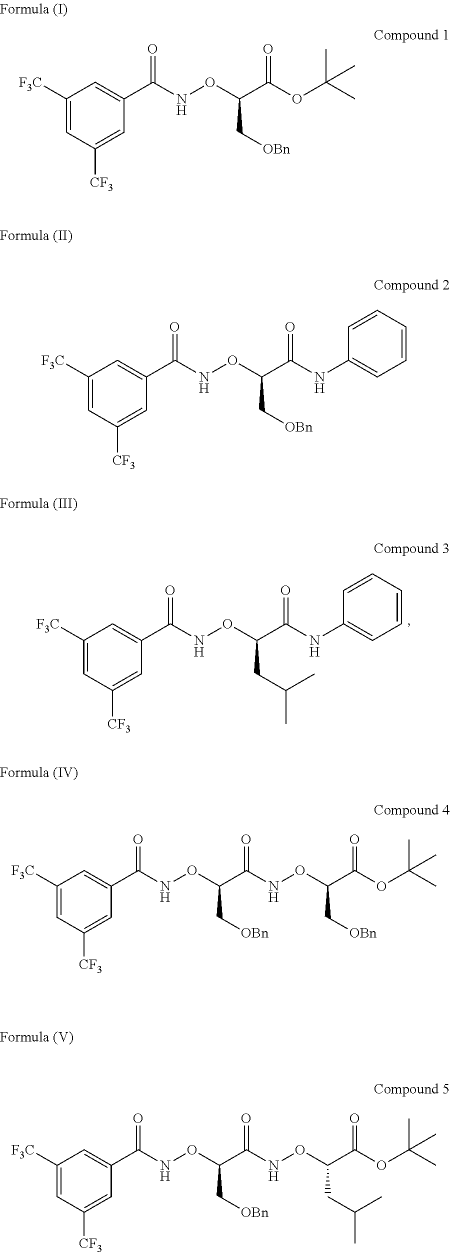 [SMILES: CC(C)(C)OC(=O)[C@@H](COCc1ccccc1)ONC(=O)c1cc(C(F)(F)F)cc(C(F)(F)F)c1 ; O=C(NO[C@H](COCc1ccccc1)C(=O)Nc1ccccc1)c1cc(C(F)(F)F)cc(C(F)(F)F)c1 ; CC(C)C[C@@H](ONC(=O)c1cc(C(F)(F)F)cc(C(F)(F)F)c1)C(=O)Nc1ccccc1 ; CC(C)(C)OC(=O)[C@@H](COCc1ccccc1)ONC(=O)[C@@H](COCc1ccccc1)ONC(=O)c1cc(C(F)(F)F)cc(C(F)(F)F)c1 ; CC(C)C[C@H](ONC(=O)[C@@H](COCc1ccccc1)ONC(=O)c1cc(C(F)(F)F)cc(C(F)(F)F)c1)C(=O)OC(C)(C)C]

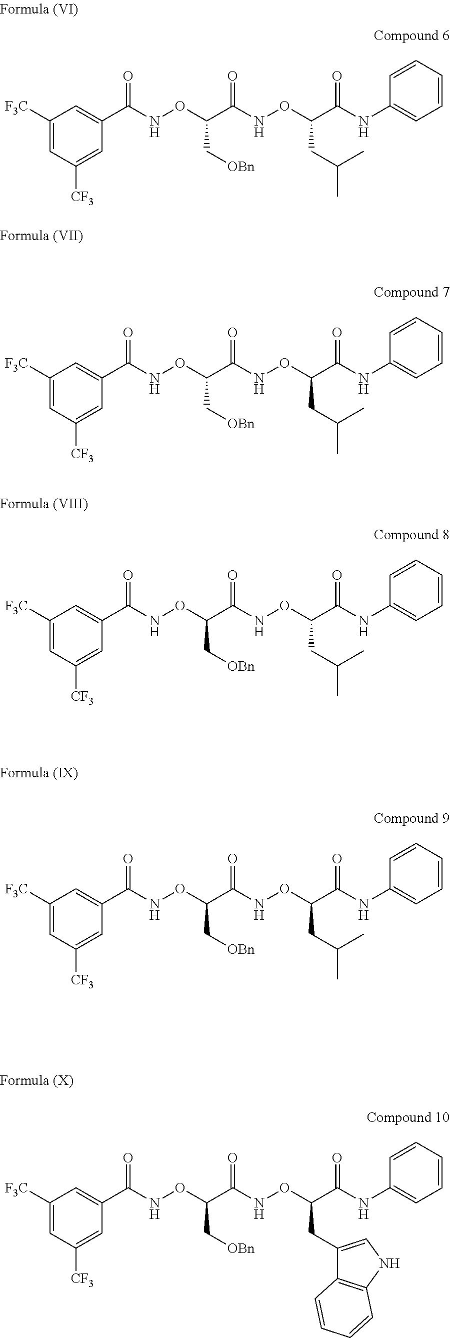 [SMILES: CC(C)C[C@H](ONC(=O)[C@H](COCc1ccccc1)ONC(=O)c1cc(C(F)(F)F)cc(C(F)(F)F)c1)C(=O)Nc1ccccc1 ; CC(C)C[C@@H](ONC(=O)[C@H](COCc1ccccc1)ONC(=O)c1cc(C(F)(F)F)cc(C(F)(F)F)c1)C(=O)Nc1ccccc1 ; CC(C)C[C@H](ONC(=O)[C@@H](COCc1ccccc1)ONC(=O)c1cc(C(F)(F)F)cc(C(F)(F)F)c1)C(=O)Nc1ccccc1 ; CC(C)C[C@@H](ONC(=O)[C@@H](COCc1ccccc1)ONC(=O)c1cc(C(F)(F)F)cc(C(F)(F)F)c1)C(=O)Nc1ccccc1 ; O=C(NO[C@H](COCc1ccccc1)C(=O)NO[C@H](Cc1c[nH]c2ccccc12)C(=O)Nc1ccccc1)c1cc(C(F)(F)F)cc(C(F)(F)F)c1]

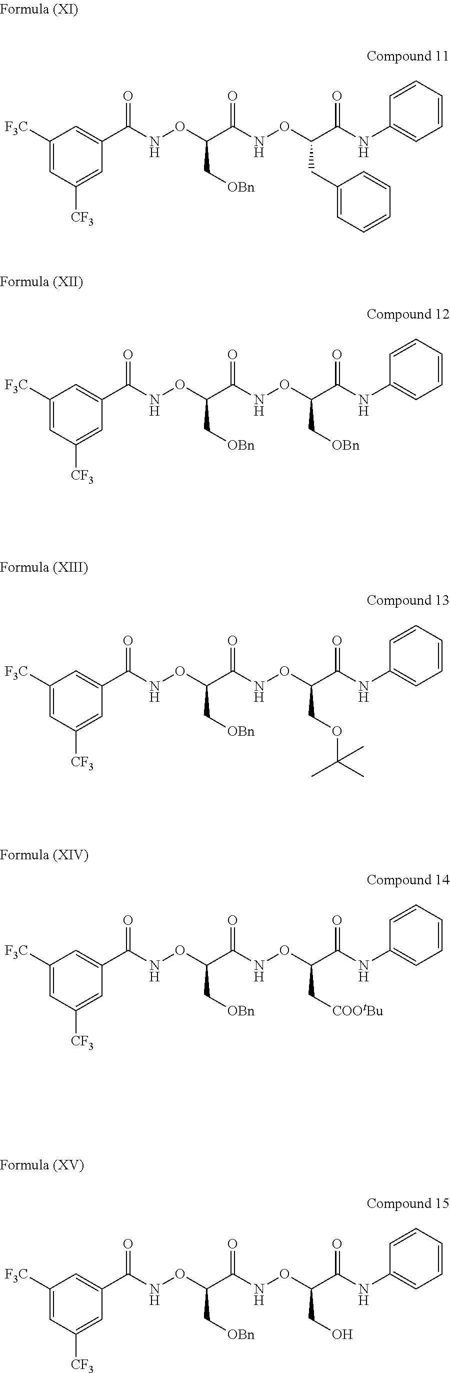

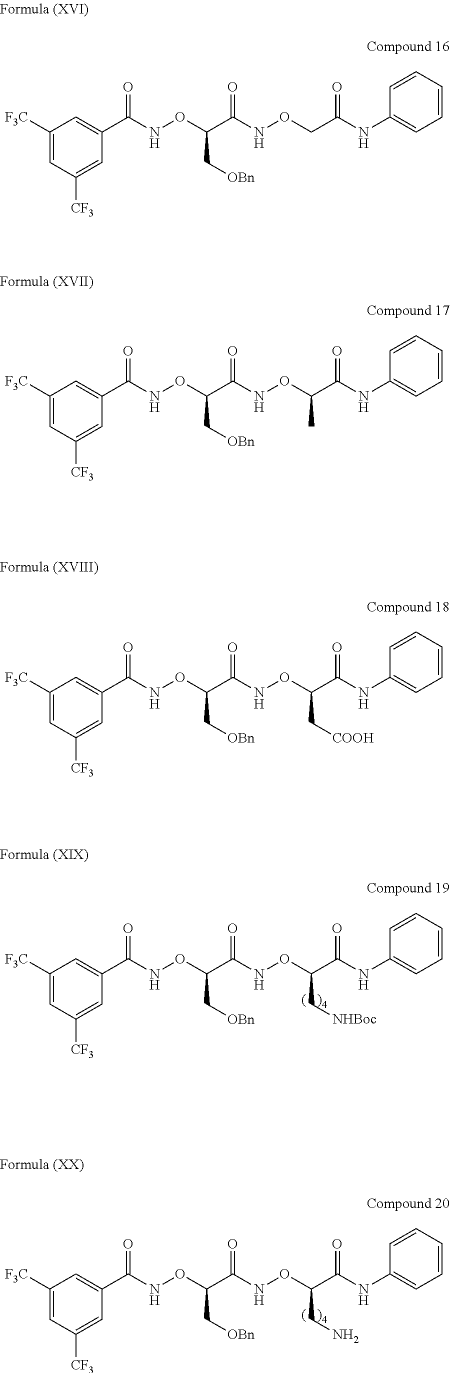

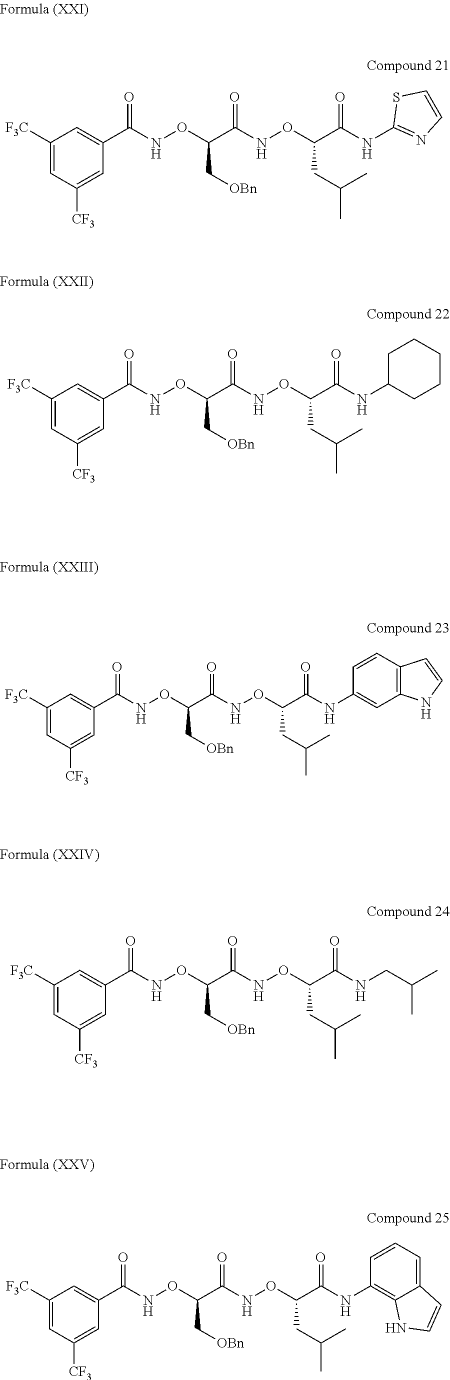 [SMILES: CC(C)C[C@H](ONC(=O)[C@@H](COCc1ccccc1)ONC(=O)c1cc(C(F)(F)F)cc(C(F)(F)F)c1)C(=O)Nc1nccs1 ; CC(C)C[C@H](ONC(=O)[C@@H](COCc1ccccc1)ONC(=O)c1cc(C(F)(F)F)cc(C(F)(F)F)c1)C(=O)NC1CCCCC1 ; CC(C)C[C@H](ONC(=O)[C@@H](COCc1ccccc1)ONC(=O)c1cc(C(F)(F)F)cc(C(F)(F)F)c1)C(=O)Nc1ccc2cc[nH]c2c1 ; CC(C)CNC(=O)[C@H](CC(C)C)ONC(=O)[C@@H](COCc1ccccc1)ONC(=O)c1cc(C(F)(F)F)cc(C(F)(F)F)c1 ; CC(C)C[C@H](ONC(=O)[C@@H](COCc1ccccc1)ONC(=O)c1cc(C(F)(F)F)cc(C(F)(F)F)c1)C(=O)Nc1cccc2cc[nH]c12]

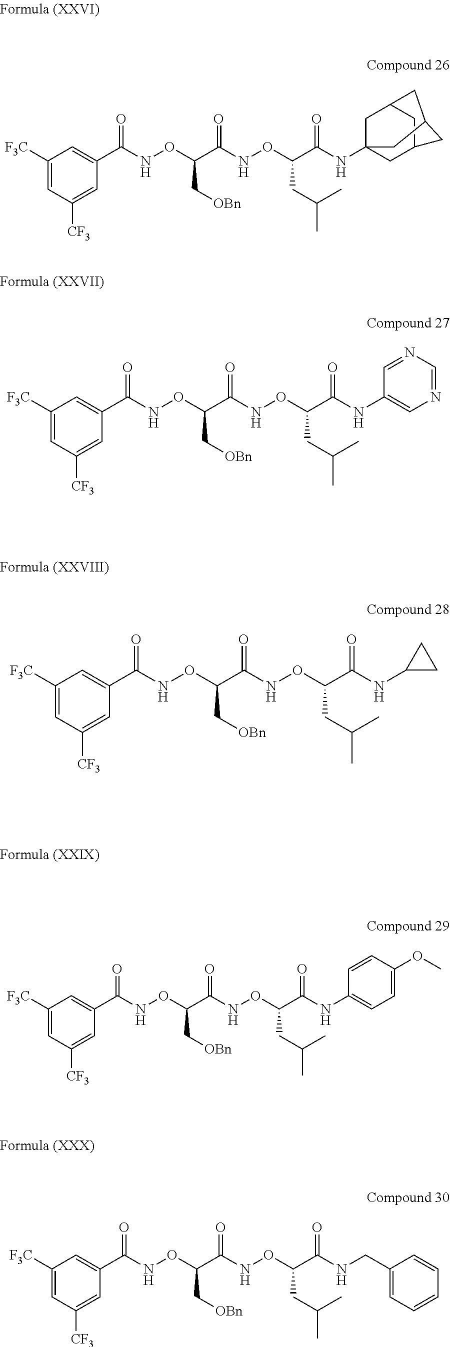 [SMILES: CC(C)C[C@H](ONC(=O)[C@@H](COCc1ccccc1)ONC(=O)c1cc(C(F)(F)F)cc(C(F)(F)F)c1)C(=O)NC12CC3CC(CC(C3)C1)C2 ; CC(C)C[C@H](ONC(=O)[C@@H](COCc1ccccc1)ONC(=O)c1cc(C(F)(F)F)cc(C(F)(F)F)c1)C(=O)Nc1cncnc1 ; CC(C)C[C@H](ONC(=O)[C@@H](COCc1ccccc1)ONC(=O)c1cc(C(F)(F)F)cc(C(F)(F)F)c1)C(=O)NC1CC1 ; COc1ccc(NC(=O)[C@H](CC(C)C)ONC(=O)[C@@H](COCc2ccccc2)ONC(=O)c2cc(C(F)(F)F)cc(C(F)(F)F)c2)cc1 ; CC(C)C[C@H](ONC(=O)[C@@H](COCc1ccccc1)ONC(=O)c1cc(C(F)(F)F)cc(C(F)(F)F)c1)C(=O)NCc1ccccc1]

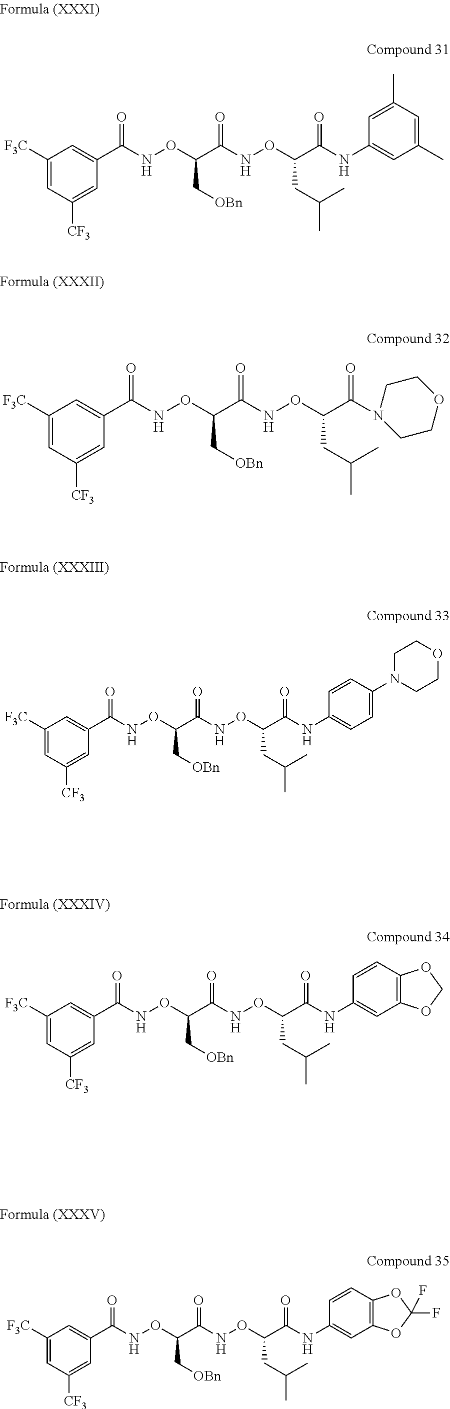 [SMILES: Cc1cc(C)cc(NC(=O)[C@H](CC(C)C)ONC(=O)[C@@H](COCc2ccccc2)ONC(=O)c2cc(C(F)(F)F)cc(C(F)(F)F)c2)c1 ; CC(C)C[C@H](ONC(=O)[C@@H](COCc1ccccc1)ONC(=O)c1cc(C(F)(F)F)cc(C(F)(F)F)c1)C(=O)N1CCOCC1 ; CC(C)C[C@H](ONC(=O)[C@@H](COCc1ccccc1)ONC(=O)c1cc(C(F)(F)F)cc(C(F)(F)F)c1)C(=O)Nc1ccc(N2CCOCC2)cc1 ; CC(C)C[C@H](ONC(=O)[C@@H](COCc1ccccc1)ONC(=O)c1cc(C(F)(F)F)cc(C(F)(F)F)c1)C(=O)Nc1ccc2c(c1)OCO2 ; CC(C)C[C@H](ONC(=O)[C@@H](COCc1ccccc1)ONC(=O)c1cc(C(F)(F)F)cc(C(F)(F)F)c1)C(=O)Nc1ccc2c(c1)OC(F)(F)O2]

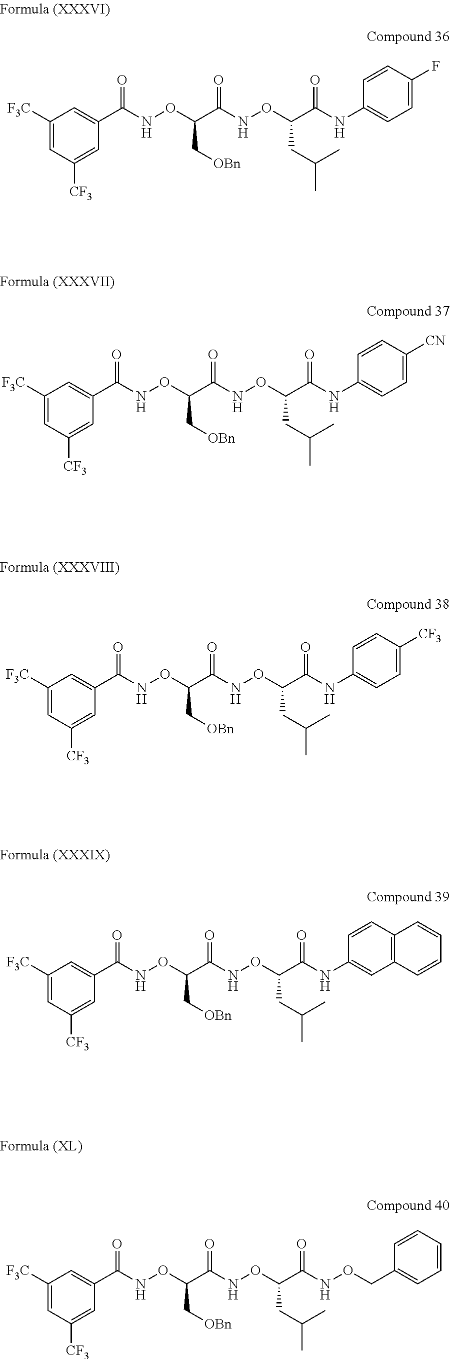 [SMILES: CC(C)C[C@H](ONC(=O)[C@@H](COCc1ccccc1)ONC(=O)c1cc(C(F)(F)F)cc(C(F)(F)F)c1)C(=O)Nc1ccc(F)cc1 ; CC(C)C[C@H](ONC(=O)[C@@H](COCc1ccccc1)ONC(=O)c1cc(C(F)(F)F)cc(C(F)(F)F)c1)C(=O)Nc1ccc(C#N)cc1 ; CC(C)C[C@H](ONC(=O)[C@@H](COCc1ccccc1)ONC(=O)c1cc(C(F)(F)F)cc(C(F)(F)F)c1)C(=O)Nc1ccc(C(F)(F)F)cc1 ; CC(C)C[C@H](ONC(=O)[C@@H](COCc1ccccc1)ONC(=O)c1cc(C(F)(F)F)cc(C(F)(F)F)c1)C(=O)Nc1ccc2ccccc2c1 ; CC(C)C[C@H](ONC(=O)[C@@H](COCc1ccccc1)ONC(=O)c1cc(C(F)(F)F)cc(C(F)(F)F)c1)C(=O)NOCc1ccccc1]

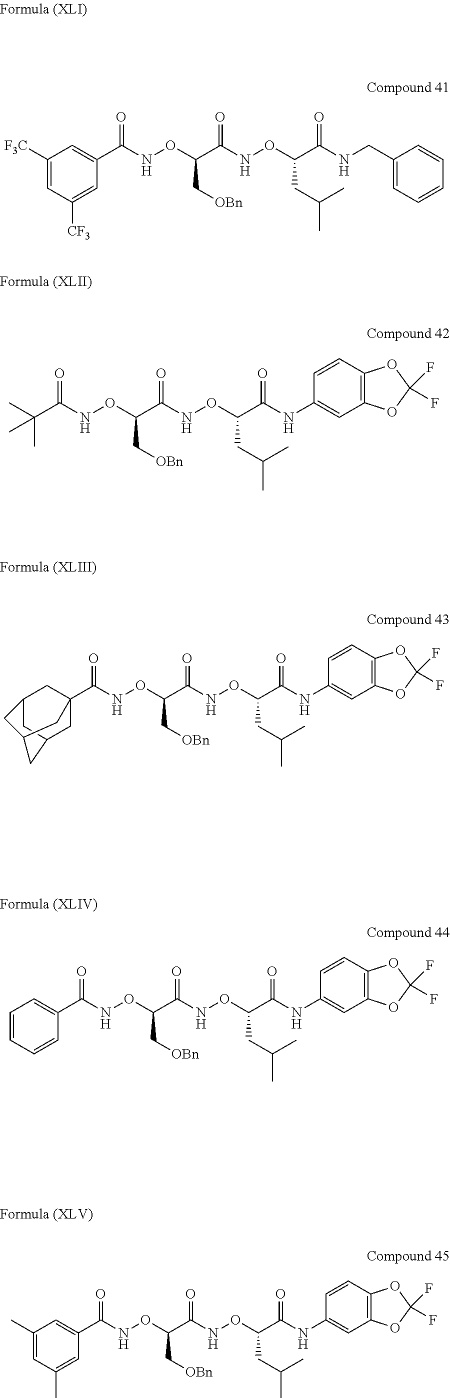 [SMILES: CC(C)C[C@H](ONC(=O)[C@@H](COCc1ccccc1)ONC(=O)c1cc(C(F)(F)F)cc(C(F)(F)F)c1)C(=O)NCc1ccccc1 ; CC(C)C[C@H](ONC(=O)[C@@H](COCc1ccccc1)ONC(=O)C(C)(C)C)C(=O)Nc1ccc2c(c1)OC(F)(F)O2 ; CC(C)C[C@H](ONC(=O)[C@@H](COCc1ccccc1)ONC(=O)C12CC3CC(CC(C3)C1)C2)C(=O)Nc1ccc2c(c1)OC(F)(F)O2 ; CC(C)C[C@H](ONC(=O)[C@@H](COCc1ccccc1)ONC(=O)c1ccccc1)C(=O)Nc1ccc2c(c1)OC(F)(F)O2 ; Cc1cc(C)cc(C(=O)NO[C@H](COCc2ccccc2)C(=O)NO[C@@H](CC(C)C)C(=O)Nc2ccc3c(c2)OC(F)(F)O3)c1]

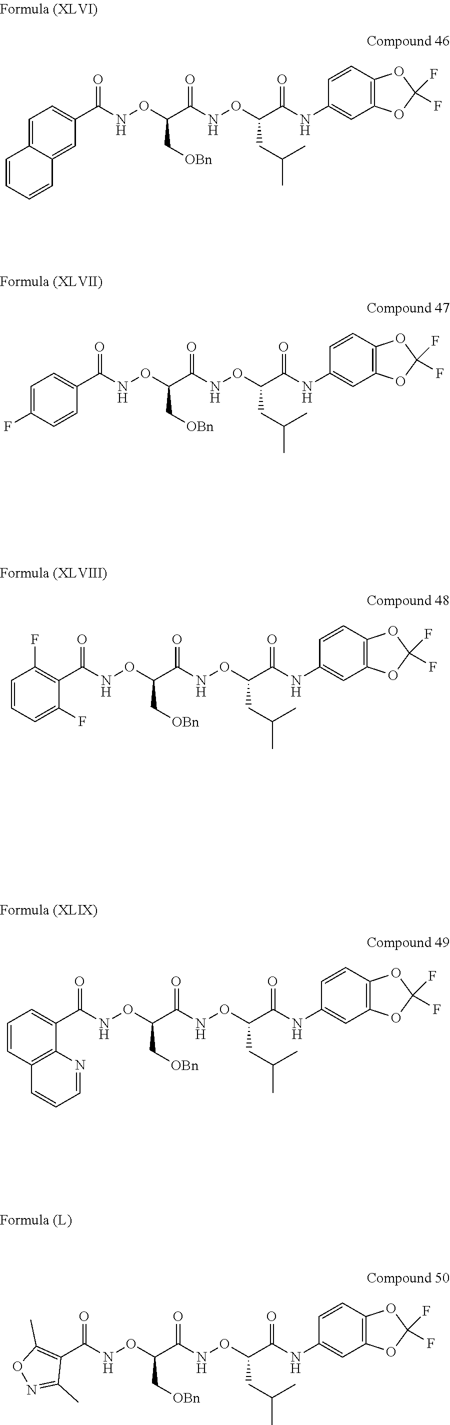 [SMILES: CC(C)C[C@H](ONC(=O)[C@@H](COCc1ccccc1)ONC(=O)c1ccc2ccccc2c1)C(=O)Nc1ccc2c(c1)OC(F)(F)O2 ; CC(C)C[C@H](ONC(=O)[C@@H](COCc1ccccc1)ONC(=O)c1ccc(F)cc1)C(=O)Nc1ccc2c(c1)OC(F)(F)O2 ; CC(C)C[C@H](ONC(=O)[C@@H](COCc1ccccc1)ONC(=O)c1c(F)cccc1F)C(=O)Nc1ccc2c(c1)OC(F)(F)O2 ; CC(C)C[C@H](ONC(=O)[C@@H](COCc1ccccc1)ONC(=O)c1cccc2cccnc12)C(=O)Nc1ccc2c(c1)OC(F)(F)O2 ; Cc1noc(C)c1C(=O)NO[C@H](COCc1ccccc1)C(=O)NO[C@@H](CC(C)C)C(=O)Nc1ccc2c(c1)OC(F)(F)O2]

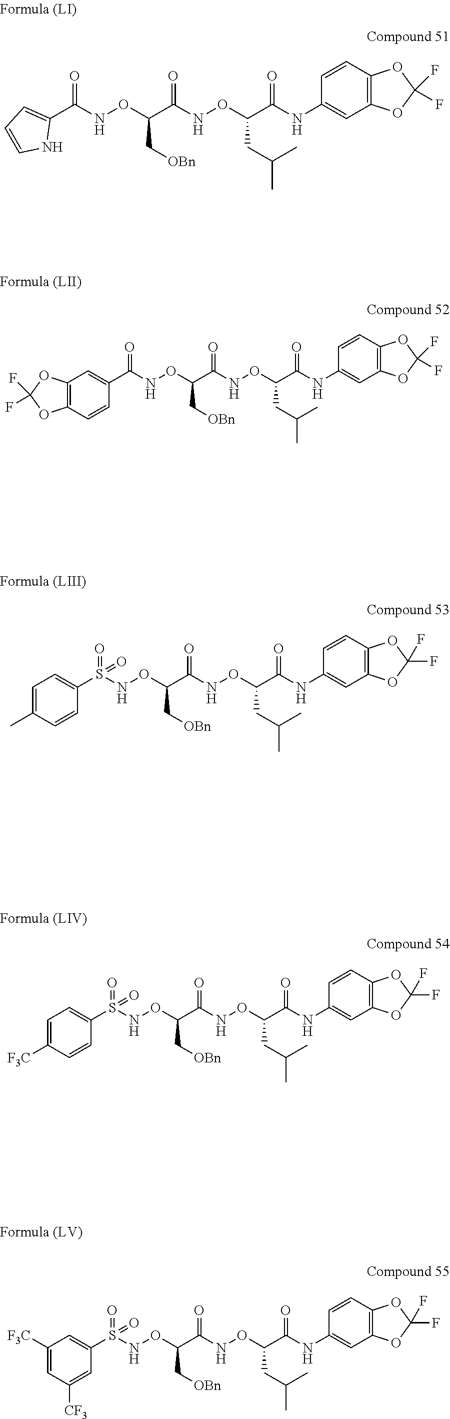 [SMILES: CC(C)C[C@H](ONC(=O)[C@@H](COCc1ccccc1)ONC(=O)c1ccc[nH]1)C(=O)Nc1ccc2c(c1)OC(F)(F)O2 ; CC(C)C[C@H](ONC(=O)[C@@H](COCc1ccccc1)ONC(=O)c1ccc2c(c1)OC(F)(F)O2)C(=O)Nc1ccc2c(c1)OC(F)(F)O2 ; Cc1ccc(S(=O)(=O)NO[C@H](COCc2ccccc2)C(=O)NO[C@@H](CC(C)C)C(=O)Nc2ccc3c(c2)OC(F)(F)O3)cc1 ; CC(C)C[C@H](ONC(=O)[C@@H](COCc1ccccc1)ONS(=O)(=O)c1ccc(C(F)(F)F)cc1)C(=O)Nc1ccc2c(c1)OC(F)(F)O2 ; CC(C)C[C@H](ONC(=O)[C@@H](COCc1ccccc1)ONS(=O)(=O)c1cc(C(F)(F)F)cc(C(F)(F)F)c1)C(=O)Nc1ccc2c(c1)OC(F)(F)O2]

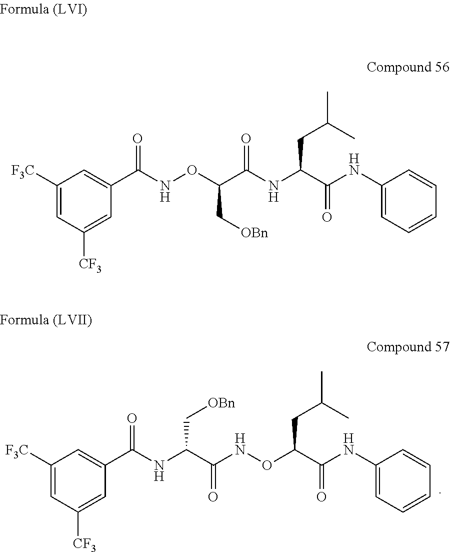 [SMILES: CC(C)C[C@H](NC(=O)[C@@H](COCc1ccccc1)ONC(=O)c1cc(C(F)(F)F)cc(C(F)(F)F)c1)C(=O)Nc1ccccc1 ; CC(C)C[C@H](ONC(=O)[C@@H](COCc1ccccc1)NC(=O)c1cc(C(F)(F)F)cc(C(F)(F)F)c1)C(=O)Nc1ccccc1]

18. A composition comprising the compound of claim 1 or a pharmaceutically acceptable salt, solvate, or hydrate thereof and at least one pharmaceutically acceptable carrier and/or excipient.

19. The composition of claim 18, further comprising an antibiotic.

20. The composition of claim 19, wherein the antibiotic is gentamicin.

21. A method of treating or inhibiting an infection by a gram-positive bacterium in a subject, the method comprising administering the compound of claim 1 and, optionally, at least one antibiotic, to the subject.

22. The method of claim 21, where the gram-positive bacterium is Staphylococcus aureus, Bacillus subtilis, Streptococcus pneumoniae, Listeria monocytogenes, Enterococcus faecalis, or Mycobacterium smegmatis.

23. The method of claim 21, wherein the gram-positive bacterium is a persister cell or is in a biofilm.

24. The method of claim 21, wherein the infection by the gram-positive bacterium is treated or inhibited by altering the proton motive force of the gram-positive bacterium.

25. The method of claim 24, where the altering of the proton motive force comprises membrane potential hyperpolarization, calcium ion influx, accelerated autolysis, or any combination thereof.

26. The method of claim 21, wherein the at least one antibiotic is gentamicin.


SEQUENCE LISTING (7 sequences)

SEQ ID NO: 1 - DNA, 15 bp, synthetic construct
  source 1..15: mol_type = other DNA
ctgcaggcatgcaag

SEQ ID NO: 2 - DNA, 23 bp, synthetic construct
  source 1..23: mol_type = other DNA
gtttaaaacctccctagtcgacg

SEQ ID NO: 3 - DNA, 45 bp, synthetic construct
  source 1..45: mol_type = other DNA
atgtcaaaaggtgaagaattatttacaggtgttgttcctatttta

SEQ ID NO: 4 - DNA, 39 bp, synthetic construct
  source 1..39: mol_type = other DNA
ttatttatataattcatccataccatgtgtaatacctgc

SEQ ID NO: 5 - DNA, 55 bp, synthetic construct
  source 1..55: mol_type = other DNA
actagggaggttttaaacatgtcaaaaggtgaagaattatttacaggtgttgttc

SEQ ID NO: 6 - DNA, 57 bp, synthetic construct
  source 1..57: mol_type = other DNA
cttgcatgcctgcagttatttatataattcatccataccatgtgtaatacctgctgc

SEQ ID NO: 7 - DNA, 17 bp, synthetic construct
  source 1..17: mol_type = other DNA
gaaagggggatgtgctg